(19) United States
(12) Patent Application Publication
(10) Pub. No.: US 2020/0405401 A1
(43) Pub. Date: Dec. 31, 2020

(54) VISUALIZATION SYSTEM WITH AUTOMATIC CONTAMINATION DETECTION AND CLEANING CONTROLS

(71) Applicant: Ethicon LLC, Guaynabo, PR (US)

(72) Inventors: Frederick E. Shelton, IV, Hillsboro, OH (US); Jason L. Harris, Lebanon, OH (US); Daniel J. Mumaw, Liberty Township, OH (US); Kevin M. Fiebig, Cincinnati, OH (US)

(21) Appl. No.: 16/454,727

(22) Filed: Jun. 27, 2019

(51) Int. Cl. A61B 34/20; A61B 90/00; A61B 34/35

(52) U.S. Cl. CPC A61B 34/20; A61B 90/361; A61B 34/74; A61B 34/35; A61B 2090/371; A61B 90/37

(57) ABSTRACT

A surgical visualization system is disclosed including an imaging device including an imaging lens, a lens cleaning system, a user interface, and a control circuit. The control circuit is configured to monitor a parameter indicative of lens transparency of the imaging lens and present, through the user interface, a lens transparency level based on the parameter.

### BACKGROUND

The present disclosure relates to robotic surgical systems. Robotic surgical systems can include a central control unit, a surgeon's command console, and a robot having one or more robotic arms. Robotic surgical tools can be releasably mounted to the robotic arm(s). The number and type of robotic surgical tools can depend on the type of surgical procedure. Robotic surgical systems can be used in connection with one or more displays and/or one or more handheld surgical instruments during a surgical procedure.

### FIGURES

The features of various aspects are set forth with particularity in the appended claims. The various aspects, however, both as to organization and methods of operation, together with further objects and advantages thereof, may best be understood by reference to the following description, taken in conjunction with the accompanying drawings as follows.

FIG. 1 is a block diagram of a computer-implemented interactive surgical system, in accordance with at least one aspect of the present disclosure.

FIG. 2 is a surgical system being used to perform a surgical procedure in an operating room, in accordance with at least one aspect of the present disclosure.

FIG. 3 is a surgical hub paired with a visualization system, a robotic system, and an intelligent instrument, in accordance with at least one aspect of the present disclosure.

FIG. 4 is a schematic of a robotic surgical system, in accordance with at least one aspect of the present disclosure.

FIG. 4A illustrates another exemplification of a robotic arm and another exemplification of a tool assembly releasably coupled to the robotic arm, according to one aspect of the present disclosure.

FIG. 5 is a block diagram of control components for the robotic surgical system of FIG. 4, in accordance with at least one aspect of the present disclosure.

FIG. 6 is a schematic of a robotic surgical system during a surgical procedure including a plurality of hubs and interactive secondary displays, in accordance with at least one aspect of the present disclosure.

FIG. 7 is a detail view of the interactive secondary displays of FIG. 6, in accordance with at least one aspect of the present disclosure.

FIG. 8 illustrates a surgical data network comprising a modular communication hub configured to connect modular devices located in one or more operating theaters of a healthcare facility, or any room in a healthcare facility specially equipped for surgical operations, to the cloud, in accordance with at least one aspect of the present disclosure.

FIG. 9 illustrates a computer-implemented interactive surgical system, in accordance with at least one aspect of the present disclosure.

FIG. 10 illustrates a surgical hub comprising a plurality of modules coupled to the modular control tower, in accordance with at least one aspect of the present disclosure.

FIG. 11 illustrates one aspect of a Universal Serial Bus (USB) network hub device, in accordance with at least one aspect of the present disclosure.

FIG. 12 illustrates a logic diagram of a control system of a surgical instrument or tool, in accordance with at least one aspect of the present disclosure.

FIG. 13 illustrates a control circuit configured to control aspects of the surgical instrument or tool, in accordance with at least one aspect of the present disclosure.

FIG. 14 illustrates a combinational logic circuit configured to control aspects of the surgical instrument or tool, in accordance with at least one aspect of the present disclosure.

FIG. 15 illustrates a sequential logic circuit configured to control aspects of the surgical instrument or tool, in accordance with at least one aspect of the present disclosure.

FIG. 16 illustrates a surgical instrument or tool comprising a plurality of motors which can be activated to perform various functions, in accordance with at least one aspect of the present disclosure.

FIG. 17 is a schematic diagram of a robotic surgical instrument configured to operate a surgical tool described herein, in accordance with at least one aspect of the present disclosure.

FIG. 18 illustrates a block diagram of a surgical instrument programmed to control the distal translation of a displacement member, in accordance with at least one aspect of the present disclosure.

FIG. 19 is a schematic diagram of a surgical instrument configured to control various functions, in accordance with at least one aspect of the present disclosure.

FIG. 20 is a simplified block diagram of a generator configured to provide inductorless tuning, among other benefits, in accordance with at least one aspect of the present disclosure.

FIG. 21 illustrates an example of a generator, which is one form of the generator of FIG. 20, in accordance with at least one aspect of the present disclosure.

FIG. 22 is a schematic of a robotic surgical system, in accordance with one aspect of the present disclosure.

FIG. 23 illustrates a surgical visualization system including a robotic arm coupled to a visualization assembly, in accordance with at least one aspect of the present disclosure.

FIG. 24 illustrates a perspective view of a distal portion of the visualization assembly of FIG. 23.

FIG. 25 illustrates a longitudinal cross-sectional view of the distal portion of the visualization assembly of FIG. 24.

FIG. 26 is a logic flow diagram of a process depicting a control program or a logic configuration for detecting lens transparency of a surgical visualization system and reporting the same, in accordance with at least one aspect of the present disclosure.

FIG. 26A is a is a logic flow diagram of a process depicting a control program or a logic configuration for determining whether a visualization lens of a surgical visualization system needs cleaning and triggering the cleaning, in accordance with at least one aspect of the present disclosure.

FIG. 27 is a schematic diagram of a surgical visualization system, in accordance with at least one aspect of the present disclosure.

FIG. 28 illustrates a perspective view of a distal portion of a visualization assembly of a surgical visualization system, in accordance with at least one aspect of the present disclosure.

FIG. 29 is a graph depicting time (t) on the x-axis and occlusion level through a visualization lens of a surgical visualization system on the y-axis, in accordance with at least one aspect of the present disclosure.

FIG. 30 illustrates two trocars inserted into a body cavity, the first trocar accommodating a visualization assembly, and the second trocar accommodating an electrosurgical instrument, in accordance with at least one aspect of the present disclosure.

FIG. 31 is a graph including a top graph that represents temperature on the Y-axis vs time on the X-axis and a bottom graph that represents lens visibility percentage on the Y-axis vs time on the X-axis, in accordance with at least one aspect of the present disclosure.

FIG. 32 is a graph including a top graph that represents temperature on the Y-axis vs time on the X-axis and a bottom graph that represents lens visibility percentage on the Y-axis vs time on the X-axis, in accordance with at least one aspect of the present disclosure.

FIG. 33 illustrates an imaging device including a distal end a distance D1 from an iris seal of a seal assembly of a trocar, in accordance with at least one aspect of the present disclosure.

FIG. 34 illustrates the imaging device inserted into the iris seal of the seal assembly of the trocar of FIG. 33, in accordance with at least one aspect of the present disclosure.

FIG. 35 illustrates a trocar connected to a lens cleaning system, in accordance with at least one aspect of the present disclosure.

FIG. 36 illustrates the trocar of FIG. 35 with an imaging device being cleaned inside the trocar by a flushing fluid from the lens cleaning system, in accordance with at least one aspect of the present disclosure.

FIG. 37 is an exploded view of a device.

FIG. 38 is an alternative embodiment of a portion of the device shown in FIG. 37.

FIG. 39 is a cross-sectional view of a mounting structure and cannula assembly.

FIG. 40 is a partial cross-sectional view showing a seal body housing.

FIG. 41 is a perspective view with parts separated of a cannula assembly.

FIG. 42 is an enlarged view of the indicated area of detail of FIG. 41.

FIG. 43 is a cross sectional view of a sealing cannula.

FIG. 44 is a perspective view of a pendent valve mounted to an end cap of a trocar.

FIG. 45 is an axial cross-section view illustrating operation of the pendent valve during off-axis insertion of an instrument.

FIG. 46 is an axial cross-section view showing an instrument fully inserted with effective seal formation notwithstanding an off-axis position of the instrument.

FIG. 47 is a cross-section view taken along lines 47-47 of FIG. 46.

FIG. 48 is a cross-section view taken along lines 48-48 of FIG. 46.

FIG. 49 is a perspective view of an assembled trocar.

FIG. 50 is an exploded perspective view of the components of the trocar of FIG. 49.

FIG. 51 is an exploded perspective view of a trocar assembly;

FIG. 52 is an exploded cross-sectional side view of an adaptor attached to a seal assembly positioned above a cannula.

FIG. 53 is a side cross-sectional view of a trocar assembly;

FIG. 54 is a cross-sectional side view of the trocar assembly of FIG. 53, in a first shifted condition.

FIG. 55 is an exploded view of an insertable seal system and a cross sectional view of a trocar assembly including the insertable seal system positioned therein.

FIG. 56 is an exploded view of a trocar assembly including a third seal with an insertable seal system.

FIG. 57 is a seal assembly positioned above a trocar assembly that is held by a robot arm of a robotic surgical system

FIG. 58 is a cross sectional view of a trocar assembly with a flexible seal housing.

FIG. 59 illustrates a surgical access device positioned in an intercostal space of a patient, in accordance with at least one aspect of the present disclosure.

FIG. 60 illustrates two ribs spread apart via a surgical retractor, and a surgical access device position between the ribs, in accordance with at least one aspect of the present disclosure.

FIG. 61 illustrates a surgical access device with three access ports facilitating access of three surgical tools into a patient thoracic cavity, wherein the surgical tools are controlled by three robotic arms, in accordance with at least one aspect of the present disclosure.

FIG. 62 illustrates a partial perspective view of the robotic arms and surgical tools of FIG. 61.

FIG. 63 illustrates a surgical access device with a single access port facilitating access of three surgical tools into a patient cavity, in accordance with at least one aspect of the present disclosure.

FIG. 64 is a surgical access device with a translatable member in a first position, in accordance with at least one aspect of the present disclosure.

FIG. 65 illustrates a surgical access device with a translatable member in a second position, in accordance with at least one aspect of the present disclosure.

FIG. 66 illustrates a translatable member of a surgical access device, in accordance with at least one aspect of the present disclosure.

FIG. 67 is a block diagram illustrating a control circuit for moving a translatable member of a surgical access device, in accordance with at least one aspect of the present disclosure.

FIG. 68 illustrates a partial perspective view of a robotic arm before assembly with a surgical access device and a surgical instrument, in accordance with at least one aspect of the present disclosure.

FIG. 69 illustrates a partial cross-sectional view of the robotic arm of FIG. 68 assembled with a surgical instrument and a surgical access device, in accordance with at least one aspect of the present disclosure.

FIG. 70 illustrates a partial cross-sectional view of the robotic arm of FIG. 68 assembled with a surgical instrument and a surgical access device, in accordance with at least one aspect of the present disclosure.

FIG. 71 illustrates a partial cross-sectional view of a surgical access device including stabilizing compartments, in accordance with at least one aspect of the present disclosure.

FIG. 72 illustrates a partial elevational view of a surgical instrument including dampening features, in accordance with at least one aspect of the present disclosure.

FIG. 73 illustrates the surgical instrument of FIG. 72 assembled with the surgical access device of FIG. 71, in accordance with at least one aspect of the present disclosure.

FIG. 74 illustrates a surgical access device with non-concentric instrument support features, in accordance with at least one aspect of the present disclosure.

FIG. 75 illustrates three transverse cross-sectional views of the surgical access device of FIG. 74, in accordance with at least one aspect of the present disclosure.

FIG. 76 is a schematic diagram illustrating a top view of the surgical access device of FIG. 74, in accordance with at least one aspect of the present disclosure.

FIG. 77 is a cross-sectional view of a port assembly shown with a surgical instrument extending through the interior space of the port assembly at an angle.

FIG. 78 is a side cross-sectional view of an access apparatus.

FIG. 79 is a side plan view of the seal assembly of the access apparatus of FIG. 78.

FIG. 80 is an enlarged isolated view in cross-section of FIG. 79, detailing the components of the seal of the access apparatus.

FIG. 81 is a side cross-sectional view of the access apparatus.

FIG. 82 is a view similar to the view of FIG. 81 illustrating insertion and manipulation of a surgical instrument within the access apparatus with the instrument rotating about a central axis of rotation defined by the access apparatus.

FIG. 83 is a side view of an example radial biasing device that may be used with a trocar assembly.

FIGS. 84 and 85 are cross-sectional side views of the radial biasing device of FIG. 83 depicting example operation.

FIG. 86 is a perspective view illustrating the obturator assembly mounted to the cannula assembly to permit the penetration of tissue.

### DESCRIPTION

Applicant of the present application owns the following U.S. Patent Applications, filed on even date herewith, the disclosure of each of which is herein incorporated by reference in its entirety:
Attorney Docket No. END9105USNP1/190145-1M, titled METHOD OF USING A SURGICAL MODULAR ROBOTIC ASSEMBLY;
Attorney Docket No. END9106USNP1/190146-1, titled SURGICAL SYSTEMS WITH INTERCHANGEABLE MOTOR PACKS;
Attorney Docket No. END9107USNP1/190147-1, titled COOPERATIVE ROBOTIC SURGICAL SYSTEMS;
Attorney Docket No. END9108USNP1/190148-1, titled HEAT EXCHANGE SYSTEMS FOR ROBOTIC SURGICAL SYSTEMS;
Attorney Docket No. END9108USNP2/190148-2, titled DETERMINING ROBOTIC SURGICAL ASSEMBLY COUPLING STATUS;
Attorney Docket No. END9108USNP3/190148-3, titled ROBOTIC SURGICAL ASSEMBLY COUPLING SAFETY MECHANISMS;
Attorney Docket No. END9109USNP1/190149-1, titled ROBOTIC SURGICAL SYSTEM WITH SAFETY AND COOPERATIVE SENSING CONTROL;
Attorney Docket No. END9109USNP2/190149-2, titled ROBOTIC SURGICAL SYSTEM FOR CONTROLLING CLOSE OPERATION OF END-EFFECTORS;
Attorney Docket No. END9109USNP3/190149-3, titled ROBOTIC SURGICAL SYSTEM WITH LOCAL SENSING OF FUNCTIONAL PARAMETERS BASED ON MEASUREMENTS OF MULTIPLE PHYSICAL INPUTS;
Attorney Docket No. END9110USNP1/190150-1, titled COOPERATIVE OPERATION OF ROBOTIC ARMS;
Attorney Docket No. END9111USNP1/190151-1, titled SURGICAL INSTRUMENT DRIVE SYSTEMS;
Attorney Docket No. END9111USNP2/190151-2, titled SURGICAL INSTRUMENT DRIVE SYSTEMS WITH CABLE-TIGHTENING SYSTEM; and
Attorney Docket No. END9112USNP2/190158-2, titled MULTI-ACCESS PORT FOR SURGICAL ROBOTIC SYSTEMS.

Applicant of the present application owns the following U.S. Patent Applications, filed on Dec. 4, 2018, the disclosure of each of which is herein incorporated by reference in its entirety:
U.S. patent application Ser. No. 16/209,385, titled METHOD OF HUB COMMUNICATION, PROCESSING, STORAGE AND DISPLAY;
U.S. patent application Ser. No. 16/209,395, titled METHOD OF HUB COMMUNICATION;
U.S. patent application Ser. No. 16/209,403, titled METHOD OF CLOUD BASED DATA ANALYTICS FOR USE WITH THE HUB;
U.S. patent application Ser. No. 16/209,407, titled METHOD OF ROBOTIC HUB COMMUNICATION, DETECTION, AND CONTROL;
U.S. patent application Ser. No. 16/209,416, titled METHOD OF HUB COMMUNICATION, PROCESSING, DISPLAY, AND CLOUD ANALYTICS;
U.S. patent application Ser. No. 16/209,423, titled METHOD OF COMPRESSING TISSUE WITHIN A STAPLING DEVICE AND SIMULTANEOUSLY DISPLAYING THE LOCATION OF THE TISSUE WITHIN THE JAWS;
U.S. patent application Ser. No. 16/209,427, titled METHOD OF USING REINFORCED FLEXIBLE CIRCUITS WITH MULTIPLE SENSORS TO OPTIMIZE PERFORMANCE OF RADIO FREQUENCY DEVICES;
U.S. patent application Ser. No. 16/209,433, titled METHOD OF SENSING PARTICULATE FROM SMOKE EVACUATED FROM A PATIENT, ADJUSTING THE PUMP SPEED BASED ON THE SENSED INFORMATION, AND COMMUNICATING THE FUNCTIONAL PARAMETERS OF THE SYSTEM TO THE HUB;
U.S. patent application Ser. No. 16/209,447, titled METHOD FOR SMOKE EVACUATION FOR SURGICAL HUB;
U.S. patent application Ser. No. 16/209,453, titled METHOD FOR CONTROLLING SMART ENERGY DEVICES;
U.S. patent application Ser. No. 16/209,458, titled METHOD FOR SMART ENERGY DEVICE INFRASTRUCTURE;
U.S. patent application Ser. No. 16/209,465, titled METHOD FOR ADAPTIVE CONTROL SCHEMES FOR SURGICAL NETWORK CONTROL AND INTERACTION;
U.S. patent application Ser. No. 16/209,478, titled METHOD FOR SITUATIONAL AWARENESS FOR SURGICAL NETWORK OR SURGICAL NETWORK CONNECTED DEVICE CAPABLE OF ADJUSTING FUNCTION BASED ON A SENSED SITUATION OR USAGE;
U.S. patent application Ser. No. 16/209,490, titled METHOD FOR FACILITY DATA COLLECTION AND INTERPRETATION; and
U.S. patent application Ser. No. 16/209,491, titled METHOD FOR CIRCULAR STAPLER CONTROL ALGORITHM ADJUSTMENT BASED ON SITUATIONAL AWARENESS.

Before explaining various aspects of surgical devices and generators in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

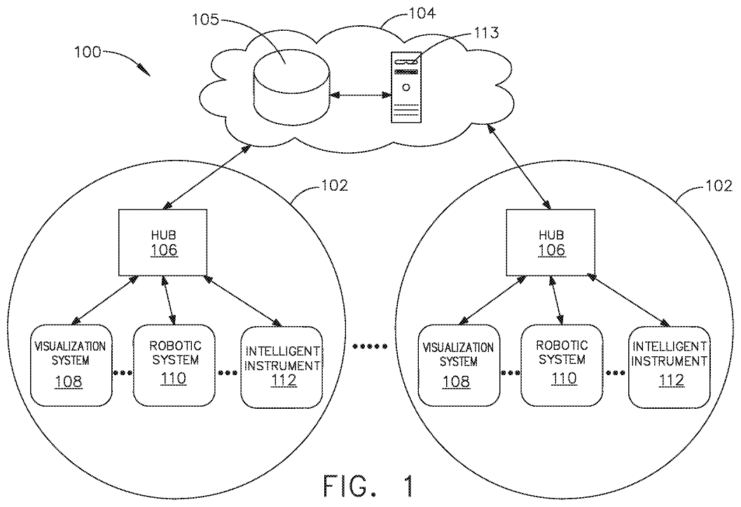

Referring to FIG. 1, a computer-implemented interactive surgical system 100 includes one or more surgical systems 102 and a cloud-based system (e.g., the cloud 104 that may include a remote server 113 coupled to a storage device 105). Each surgical system 102 includes at least one surgical hub 106 in communication with the cloud 104 that may include a remote server 113. In one example, as illustrated in FIG. 1, the surgical system 102 includes a visualization system 108, a robotic system 110, and a handheld intelligent surgical instrument 112, which are configured to communicate with one another and/or the hub 106. In some aspects, a surgical system 102 may include an M number of hubs 106, an N number of visualization systems 108, an O number of robotic systems 110, and a P number of handheld intelligent surgical instruments 112, where M, N, O, and P are integers greater than or equal to one.

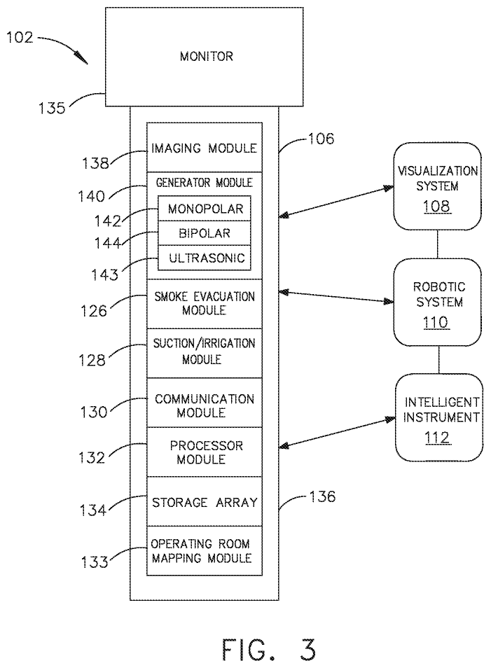

FIG. 3 depicts an example of a surgical system 102 being used to perform a surgical procedure on a patient who is lying down on an operating table 114 in a surgical operating room 116. A robotic system 110 is used in the surgical procedure as a part of the surgical system 102. The robotic system 110 includes a surgeon's console 118, a patient side cart 120 (surgical robot), and a surgical robotic hub 122. The patient side cart 120 can manipulate at least one removably coupled surgical tool 117 through a minimally invasive incision in the body of the patient while the surgeon views the surgical site through the surgeon's console 118. An image of the surgical site can be obtained by a medical imaging device 124, which can be manipulated by the patient side cart 120 to orient the imaging device 124. The robotic hub 122 can be used to process the images of the surgical site for subsequent display to the surgeon through the surgeon's console 118.

Other types of robotic systems can be readily adapted for use with the surgical system 102. Various examples of robotic systems and surgical tools that are suitable for use with the present disclosure are described in U.S. Provisional Patent Application Ser. No. 62/611,339, titled ROBOT ASSISTED SURGICAL PLATFORM, filed Dec. 28, 2017, the disclosure of which is herein incorporated by reference in its entirety.

Various examples of cloud-based analytics that are performed by the cloud 104, and are suitable for use with the present disclosure, are described in U.S. Provisional Patent Application Ser. No. 62/611,340, titled CLOUD-BASED MEDICAL ANALYTICS, filed Dec. 28, 2017, the disclosure of which is herein incorporated by reference in its entirety.

In various aspects, the imaging device 124 includes at least one image sensor and one or more optical components. Suitable image sensors include, but are not limited to, Charge-Coupled Device (CCD) sensors and Complementary Metal-Oxide Semiconductor (CMOS) sensors.

The optical components of the imaging device 124 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is that portion of the electromagnetic spectrum that is visible to (i.e., can be detected by) the human eye and may be referred to as visible light or simply light. A typical human eye will respond to wavelengths in air that are from about 380 nm to about 750 nm.

The invisible spectrum (i.e., the non-luminous spectrum) is that portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 124 is configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

In one aspect, the imaging device employs multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information the human eye fails to capture with its receptors for red, green, and blue. The use of multi-spectral imaging is described in greater detail under the heading “Advanced Imaging Acquisition Module” in U.S. Provisional Patent Application Ser. No. 62/611,341, titled INTERACTIVE SURGICAL PLATFORM, filed Dec. 28, 2017, the disclosure of which is herein incorporated by reference in its entirety. Multi-spectrum monitoring can be a useful tool in relocating a surgical field after a surgical task is completed to perform one or more of the previously described tests on the treated tissue.

It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device 124 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

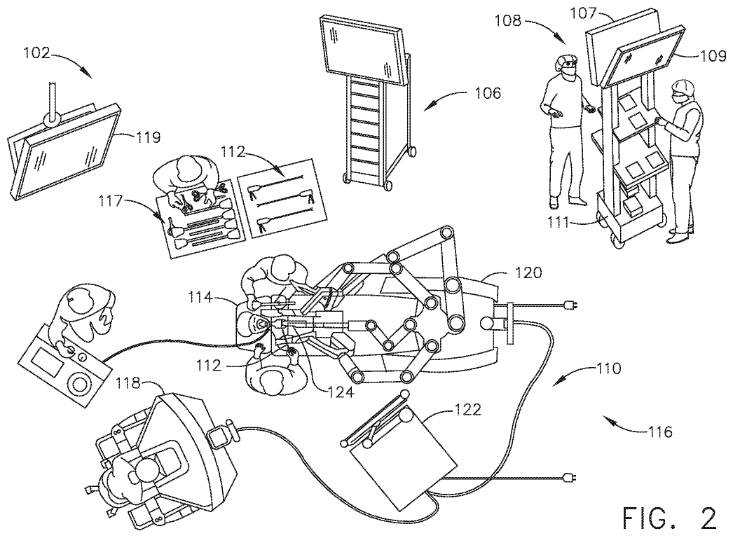

In various aspects, the visualization system 108 includes one or more imaging sensors, one or more image processing units, one or more storage arrays, and one or more displays that are strategically arranged with respect to the sterile field, as illustrated in FIG. 2. In one aspect, the visualization system 108 includes an interface for HL7, PACS, and EMR. Various components of the visualization system 108 are described under the heading “Advanced Imaging Acquisition Module” in U.S. Provisional Patent Application Ser. No. 62/611,341, titled INTERACTIVE SURGICAL PLATFORM, filed Dec. 28, 2017, the disclosure of which is herein incorporated by reference in its entirety.

As illustrated in FIG. 2, a primary display 119 is positioned in the sterile field to be visible to an operator at the operating table 114. In addition, a visualization tower 111 is positioned outside the sterile field. The visualization tower 111 includes a first non-sterile display 107 and a second non-sterile display 109, which face away from each other. The visualization system 108, guided by the hub 106, is configured to utilize the displays 107, 109, and 119 to coordinate information flow to operators inside and outside the sterile field. For example, the hub 106 may cause the visualization system 108 to display a snap-shot of a surgical site, as recorded by an imaging device 124, on a non-sterile display 107 or 109, while maintaining a live feed of the surgical site on the primary display 119. The snap-shot on the non-sterile display 107 or 109 can permit a non-sterile operator to perform a diagnostic step relevant to the surgical procedure, for example.

In one aspect, the hub 106 is also configured to route a diagnostic input or feedback entered by a non-sterile operator at the visualization tower 111 to the primary display 119 within the sterile field, where it can be viewed by a sterile operator at the operating table. In one example, the input can be in the form of a modification to the snap-shot displayed on the non-sterile display 107 or 109, which can be routed to the primary display 119 by the hub 106.

Referring to FIG. 2, a surgical instrument 112 is being used in the surgical procedure as part of the surgical system 102. The hub 106 is also configured to coordinate information flow to a display of the surgical instrument 112. For example, in U.S. Provisional Patent Application Ser. No. 62/611,341, titled INTERACTIVE SURGICAL PLATFORM, filed Dec. 28, 2017, the disclosure of which is herein incorporated by reference in its entirety. A diagnostic input or feedback entered by a non-sterile operator at the visualization tower 111 can be routed by the hub 106 to the surgical instrument display 115 within the sterile field, where it can be viewed by the operator of the surgical instrument 112. Example surgical instruments that are suitable for use with the surgical system 102 are described under the heading “Surgical Instrument Hardware” and in U.S. Provisional Patent Application Ser. No. 62/611,341, titled INTERACTIVE SURGICAL PLATFORM, filed Dec. 28, 2017, the disclosure of which is herein incorporated by reference in its entirety, for example.

Referring now to FIG. 3, a hub 106 is depicted in communication with a visualization system 108, a robotic system 110, and a handheld intelligent surgical instrument 112. The hub 106 includes a hub display 135, an imaging module 138, a generator module 140, a communication module 130, a processor module 132, and a storage array 134. In certain aspects, as illustrated in FIG. 3, the hub 106 further includes a smoke evacuation module 126 and/or a suction/irrigation module 128.

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure 136 offers a unified environment for managing the power, data, and fluid lines, which reduces the frequency of entanglement between such lines.

Aspects of the present disclosure present a surgical hub for use in a surgical procedure that involves energy application to tissue at a surgical site. The surgical hub includes a hub enclosure and a combo generator module slidably receivable in a docking station of the hub enclosure. The docking station includes data and power contacts. The combo generator module includes two or more of an ultrasonic energy generator component, a bipolar RF energy generator component, and a monopolar RF energy generator component that are housed in a single unit. In one aspect, the combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component.

In one aspect, the fluid line is a first fluid line and a second fluid line extends from the remote surgical site to a suction and irrigation module slidably received in the hub enclosure. In one aspect, the hub enclosure comprises a fluid interface.

Certain surgical procedures may require the application of more than one energy type to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 136 is configured to accommodate different generators, and facilitate an interactive communication therebetween. One of the advantages of the hub modular enclosure 136 is enabling the quick removal and/or replacement of various modules.

Aspects of the present disclosure present a modular surgical enclosure for use in a surgical procedure that involves energy application to tissue. The modular surgical enclosure includes a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts.

Further to the above, the modular surgical enclosure also includes a second energy-generator module configured to generate a second energy, different than the first energy, for application to the tissue, and a second docking station comprising a second docking port that includes second data and power contacts, wherein the second energy-generator module is slidably movable into an electrical engagement with the power and data contacts, and wherein the second energy-generator module is slidably movable out of the electrical engagement with the second power and data contacts.

In addition, the modular surgical enclosure also includes a communication bus between the first docking port and the second docking port, configured to facilitate communication between the first energy-generator module and the second energy-generator module.

Referring to FIG. 3, aspects of the present disclosure are presented for a hub modular enclosure 136 that allows the modular integration of a generator module 140, a smoke evacuation module 126, and a suction/irrigation module 128. The hub modular enclosure 136 further facilitates interactive communication between the modules 140, 126, 128. The generator module 140 can be a generator module with integrated monopolar, bipolar, and ultrasonic components supported in a single housing unit slidably insertable into the hub modular enclosure 136. In various aspects, the hub modular enclosure 136 can be configured to facilitate the insertion of multiple generators and interactive communication between the generators docked into the hub modular enclosure 136 so that the generators would act as a single generator.

In one aspect, the hub modular enclosure 136 comprises a modular power and communication backplane with external and wireless communication headers to enable the removable attachment of the modules 140, 126, 128 and interactive communication therebetween.

In various aspects, the imaging module 138 comprises an integrated video processor and a modular light source and is adapted for use with various imaging devices. In one aspect, the imaging device is comprised of a modular housing that can be assembled with a light source module and a camera module. The housing can be a disposable housing. In at least one example, the disposable housing is removably coupled to a reusable controller, a light source module, and a camera module. The light source module and/or the camera module can be selectively chosen depending on the type of surgical procedure. In one aspect, the camera module comprises a CCD sensor. In another aspect, the camera module comprises a CMOS sensor. In another aspect, the camera module is configured for scanned beam imaging. Likewise, the light source module can be configured to deliver a white light or a different light, depending on the surgical procedure.

During a surgical procedure, removing a surgical device from the surgical field and replacing it with another surgical device that includes a different camera or a different light source can be inefficient. Temporarily losing sight of the surgical field may lead to undesirable consequences. The module imaging device of the present disclosure is configured to permit the replacement of a light source module or a camera module midstream during a surgical procedure, without having to remove the imaging device from the surgical field.

In one aspect, the imaging device comprises a tubular housing that includes a plurality of channels. A first channel is configured to slidably receive the camera module, which can be configured for a snap-fit engagement with the first channel. A second channel is configured to slidably receive the light source module, which can be configured for a snap-fit engagement with the second channel. In another example, the camera module and/or the light source module can be rotated into a final position within their respective channels. A threaded engagement can be employed in lieu of the snap-fit engagement.

In various examples, multiple imaging devices are placed at different positions in the surgical field to provide multiple views. The imaging module 138 can be configured to switch between the imaging devices to provide an optimal view. In various aspects, the imaging module 138 can be configured to integrate the images from the different imaging device.

Various image processors and imaging devices suitable for use with the present disclosure are described in U.S. Pat. No. 7,995,045, titled COMBINED SBI AND CONVENTIONAL IMAGE PROCESSOR, which issued on Aug. 9, 2011, which is herein incorporated by reference in its entirety. In addition, U.S. Pat. No. 7,982,776, titled SBI MOTION ARTIFACT REMOVAL APPARATUS AND METHOD, which issued on Jul. 19, 2011, which is herein incorporated by reference in its entirety, describes various systems for removing motion artifacts from image data. Such systems can be integrated with the imaging module 138. Furthermore, U.S. Patent Application Publication No. 2011/0306840, titled CONTROLLABLE MAGNETIC SOURCE TO FIXTURE INTRACORPOREAL APPARATUS, which published on Dec. 15, 2011, and U.S. Patent Application Publication No. 2014/0243597, titled SYSTEM FOR PERFORMING A MINIMALLY INVASIVE SURGICAL PROCEDURE, which published on Aug. 28, 2014, each of which is herein incorporated by reference in its entirety.

Robotic Surgical System

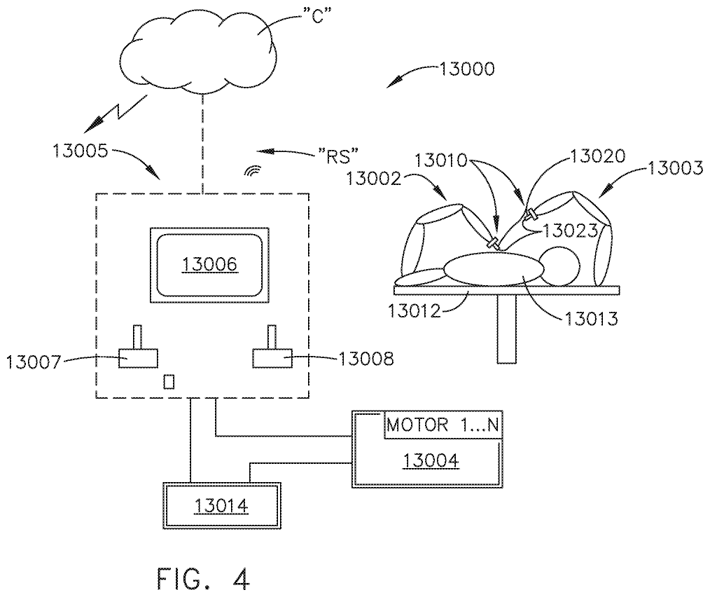

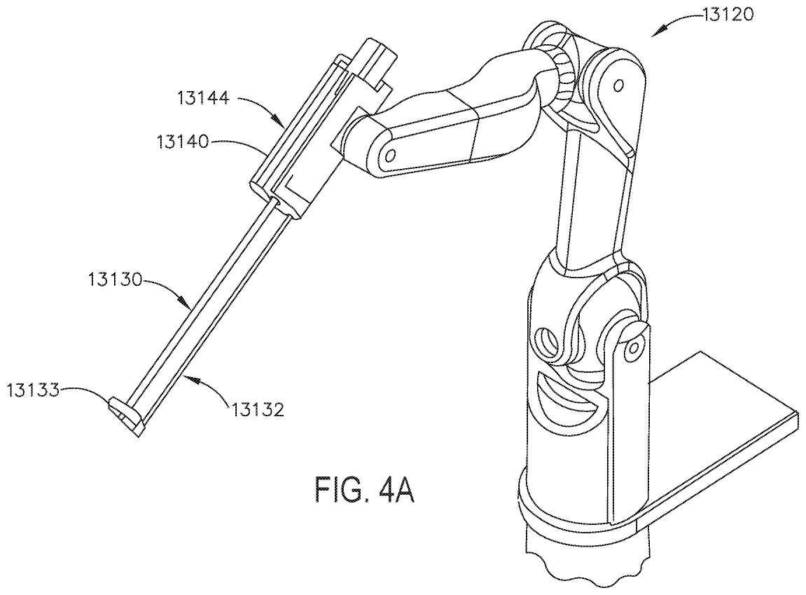

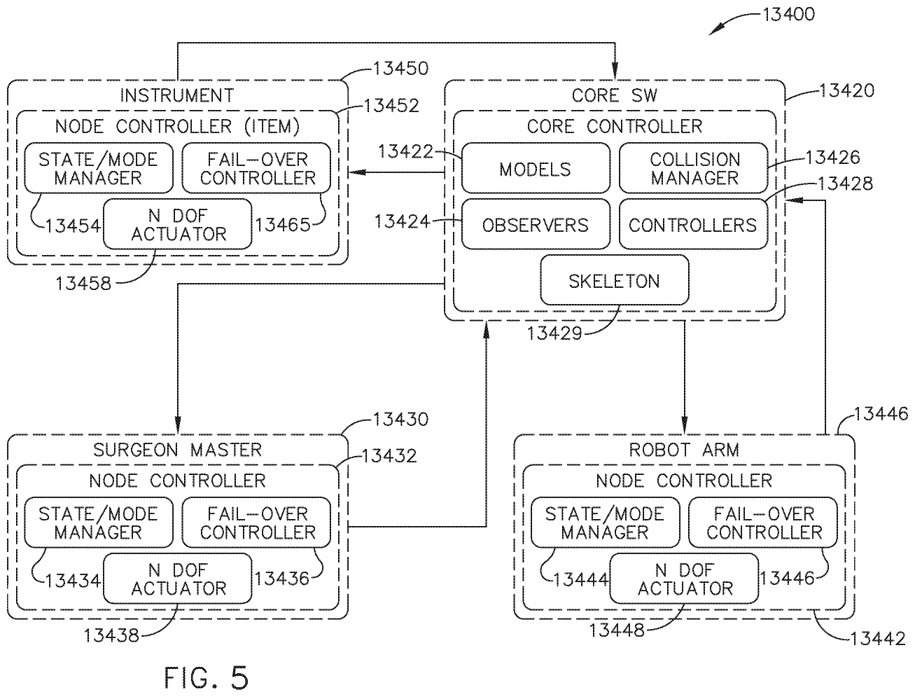

An example robotic surgical system is depicted in FIGS. 4 and 5. With reference to FIG. 4, the robotic surgical system 13000 includes robotic arms 13002, 13003, a control device 13004, and a console 13005 coupled to the control device 13004. As illustrated in FIG. 4, the surgical system 13000 is configured for use on a patient 13013 lying on a patient table 13012 for performance of a minimally invasive surgical operation. The console 13005 includes a display device 13006 and input devices 13007, 13008. The display device 13006 is set up to display three-dimensional images, and the manual input devices 13007, 13008 are configured to allow a clinician to telemanipulate the robotic arms 13002, 13003. Controls for a surgeon's console, such as the console 13005, are further described in International Patent Publication No. WO2017/075121, filed Oct. 27, 2016, titled HAPTIC FEEDBACK FOR A ROBOTIC SURGICAL SYSTEM INTERFACE, which is herein incorporated by reference in its entirety.

Each of the robotic arms 13002, 13003 is made up of a plurality of members connected through joints and includes a surgical assembly 13010 connected to a distal end of a corresponding robotic arm 13002, 13003. Support of multiple arms is further described in U.S. Patent Application Publication No. 2017/0071693, filed Nov. 11, 2016, titled SURGICAL ROBOTIC ARM SUPPORT SYSTEMS AND METHODS OF USE, which is herein incorporated by reference in its entirety. Various robotic arm configurations are further described in International Patent Publication No. WO2017/044406, filed Sep. 6, 2016, titled ROBOTIC SURGICAL CONTROL SCHEME FOR MANIPULATING ROBOTIC END EFFECTORS, which is herein incorporated by reference in its entirety. In an exemplification, the surgical assembly 13010 includes a surgical instrument 13020 supporting an end effector 13023. Although two robotic arms 13002, 13003, are depicted, the surgical system 13000 may include a single robotic arm or more than two robotic arms 13002, 13003. Additional robotic arms are likewise connected to the control device 13004 and are telemanipulatable via the console 13005. Accordingly, one or more additional surgical assemblies 13010 and/or surgical instruments 13020 may also be attached to the additional robotic arm(s).

The robotic arms 13002, 13003 may be driven by electric drives that are connected to the control device 13004. According to an exemplification, the control device 13004 is configured to activate drives, for example, via a computer program, such that the robotic arms 13002, 13003 and the surgical assemblies 13010 and/or surgical instruments 13020 corresponding to the robotic arms 13002, 13003, execute a desired movement received through the manual input devices 13007, 13008. The control device 13004 may also be configured to regulate movement of the robotic arms 13002, 13003 and/or of the drives.

The control device 13004 may control a plurality of motors (for example, Motor I . . . n) with each motor configured to drive a pushing or a pulling of one or more cables, such as cables coupled to the end effector 13023 of the surgical instrument 13020. In use, as these cables are pushed and/or pulled, the one or more cables affect operation and/or movement of the end effector 13023. The control device 13004 coordinates the activation of the various motors to coordinate a pushing or a pulling motion of one or more cables in order to coordinate an operation and/or movement of one or more end effectors 13023. For example, articulation of an end effector by a robotic assembly such as the surgical assembly 13010 is further described in U.S. Patent Application Publication No. 2016/0303743, filed Jun. 6, 2016, titled WRIST AND JAW ASSEMBLIES FOR ROBOTIC SURGICAL SYSTEMS and in International Patent Publication No. WO2016/144937, filed Mar. 8, 2016, titled MEASURING HEALTH OF A CONNECTOR MEMBER OF A ROBOTIC SURGICAL SYSTEM, each of which is herein incorporated by reference in its entirety. In an exemplification, each motor is configured to actuate a drive rod or a lever arm to affect operation and/or movement of end effectors 13023 in addition to, or instead of, one or more cables.

Driver configurations for surgical instruments, such as drive arrangements for a surgical end effector, are further described in International Patent Publication No. WO2016/183054, filed May 10, 2016, titled COUPLING INSTRUMENT DRIVE UNIT AND ROBOTIC SURGICAL INSTRUMENT, International Patent Publication No. WO2016/205266, filed Jun. 15, 2016, titled ROBOTIC SURGICAL SYSTEM TORQUE TRANSDUCTION SENSING, International Patent Publication No. WO2016/205452, filed Jun. 16, 2016, titled CONTROLLING ROBOTIC SURGICAL INSTRUMENTS WITH BIDIRECTIONAL COUPLING, and International Patent Publication No. WO2017/053507, filed Sep. 22, 2016, titled ELASTIC SURGICAL INTERFACE FOR ROBOTIC SURGICAL SYSTEMS, each of which is herein incorporated by reference in its entirety. The modular attachment of surgical instruments to a driver is further described in International Patent Publication No. WO2016/209769, filed Jun. 20, 2016, titled ROBOTIC SURGICAL ASSEMBLIES, which is herein incorporated by reference in its entirety. Housing configurations for a surgical instrument driver and interface are further described in International Patent Publication No. WO2016/144998, filed Mar. 9, 2016, titled ROBOTIC SURGICAL SYSTEMS, INSTRUMENT DRIVE UNITS, AND DRIVE ASSEMBLIES, which is herein incorporated by reference in its entirety. Various surgical instrument configurations for use with the robotic arms 13002, 13003 are further described in International Patent Publication No. WO2017/053358, filed Sep. 21, 2016, titled SURGICAL ROBOTIC ASSEMBLIES AND INSTRUMENT ADAPTERS THEREOF and International Patent Publication No. WO2017/053363, filed Sep. 21, 2016, titled ROBOTIC SURGICAL ASSEMBLIES AND INSTRUMENT DRIVE CONNECTORS THEREOF, each of which is herein incorporated by reference in its entirety. Bipolar instrument configurations for use with the robotic arms 13002, 13003 are further described in International Patent Publication No. WO2017/053698, filed Sep. 23, 2016, titled ROBOTIC SURGICAL ASSEMBLIES AND ELECTROMECHANICAL INSTRUMENTS THEREOF, which is herein incorporated by reference in its entirety. Shaft arrangements for use with the robotic arms 13002, 13003 are further described in International Patent Publication No. WO2017/116793, filed Dec. 19, 2016, titled ROBOTIC SURGICAL SYSTEMS AND INSTRUMENT DRIVE ASSEMBLIES, which is herein incorporated by reference in its entirety.

The control device 13004 includes any suitable logic control circuit adapted to perform calculations and/or operate according to a set of instructions. The control device 13004 can be configured to communicate with a remote system “RS,” either via a wireless (e.g., Wi-Fi, Bluetooth, LTE, etc.) and/or wired connection. The remote system “RS” can include data, instructions and/or information related to the various components, algorithms, and/or operations of system 13000. The remote system “RS” can include any suitable electronic service, database, platform, cloud “C” (see FIG. 4), or the like. The control device 13004 may include a central processing unit operably connected to memory. The memory may include transitory type memory (e.g., RAM) and/or non-transitory type memory (e.g., flash media, disk media, etc.). In some exemplifications, the memory is part of, and/or operably coupled to, the remote system “RS.”

The control device 13004 can include a plurality of inputs and outputs for interfacing with the components of the system 13000, such as through a driver circuit. The control device 13004 can be configured to receive input signals and/or generate output signals to control one or more of the various components (e.g., one or more motors) of the system 13000. The output signals can include, and/or can be based upon, algorithmic instructions which may be pre-programmed and/or input by a user. The control device 13004 can be configured to accept a plurality of user inputs from a user interface (e.g., switches, buttons, touch screen, etc. of operating the console 13005) which may be coupled to remote system “RS.”

A memory 13014 can be directly and/or indirectly coupled to the control device 13004 to store instructions and/or databases including pre-operative data from living being(s) and/or anatomical atlas(es). The memory 13014 can be part of, and/or or operatively coupled to, remote system “RS.”

In accordance with an exemplification, the distal end of each robotic arm 13002, 13003 is configured to releasably secure the end effector 13023 (or other surgical tool) therein and may be configured to receive any number of surgical tools or instruments, such as a trocar or retractor, for example.

A simplified functional block diagram of a system architecture 13400 of the robotic surgical system 13000 is depicted in FIG. 5. The system architecture 13400 includes a core module 13420, a surgeon master module 13430, a robotic arm module 13440, and an instrument module 13450. The core module 13420 serves as a central controller for the robotic surgical system 13000 and coordinates operations of all of the other modules 13430, 13440, 13450. For example, the core module 13420 maps control devices to the arms 13002, 13003, determines current status, performs all kinematics and frame transformations, and relays resulting movement commands. In this regard, the core module 13420 receives and analyzes data from each of the other modules 13430, 13440, 13450 in order to provide instructions or commands to the other modules 13430, 13440, 13450 for execution within the robotic surgical system 13000. Although depicted as separate modules, one or more of the modules 13420, 13430, 13440, and 13450 are a single component in other exemplifications.

The core module 13420 includes models 13422, observers 13424, a collision manager 13426, controllers 13428, and a skeleton 13429. The models 13422 include units that provide abstracted representations (base classes) for controlled components, such as the motors (for example, Motor I . . . n) and/or the arms 13002, 13003. The observers 13424 create state estimates based on input and output signals received from the other modules 13430, 13440, 13450. The collision manager 13426 prevents collisions between components that have been registered within the system 13000. The skeleton 13429 tracks the system 13000 from a kinematic and dynamics point of view. For example, the kinematics item may be implemented either as forward or inverse kinematics, in an exemplification. The dynamics item may be implemented as algorithms used to model dynamics of the system's components.

The surgeon master module 13430 communicates with surgeon control devices at the console 13005 and relays inputs received from the console 13005 to the core module 13420. In accordance with an exemplification, the surgeon master module 13430 communicates button status and control device positions to the core module 13420 and includes a node controller 13432 that includes a state/mode manager 13434, a fail-over controller 13436, and a N-degree of freedom (“DOF”) actuator 13438.

The robotic arm module 13440 coordinates operation of a robotic arm subsystem, an arm cart subsystem, a set up arm, and an instrument subsystem in order to control movement of a corresponding arm 13002, 13003. Although a single robotic arm module 13440 is included, it will be appreciated that the robotic arm module 13440 corresponds to and controls a single arm. As such, additional robotic arm modules 13440 are included in configurations in which the system 13000 includes multiple arms 13002, 13003. The robotic arm module 13440 includes a node controller 13442, a state/mode manager 13444, a fail-over controller 13446, and a N-degree of freedom (“DOF”) actuator 13348.

The instrument module 13450 controls movement of an instrument and/or tool component attached to the arm 13002, 13003. The instrument module 13450 is configured to correspond to and control a single instrument. Thus, in configurations in which multiple instruments are included, additional instrument modules 13450 are likewise included. In an exemplification, the instrument module 13450 obtains and communicates data related to the position of the end effector or jaw assembly (which may include the pitch and yaw angle of the jaws), the width of or the angle between the jaws, and the position of an access port. The instrument module 13450 has a node controller 13452, a state/mode manager 13454, a fail-over controller 13456, and a N-degree of freedom (“DOF”) actuator 13458.

The position data collected by the instrument module 13450 is used by the core module 13420 to determine when the instrument is within the surgical site, within a cannula, adjacent to an access port, or above an access port in free space. The core module 13420 can determine whether to provide instructions to open or close the jaws of the instrument based on the positioning thereof. For example, when the position of the instrument indicates that the instrument is within a cannula, instructions are provided to maintain a jaw assembly in a closed position. When the position of the instrument indicates that the instrument is outside of an access port, instructions are provided to open the jaw assembly.

Additional features and operations of a robotic surgical system, such as the surgical robot system depicted in FIGS. 4 and 5, are further described in the following references, each of which is herein incorporated by reference in its entirety:
U.S. Patent Application Publication No. 2016/0303743, filed Jun. 6, 2016, titled WRIST AND JAW ASSEMBLIES FOR ROBOTIC SURGICAL SYSTEMS;
U.S. Patent Application Publication No. 2017/0071693, filed Nov. 11, 2016, titled SURGICAL ROBOTIC ARM SUPPORT SYSTEMS AND METHODS OF USE;
International Patent Publication No. WO2016/144937, filed Mar. 8, 2016, titled MEASURING HEALTH OF A CONNECTOR MEMBER OF A ROBOTIC SURGICAL SYSTEM;
International Patent Publication No. WO2016/144998, filed Mar. 9, 2016, titled ROBOTIC SURGICAL SYSTEMS, INSTRUMENT DRIVE UNITS, AND DRIVE ASSEMBLIES;
International Patent Publication No. WO2016/183054, filed May 10, 2016, titled COUPLING INSTRUMENT DRIVE UNIT AND ROBOTIC SURGICAL INSTRUMENT;
International Patent Publication No. WO2016/205266, filed Jun. 15, 2016, titled ROBOTIC SURGICAL SYSTEM TORQUE TRANSDUCTION SENSING;
International Patent Publication No. WO2016/205452, filed Jun. 16, 2016, titled CONTROLLING ROBOTIC SURGICAL INSTRUMENTS WITH BIDIRECTIONAL COUPLING;
International Patent Publication No. WO2016/209769, filed Jun. 20, 2016, titled ROBOTIC SURGICAL ASSEMBLIES;
International Patent Publication No. WO2017/044406, filed Sep. 6, 2016, titled ROBOTIC SURGICAL CONTROL SCHEME FOR MANIPULATING ROBOTIC END EFFECTORS;
International Patent Publication No. WO2017/053358, filed Sep. 21, 2016, titled SURGICAL ROBOTIC ASSEMBLIES AND INSTRUMENT ADAPTERS THEREOF;
International Patent Publication No. WO2017/053363, filed Sep. 21, 2016, titled ROBOTIC SURGICAL ASSEMBLIES AND INSTRUMENT DRIVE CONNECTORS THEREOF;
International Patent Publication No. WO2017/053507, filed Sep. 22, 2016, titled ELASTIC SURGICAL INTERFACE FOR ROBOTIC SURGICAL SYSTEMS;
International Patent Publication No. WO2017/053698, filed Sep. 23, 2016, titled ROBOTIC SURGICAL ASSEMBLIES AND ELECTROMECHANICAL INSTRUMENTS THEREOF;
International Patent Publication No. WO2017/075121, filed Oct. 27, 2016, titled HAPTIC FEEDBACK CONTROLS FOR A ROBOTIC SURGICAL SYSTEM INTERFACE;
International Patent Publication No. WO2017/116793, filed Dec. 19, 2016, titled ROBOTIC SURGICAL SYSTEMS AND INSTRUMENT DRIVE ASSEMBLIES.

The robotic surgical systems and features disclosed herein can be employed with the robotic surgical system of FIGS. 4 and 5. The reader will further appreciate that various systems and/or features disclosed herein can also be employed with alternative surgical systems including the computer-implemented interactive surgical system 100, the computer-implemented interactive surgical system 200, the robotic surgical system 110, the robotic hub 122, and/or the robotic hub 222, for example.

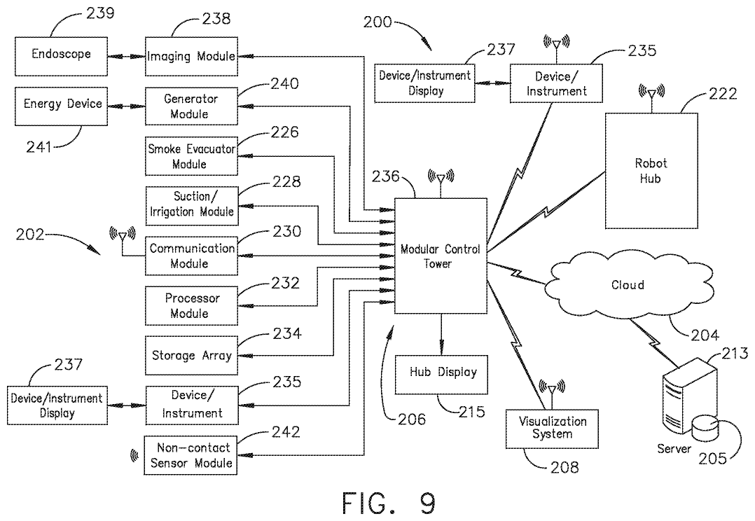

In various instances, a robotic surgical system can include a robotic control tower, which can house the control unit of the system. For example, the control unit 13004 of the robotic surgical system 13000 (FIG. 4) can be housed within a robotic control tower. The robotic control tower can include a robotic hub such as the robotic hub 122 (FIG. 2) or the robotic hub 222 (FIG. 9), for example. Such a robotic hub can include a modular interface for coupling with one or more generators, such as an ultrasonic generator and/or a radio frequency generator, and/or one or more modules, such as an imaging module, suction module, an irrigation module, a smoke evacuation module, and/or a communication module.

A robotic hub can include a situational awareness module, which can be configured to synthesize data from multiple sources to determine an appropriate response to a surgical event. For example, a situational awareness module can determine the type of surgical procedure, step in the surgical procedure, type of tissue, and/or tissue characteristics, as further described herein. Moreover, such a module can recommend a particular course of action or possible choices to the robotic system based on the synthesized data. In various instances, a sensor system encompassing a plurality of sensors distributed throughout the robotic system can provide data, images, and/or other information to the situational awareness module. Such a situational awareness module can be incorporated into a control unit, such as the control unit 13004, for example. In various instances, the situational awareness module can obtain data and/or information from a non-robotic surgical hub and/or a cloud, such as the surgical hub 106 (FIG. 1), the surgical hub 206 (FIG. 10), the cloud 104 (FIG. 1), and/or the cloud 204 (FIG. 9), for example. Situational awareness of a surgical system is further disclosed herein and in U.S. Provisional Patent Application Ser. No. 62/611,341, titled INTERACTIVE SURGICAL PLATFORM, filed Dec. 28, 2017, and U.S. Provisional Patent Application Ser. No. 62/611,340, titled CLOUD-BASED MEDICAL ANALYTICS, filed Dec. 28, 2017, the disclosure of each of which is herein incorporated by reference in its entirety.

In certain instances, the activation of a surgical tool at certain times during a surgical procedure and/or for certain durations may cause tissue trauma and/or may prolong a surgical procedure. For example, a robotic surgical system can utilize an electrosurgical tool having an energy delivery surface that should only be energized when a threshold condition is met. In one example, the energy delivery surface should only be activated when the energy delivery surface is in contact with the appropriate, or targeted, tissue. As another example, a robotic surgical system can utilize a suction element that should only be activated when a threshold condition is met, such as when an appropriate volume of fluid is present. Due to visibility restrictions, evolving situations, and the multitude of moving parts during a robotic surgical procedure, it can be difficult for a clinician to determine and/or monitor certain conditions at the surgical site. For example, it can be difficult to determine if an energy delivery surface of an electrosurgical tool is in contact with tissue. It can also be difficult to determine if a particular suctioning pressure is sufficient for the volume of fluid in the proximity of the suctioning port.

Moreover, a plurality of surgical devices can be used in certain robotic surgical procedures. For example, a robotic surgical system can use one or more surgical tools during the surgical procedure. Additionally, one or more handheld instruments can also be used during the surgical procedure. One or more of the surgical devices can include a sensor. For example, multiple sensors can be positioned around the surgical site and/or the operating room. A sensor system including the one or more sensors can be configured to detect one or more conditions at the surgical site. For example, data from the sensor system can determine if a surgical tool mounted to the surgical robot is being used and/or if a feature of the surgical tool should be activated. More specifically, a sensor system can detect if an electrosurgical device is positioned in abutting contact with tissue, for example. As another example, a sensor system can detect if a suctioning element of a surgical tool is applying a sufficient suctioning force to fluid at the surgical site.

When in an automatic activation mode, the robotic surgical system can automatically activate one or more features of one or more surgical tools based on data, images, and/or other information received from the sensor system. For example, an energy delivery surface of an electrosurgical tool can be activated upon detecting that the electrosurgical tool is in use (e.g. positioned in abutting contact with tissue). As another example, a suctioning element on a surgical tool can be activated when the suction port is moved into contact with a fluid. In certain instances, the surgical tool can be adjusted based on the sensed conditions.

A robotic surgical system incorporating an automatic activation mode can automatically provide a scenario-specific result based on detected condition(s) at the surgical site. The scenario-specific result can be outcome-based, for example, and can streamline the decision-making process of the clinician. In certain instances, such an automatic activation mode can improve the efficiency and/or effectiveness of the clinician. For example, the robotic surgical system can aggregate data to compile a more complete view of the surgical site and/or the surgical procedure in order to determine the best possible course of action. Additionally or alternatively, in instances in which the clinician makes fewer decisions, the clinician can be better focused on other tasks and/or can process other information more effectively.

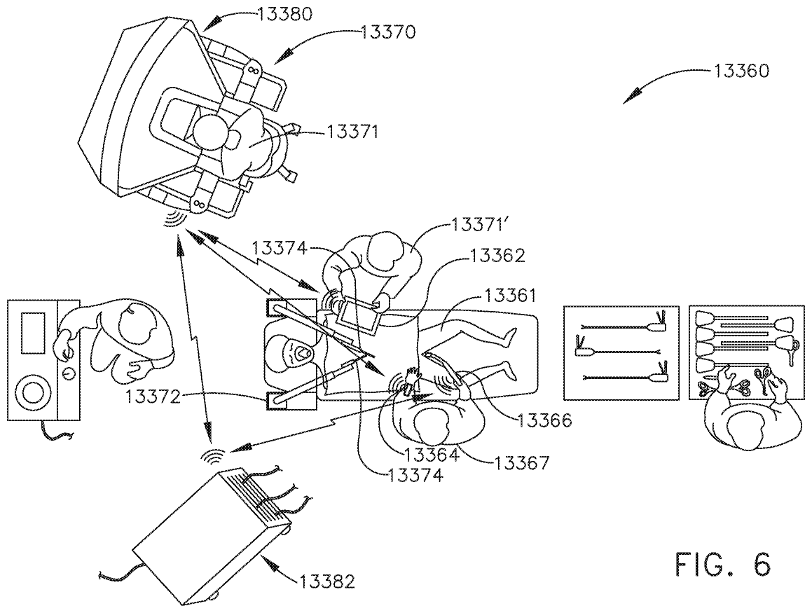

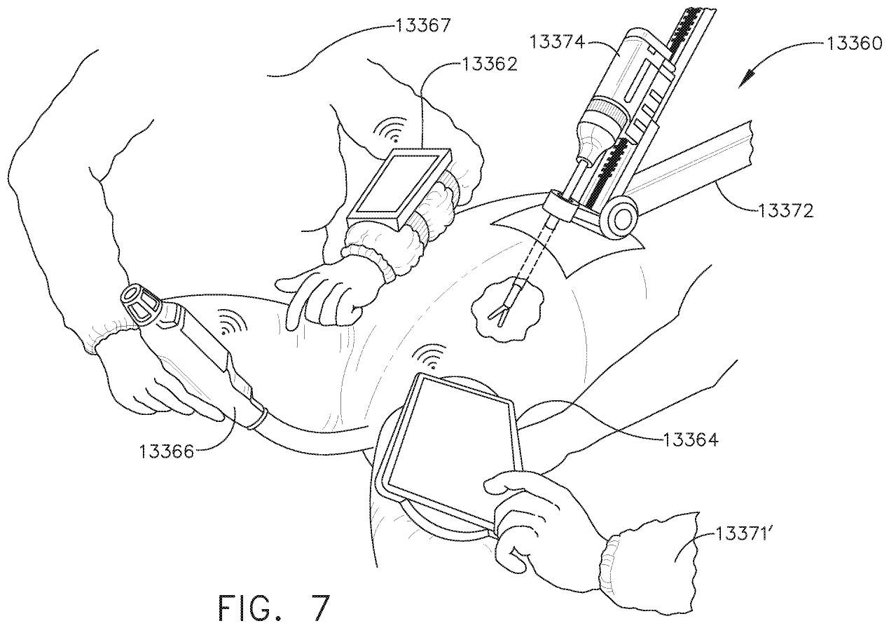

Referring primarily to FIGS. 6 and 7, hubs 13380, 13382 include wireless communication modules such that a wireless communication link is established between the two hubs 13380, 13382. Additionally, the robotic hub 13380 is in signal communication with the interactive secondary displays 13362, 13364 within the sterile field. The hub 13382 is in signal communication with the handheld surgical instrument 13366. If the surgeon 13371 moves over towards the patient 13361 and within the sterile field (as indicated by the reference character 13371′), the surgeon 13371 can use one of the wireless interactive displays 13362, 13364 to operate the robot 13372 away from the remote command console 13370. The plurality of secondary displays 13362, 13364 within the sterile field allows the surgeon 13371 to move away from the remote command console 13370 without losing sight of important information for the surgical procedure and controls for the robotic tools utilized therein.

The interactive secondary displays 13362, 13364 permit the clinician to step away from the remote command console 13370 and into the sterile field while maintaining control of the robot 13372. For example, the interactive secondary displays 13362, 13364 allow the clinician to maintain cooperative and/or coordinated control over the powered handheld surgical instrument(s) 13366 and the robotic surgical system at the same time. In various instances, information is communicated between the robotic surgical system, one or more powered handheld surgical instruments 13366, surgical hubs 13380, 13382, and the interactive secondary displays 13362, 13364. Such information may include, for example, the images on the display of the robotic surgical system and/or the powered handheld surgical instruments, a parameter of the robotic surgical system and/or the powered handheld surgical instruments, and/or a control command for the robotic surgical system and/or the powered handheld surgical instruments.

In various instances, the control unit of the robotic surgical system (e.g. the control unit 13113 of the robotic surgical system 13110) is configured to communicate at least one display element from the surgeon's command console (e.g. the console 13116) to an interactive secondary display (e.g. the displays 13362, 13364). In other words, a portion of the display at the surgeon's console is replicated on the display of the interactive secondary display, integrating the robot display with the interactive secondary display. The replication of the robot display on to the display of the interactive secondary display allows the clinician to step away from the remote command console without losing the visual image that is displayed there. For example, at least one of the interactive secondary displays 13362, 13364 can display information from the robot, such as information from the robot display and/or the surgeon's command console 13370.

In various instances, the interactive secondary displays 13362, 13364 are configured to control and/or adjust at least one operating parameter of the robotic surgical system. Such control can occur automatically and/or in response to a clinician input. Interacting with a touch-sensitive screen and/or buttons on the interactive secondary display(s) 13362, 13364, the clinician is able to input a command to control movement and/or functionality of the one or more robotic tools. For example, when utilizing a handheld surgical instrument 13366, the clinician may want to move the robotic tool 13374 to a different position. To control the robotic tool 13374, the clinician applies an input to the interactive secondary display(s) 13362, 13364, and the respective interactive secondary display(s) 13362, 13364 communicates the clinician input to the control unit of the robotic surgical system in the robotic hub 13380.

In various instances, a clinician positioned at the remote command console 13370 of the robotic surgical system can manually override any robot command initiated by a clinician input on the one or more interactive secondary displays 13362, 13364. For example, when a clinician input is received from the one or more interactive secondary displays 13362, 13364, a clinician positioned at the remote command console 13370 can either allow the command to be issued and the desired function performed or the clinician can override the command by interacting with the remote command console 13370 and prohibiting the command from being issued.

In certain instances, a clinician within the sterile field can be required to request permission to control the robot 13372 and/or the robotic tool 13374 mounted thereto. The surgeon 13371 at the remote command console 13370 can grant or deny the clinician's request. For example, the surgeon can receive a pop-up or other notification indicating the permission is being requested by another clinician operating a handheld surgical instrument and/or interacting with an interactive secondary display 13362, 13364.

In various instances, the processor of a robotic surgical system, such as the robotic surgical systems 13000 (FIG. 4), 13400 (FIG. 5), 13360 (FIG. 6), and/or the surgical hub 13380, 13382, for example, is programmed with pre-approved functions of the robotic surgical system. For example, if a clinician input from the interactive secondary display 13362, 13364 corresponds to a pre-approved function, the robotic surgical system allows for the interactive secondary display 13362, 13364 to control the robotic surgical system and/or does not prohibit the interactive secondary display 13362, 13364 from controlling the robotic surgical system. If a clinician input from the interactive secondary display 13362, 13364 does not correspond to a pre-approved function, the interactive secondary display 13362, 13364 is unable to command the robotic surgical system to perform the desired function. In one instances, a situational awareness module in the robotic hub 13380 and/or the surgical hub 13382 is configured to dictate and/or influence when the interactive secondary display can issue control motions to the robot surgical system.

In various instances, an interactive secondary display 13362, 13364 has control over a portion of the robotic surgical system upon making contact with the portion of the robotic surgical system. For example, when the interactive secondary display 13362, 13364 is brought into contact with the robotic tool 13374, control of the contacted robotic tool 13374 is granted to the interactive secondary display 13362, 13364. A clinician can then utilize a touch-sensitive screen and/or buttons on the interactive secondary display 13362, 13364 to input a command to control movement and/or functionality of the contacted robotic tool 13374. This control scheme allows for a clinician to reposition a robotic arm, reload a robotic tool, and/or otherwise reconfigure the robotic surgical system. In a similar manner as discussed above, the clinician 13371 positioned at the remote command console 13370 of the robotic surgical system can manually override any robot command initiated by the interactive secondary display 13362, 13364.

In one aspect, the robotic surgical system includes a processor and a memory communicatively coupled to the processor, as described herein. The memory stores instructions executable by the processor to receive a first user input from a console and to receive a second user input from a mobile wireless control module for controlling a function of a robotic surgical tool, as described herein.

In various aspects, the present disclosure provides a control circuit to receive a first user input from a console and to receive a second user input from a mobile wireless control module for controlling a function of a robotic surgical tool, as described herein. In various aspects, the present disclosure provides a non-transitory computer readable medium storing computer readable instructions which, when executed, cause a machine to receive a first user input from a console and to receive a second user input from a mobile wireless control module for controlling a function of a robotic surgical tool, as described herein.

A robotic surgical system may include multiple robotic arms that are configured to assist the clinician during a surgical procedure. Each robotic arm may be operable independently of the others. A lack of communication may exist between each of the robotic arms as they are independently operated, which may increase the risk of tissue trauma. For example, in a scenario where one robotic arm is configured to apply a force that is stronger and in a different direction than a force configured to be applied by a second robotic arm, tissue trauma can result. For example, tissue trauma and/or tearing may occur when a first robotic arm applies a strong retracting force to the tissue while a second robotic arm is configured to rigidly hold the tissue in place.

In various instances, one or more sensors are attached to each robotic arm of a robotic surgical system. The one or more sensors are configured to sense a force applied to the surrounding tissue during the operation of the robotic arm. Such forces can include, for example, a holding force, a retracting force, and/or a dragging force. The sensor from each robotic arm is configured to communicate the magnitude and direction of the detected force to a control unit of the robotic surgical system. The control unit is configured to analyze the communicated forces and set limits for maximum loads to avoid causing trauma to the tissue in a surgical site. For example, the control unit may minimize the holding force applied by a first robotic arm if the retracting or dragging force applied by a second robotic arm increases.

FIG. 4a illustrates an exemplification of a robotic arm 13120 and a tool assembly 13130 releasably coupled to the robotic arm 13120. The robotic arm 13120 can support and move the associated tool assembly 13130 along one or more mechanical degrees of freedom (e.g., all six Cartesian degrees of freedom, five or fewer Cartesian degrees of freedom, etc.).

The robotic arm 13120 can include a tool driver 13140 at a distal end of the robotic arm 13120, which can assist with controlling features associated with the tool assembly 13130. The robotic arm 13120 can also include a movable tool guide 13132 that can retract and extend relative to the tool driver 13140. A shaft of the tool assembly 13130 can extend parallel to a threaded shaft of the movable tool guide 13132 and can extend through a distal end feature 13133 (e.g., a ring) of the movable tool guide 13132 and into a patient.

In order to provide a sterile operation area while using the surgical system, a barrier can be placed between the actuating portion of the surgical system (e.g., the robotic arm 13120) and the surgical instruments (e.g., the tool assembly 13130) in the sterile surgical field. A sterile component, such as an instrument sterile adapter (ISA), can also be placed at the connecting interface between the tool assembly 13130 and the robotic arm 13120. The placement of an ISA between the tool assembly 13130 and the robotic arm 13120 can ensure a sterile coupling point for the tool assembly 13130 and the robotic arm 13120. This permits removal of tool assemblies 13130 from the robotic arm 13120 to exchange with other tool assemblies 13130 during the course of a surgery without compromising the sterile surgical field.

The tool assembly 13130 can be loaded from a top side of the tool driver 13140 with the shaft of the tool assembly 13130 being positioned in a shaft-receiving channel 13144 formed along the side of the tool driver 13140. The shaft-receiving channel 13144 allows the shaft, which extends along a central axis of the tool assembly 13130, to extend along a central axis of the tool driver 13140 when the tool assembly 13130 is coupled to the tool driver 13140. In other exemplifications, the shaft can extend through on opening in the tool driver 13140, or the two components can mate in various other configurations.

As discussed above, the robotic surgical system can include one or more robotic arms with each robotic arm having a tool assembly coupled thereto. Each tool assembly can include an end effector that has one or more of a variety of features, such as one or more tools for assisting with performing a surgical procedure. For example, the end effector can include a cutting or boring tool that can be used to perforate or cut through tissue (e.g., create an incision).

Furthermore, some end effectors include one or more sensors that can sense a variety of characteristics associated with either the end effector or the tissue. Each robotic arm and end effector can be controlled by a control system to assist with creating a desired cut or bore and prevent against undesired cutting of tissue. As an alternative to (or in addition to) controlling the robotic arm, it is understood that the control system can control either the tool itself or the tool assembly.

One or more aspects associated with the movement of the robotic arm can be controlled by the control system, such as either a direction or a velocity of movement. For example, when boring through tissue, the robotic arm can be controlled to perform jackhammer-like movements with the cutting tool. Such jackhammer movements can include the robotic arm moving up and down along an axis (e.g., an axis that is approximately perpendicular to the tissue being perforated) in a rapid motion while also advancing the cutting tool in a downward direction towards the tissue to eventually perforate the tissue with the cutting tool (e.g. an ultrasonic blade). While performing such movements in a robotic surgical procedure, not only can it be difficult to see the tissue being perforated to thereby determine a relative position of the cutting tool, but it can also be difficult to determine when the cutting tool has completed perforating the tissue. Such position of the cutting tool relative to the tissue can include the cutting tool approaching or not yet in contact with the tissue, the cutting tool drilling down or cutting into the tissue, and the cutting tool extending through or having perforated the tissue. These positions can be difficult for either a user controlling the robotic arm or the robotic surgical system to determine which can result in potential harm to the patient due to over or under-penetrating the tissue, as well as result in longer procedure times. As such, in order to reduce procedure time and surgical errors, the robotic surgical system includes a control system that communicates with at least one sensor assembly configured to sense a force applied at a distal end of the end effector or cutting tool. The control system can thereby determine and control, based on such sensed forces, one or more appropriate aspects associated with the movement of the robotic arm, such as when boring or cutting into tissue, as will be described in greater detail below.

Although a cutting tool for perforating tissue is described in detail herein, the sensor assembly of the present disclosure that is in communication with the control system can be implemented in any number of robotic surgical systems for detecting any number of a variety of tools and/or end effectors used for performing any number of a variety of procedures without departing from the scope of this disclosure. Furthermore, any number of movements can be performed by the robotic arm to perforate or cut tissue using the robotic surgical system including the sensor assembly and control system described herein and is not limited to the jackhammering or boring of tissue.

FIG. 4a and additional exemplifications are further described in U.S. patent application Ser. No. 15/237,753, entitled CONTROL OF ADVANCEMENT RATE AND APPLICATION FORCE BASED ON MEASURED FORCES, filed Aug. 16, 2016, the entire disclosure of which is incorporated by reference herein.

The entire disclosures of:
U.S. Pat. No. 9,072,535, filed May 27, 2011, entitled SURGICAL STAPLING INSTRUMENTS WITH ROTATABLE STAPLE DEPLOYMENT ARRANGEMENTS, which issued Jul. 7, 2015;
U.S. Pat. No. 9,072,536, filed Jun. 28, 2012, entitled DIFFERENTIAL LOCKING ARRANGEMENTS FOR ROTARY POWERED SURGICAL INSTRUMENTS, which issued Jul. 7, 2015;
U.S. Pat. No. 9,204,879, filed Jun. 28, 2012, entitled FLEXIBLE DRIVE MEMBER, which issued on Dec. 8, 2015;
U.S. Pat. No. 9,561,038, filed Jun. 28, 2012, entitled INTERCHANGEABLE CLIP APPLIER, which issued on Feb. 7, 2017;
U.S. Pat. No. 9,757,128, filed Sep. 5, 2014, entitled MULTIPLE SENSORS WITH ONE SENSOR AFFECTING A SECOND SENSOR'S OUTPUT OR INTERPRETATION, which issued on Sep. 12, 2017;
U.S. patent application Ser. No. 14/640,935, entitled OVERLAID MULTI SENSOR RADIO FREQUENCY (RF) ELECTRODE SYSTEM TO MEASURE TISSUE COMPRESSION, filed Mar. 6, 2015, now U.S. Patent Application Publication No. 2016/0256071;
U.S. patent application Ser. No. 15/382,238, entitled MODULAR BATTERY POWERED HANDHELD SURGICAL INSTRUMENT WITH SELECTIVE APPLICATION OF ENERGY BASED ON TISSUE CHARACTERIZATION, filed Dec. 16, 2016, now U.S. Patent Application Publication No. 2017/0202591; and
U.S. patent application Ser. No. 15/237,752, entitled CONTROL OF ADVANCEMENT RATE AND APPLICATION FORCE BASED ON MEASURED FORCES, filed Aug. 16, 2016 are hereby incorporated by reference herein in their respective entireties.

The surgical devices, systems, and methods disclosed herein can be implemented with a variety of different robotic surgical systems and surgical devices. Surgical devices include robotic surgical tools and handheld surgical instruments. The reader will readily appreciate that certain devices, systems, and methods disclosed herein are not limited to applications within a robotic surgical system. For example, certain systems, devices, and methods for communicating, detecting, and/or control a surgical device can be implemented without a robotic surgical system.

### Surgical Network

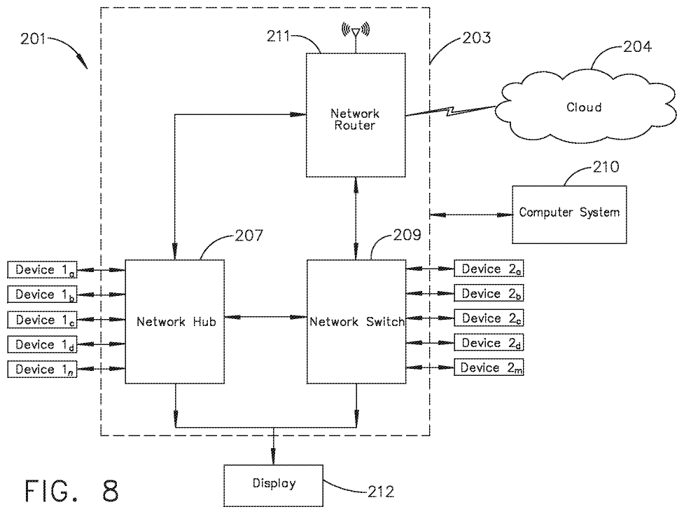

FIG. 8 illustrates a surgical data network 201 comprising a modular communication hub 203 configured to connect modular devices located in one or more operating theaters of a healthcare facility, or any room in a healthcare facility specially equipped for surgical operations, to a cloud-based system (e.g., the cloud 204 that may include a remote server 213 coupled to a storage device 205). In one aspect, the modular communication hub 203 comprises a network hub 207 and/or a network switch 209 in communication with a network router. The modular communication hub 203 also can be coupled to a local computer system 210 to provide local computer processing and data manipulation. The surgical data network 201 may be configured as passive, intelligent, or switching. A passive surgical data network serves as a conduit for the data, enabling it to go from one device (or segment) to another and to the cloud computing resources. An intelligent surgical data network includes additional features to enable the traffic passing through the surgical data network to be monitored and to configure each port in the network hub 207 or network switch 209. An intelligent surgical data network may be referred to as a manageable hub or switch. A switching hub reads the destination address of each packet and then forwards the packet to the correct port.

Modular devices 1a-1n located in the operating theater may be coupled to the modular communication hub 203. The network hub 207 and/or the network switch 209 may be coupled to a network router 211 to connect the devices 1a-1n to the cloud 204 or the local computer system 210. Data associated with the devices 1a-1n may be transferred to cloud-based computers via the router for remote data processing and manipulation. Data associated with the devices 1a-1n may also be transferred to the local computer system 210 for local data processing and manipulation. Modular devices 2a-2m located in the same operating theater also may be coupled to a network switch 209. The network switch 209 may be coupled to the network hub 207 and/or the network router 211 to connect to the devices 2a-2m to the cloud 204. Data associated with the devices 2a-2n may be transferred to the cloud 204 via the network router 211 for data processing and manipulation. Data associated with the devices 2a-2m may also be transferred to the local computer system 210 for local data processing and manipulation.

It will be appreciated that the surgical data network 201 may be expanded by interconnecting multiple network hubs 207 and/or multiple network switches 209 with multiple network routers 211. The modular communication hub 203 may be contained in a modular control tower configured to receive multiple devices 1a-1n/2a-2m. The local computer system 210 also may be contained in a modular control tower. The modular communication hub 203 is connected to a display 212 to display images obtained by some of the devices 1a-1n/2a-2m, for example during surgical procedures. In various aspects, the devices 1a-1n/2a-2m may include, for example, various modules such as an imaging module 138 coupled to an endoscope, a generator module 140 coupled to an energy-based surgical device, a smoke evacuation module 126, a suction/irrigation module 128, a communication module 130, a processor module 132, a storage array 134, a surgical device coupled to a display, and/or a non-contact sensor module, among other modular devices that may be connected to the modular communication hub 203 of the surgical data network 201.

In one aspect, the surgical data network 201 may comprise a combination of network hub(s), network switch(es), and network router(s) connecting the devices 1a-1n/2a-2m to the cloud. Any one of or all of the devices 1a-1n/2a-2m coupled to the network hub or network switch may collect data in real time and transfer the data to cloud computers for data processing and manipulation. It will be appreciated that cloud computing relies on sharing computing resources rather than having local servers or personal devices to handle software applications. The word “cloud” may be used as a metaphor for “the Internet,” although the term is not limited as such. Accordingly, the term “cloud computing” may be used herein to refer to “a type of Internet-based computing,” where different services—such as servers, storage, and applications—are delivered to the modular communication hub 203 and/or computer system 210 located in the surgical theater (e.g., a fixed, mobile, temporary, or field operating room or space) and to devices connected to the modular communication hub 203 and/or computer system 210 through the Internet. The cloud infrastructure may be maintained by a cloud service provider. In this context, the cloud service provider may be the entity that coordinates the usage and control of the devices 1a-1n/2a-2m located in one or more operating theaters. The cloud computing services can perform a large number of calculations based on the data gathered by smart surgical instruments, robots, and other computerized devices located in the operating theater. The hub hardware enables multiple devices or connections to be connected to a computer that communicates with the cloud computing resources and storage.

Applying cloud computer data processing techniques on the data collected by the devices 1a-1n/2a-2m, the surgical data network provides improved surgical outcomes, reduced costs, and improved patient satisfaction. At least some of the devices 1a-1n/2a-2m may be employed to view tissue states to assess leaks or perfusion of sealed tissue after a tissue sealing and cutting procedure. At least some of the devices 1a-1n/2a-2m may be employed to identify pathology, such as the effects of diseases, using the cloud-based computing to examine data including images of samples of body tissue for diagnostic purposes. This includes localization and margin confirmation of tissue and phenotypes. At least some of the devices 1a-1n/2a-2m may be employed to identify anatomical structures of the body using a variety of sensors integrated with imaging devices and techniques such as overlaying images captured by multiple imaging devices. The data gathered by the devices 1a-1n/2a-2m, including image data, may be transferred to the cloud 204 or the local computer system 210 or both for data processing and manipulation including image processing and manipulation. The data may be analyzed to improve surgical procedure outcomes by determining if further treatment, such as the application of endoscopic intervention, emerging technologies, a targeted radiation, targeted intervention, and precise robotics to tissue-specific sites and conditions, may be pursued. Such data analysis may further employ outcome analytics processing, and using standardized approaches may provide beneficial feedback to either confirm surgical treatments and the behavior of the surgeon or suggest modifications to surgical treatments and the behavior of the surgeon.

In one implementation, the operating theater devices 1a-1n may be connected to the modular communication hub 203 over a wired channel or a wireless channel depending on the configuration of the devices 1a-1n to a network hub. The network hub 207 may be implemented, in one aspect, as a local network broadcast device that works on the physical layer of the Open System Interconnection (OSI) model. The network hub provides connectivity to the devices 1a-1n located in the same operating theater network. The network hub 207 collects data in the form of packets and sends them to the router in half duplex mode. The network hub 207 does not store any media access control/internet protocol (MAC/IP) to transfer the device data. Only one of the devices 1a-1n can send data at a time through the network hub 207. The network hub 207 has no routing tables or intelligence regarding where to send information and broadcasts all network data across each connection and to a remote server 213 (FIG. 9) over the cloud 204. The network hub 207 can detect basic network errors such as collisions, but having all information broadcast to multiple ports can be a security risk and cause bottlenecks.

In another implementation, the operating theater devices 2a-2m may be connected to a network switch 209 over a wired channel or a wireless channel. The network switch 209 works in the data link layer of the OSI model. The network switch 209 is a multicast device for connecting the devices 2a-2m located in the same operating theater to the network. The network switch 209 sends data in the form of frames to the network router 211 and works in full duplex mode. Multiple devices 2a-2m can send data at the same time through the network switch 209. The network switch 209 stores and uses MAC addresses of the devices 2a-2m to transfer data.

The network hub 207 and/or the network switch 209 are coupled to the network router 211 for connection to the cloud 204. The network router 211 works in the network layer of the OSI model. The network router 211 creates a route for transmitting data packets received from the network hub 207 and/or network switch 211 to cloud-based computer resources for further processing and manipulation of the data collected by any one of or all the devices 1a-1n/2a-2m. The network router 211 may be employed to connect two or more different networks located in different locations, such as, for example, different operating theaters of the same healthcare facility or different networks located in different operating theaters of different healthcare facilities. The network router 211 sends data in the form of packets to the cloud 204 and works in full duplex mode. Multiple devices can send data at the same time. The network router 211 uses IP addresses to transfer data.

In one example, the network hub 207 may be implemented as a USB hub, which allows multiple USB devices to be connected to a host computer. The USB hub may expand a single USB port into several tiers so that there are more ports available to connect devices to the host system computer. The network hub 207 may include wired or wireless capabilities to receive information over a wired channel or a wireless channel. In one aspect, a wireless USB short-range, high-bandwidth wireless radio communication protocol may be employed for communication between the devices 1a-1n and devices 2a-2m located in the operating theater.

In other examples, the operating theater devices 1a-1n/2a-2m may communicate to the modular communication hub 203 via Bluetooth wireless technology standard for exchanging data over short distances (using short-wavelength UHF radio waves in the ISM band from 2.4 to 2.485 GHz) from fixed and mobile devices and building personal area networks (PANs). In other aspects, the operating theater devices 1a-1n/2a-2m may communicate to the modular communication hub 203 via a number of wireless or wired communication standards or protocols, including but not limited to Wi-Fi (IEEE 802.11 family), WiMAX (IEEE 802.16 family), IEEE 802.20, long-term evolution (LTE), and Ev-DO, HSPA+, HSDPA+, HSUPA+, EDGE, GSM, GPRS, CDMA, TDMA, DECT, and Ethernet derivatives thereof, as well as any other wireless and wired protocols that are designated as 3G, 4G, 5G, and beyond. The computing module may include a plurality of communication modules. For instance, a first communication module may be dedicated to shorter-range wireless communications such as Wi-Fi and Bluetooth, and a second communication module may be dedicated to longer-range wireless communications such as GPS, EDGE, GPRS, CDMA, WiMAX, LTE, Ev-DO, and others.

The modular communication hub 203 may serve as a central connection for one or all of the operating theater devices 1a-1n/2a-2m and handles a data type known as frames. Frames carry the data generated by the devices 1a-1n/2a-2m. When a frame is received by the modular communication hub 203, it is amplified and transmitted to the network router 211, which transfers the data to the cloud computing resources by using a number of wireless or wired communication standards or protocols, as described herein.

The modular communication hub 203 can be used as a standalone device or be connected to compatible network hubs and network switches to form a larger network. The modular communication hub 203 is generally easy to install, configure, and maintain, making it a good option for networking the operating theater devices 1a-1n/2a-2m.

FIG. 9 illustrates a computer-implemented interactive surgical system 200. The computer-implemented interactive surgical system 200 is similar in many respects to the computer-implemented interactive surgical system 100. For example, the computer-implemented interactive surgical system 200 includes one or more surgical systems 202, which are similar in many respects to the surgical systems 102. Each surgical system 202 includes at least one surgical hub 206 in communication with a cloud 204 that may include a remote server 213. In one aspect, the computer-implemented interactive surgical system 200 comprises a modular control tower 236 connected to multiple operating theater devices such as, for example, intelligent surgical instruments, robots, and other computerized devices located in the operating theater. As shown in FIG. 10, the modular control tower 236 comprises a modular communication hub 203 coupled to a computer system 210. As illustrated in the example of FIG. 9, the modular control tower 236 is coupled to an imaging module 238 that is coupled to an endoscope 239, a generator module 240 that is coupled to an energy device 241, a smoke evacuator module 226, a suction/irrigation module 228, a communication module 230, a processor module 232, a storage array 234, a smart device/instrument 235 optionally coupled to a display 237, and a non-contact sensor module 242. The operating theater devices are coupled to cloud computing resources and data storage via the modular control tower 236. A robot hub 222 also may be connected to the modular control tower 236 and to the cloud computing resources. The devices/instruments 235, visualization systems 208, among others, may be coupled to the modular control tower 236 via wired or wireless communication standards or protocols, as described herein. The modular control tower 236 may be coupled to a hub display 215 (e.g., monitor, screen) to display and overlay images received from the imaging module, device/instrument display, and/or other visualization systems 208. The hub display also may display data received from devices connected to the modular control tower in conjunction with images and overlaid images.

FIG. 10 illustrates a surgical hub 206 comprising a plurality of modules coupled to the modular control tower 236. The modular control tower 236 comprises a modular communication hub 203, e.g., a network connectivity device, and a computer system 210 to provide local processing, visualization, and imaging, for example. As shown in FIG. 10, the modular communication hub 203 may be connected in a tiered configuration to expand the number of modules (e.g., devices) that may be connected to the modular communication hub 203 and transfer data associated with the modules to the computer system 210, cloud computing resources, or both. As shown in FIG. 10, each of the network hubs/switches in the modular communication hub 203 includes three downstream ports and one upstream port. The upstream network hub/switch is connected to a processor to provide a communication connection to the cloud computing resources and a local display 217. Communication to the cloud 204 may be made either through a wired or a wireless communication channel.

The surgical hub 206 employs a non-contact sensor module 242 to measure the dimensions of the operating theater and generate a map of the surgical theater using either ultrasonic or laser-type non-contact measurement devices. An ultrasound-based non-contact sensor module scans the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, the disclosure of which is herein incorporated by reference in its entirety, in which the sensor module is configured to determine the size of the operating theater and to adjust Bluetooth-pairing distance limits. A laser-based non-contact sensor module scans the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

The computer system 210 comprises a processor 244 and a network interface 245. The processor 244 is coupled to a communication module 247, storage 248, memory 249, non-volatile memory 250, and input/output interface 251 via a system bus. The system bus can be any of several types of bus structure(s) including the memory bus or memory controller, a peripheral bus or external bus, and/or a local bus using any variety of available bus architectures including, but not limited to, 9-bit bus, Industrial Standard Architecture (ISA), Micro-Charmel Architecture (MSA), Extended ISA (EISA), Intelligent Drive Electronics (IDE), VESA Local Bus (VLB), Peripheral Component Interconnect (PCI), USB, Advanced Graphics Port (AGP), Personal Computer Memory Card International Association bus (PCMCIA), Small Computer Systems Interface (SCSI), or any other proprietary bus.

The processor 244 may be any single-core or multicore processor such as those known under the trade name ARM Cortex by Texas Instruments. In one aspect, the processor may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle serial random access memory (SRAM), an internal read-only memory (ROM) loaded with StellarisWare® software, a 2 KB electrically erasable programmable read-only memory (EEPROM), and/or one or more pulse width modulation (PWM) modules, one or more quadrature encoder inputs (QEI) analogs, one or more 12-bit analog-to-digital converters (ADCs) with 12 analog input channels, details of which are available for the product datasheet.

In one aspect, the processor 244 may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The system memory includes volatile memory and non-volatile memory. The basic input/output system (BIOS), containing the basic routines to transfer information between elements within the computer system, such as during start-up, is stored in non-volatile memory. For example, the non-volatile memory can include ROM, programmable ROM (PROM), electrically programmable ROM (EPROM), EEPROM, or flash memory. Volatile memory includes random-access memory (RAM), which acts as external cache memory. Moreover, RAM is available in many forms such as SRAM, dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM).

The computer system 210 also includes removable/non-removable, volatile/non-volatile computer storage media, such as for example disk storage. The disk storage includes, but is not limited to, devices like a magnetic disk drive, floppy disk drive, tape drive, Jaz drive, Zip drive, LS-60 drive, flash memory card, or memory stick. In addition, the disk storage can include storage media separately or in combination with other storage media including, but not limited to, an optical disc drive such as a compact disc ROM device (CD-ROM), compact disc recordable drive (CD-R Drive), compact disc rewritable drive (CD-RW Drive), or a digital versatile disc ROM drive (DVD-ROM). To facilitate the connection of the disk storage devices to the system bus, a removable or non-removable interface may be employed.

It is to be appreciated that the computer system 210 includes software that acts as an intermediary between users and the basic computer resources described in a suitable operating environment. Such software includes an operating system. The operating system, which can be stored on the disk storage, acts to control and allocate resources of the computer system. System applications take advantage of the management of resources by the operating system through program modules and program data stored either in the system memory or on the disk storage. It is to be appreciated that various components described herein can be implemented with various operating systems or combinations of operating systems.

A user enters commands or information into the computer system 210 through input device(s) coupled to the I/O interface 251. The input devices include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processor through the system bus via interface port(s). The interface port(s) include, for example, a serial port, a parallel port, a game port, and a USB. The output device(s) use some of the same types of ports as input device(s). Thus, for example, a USB port may be used to provide input to the computer system and to output information from the computer system to an output device. An output adapter is provided to illustrate that there are some output devices like monitors, displays, speakers, and printers, among other output devices that require special adapters. The output adapters include, by way of illustration and not limitation, video and sound cards that provide a means of connection between the output device and the system bus. It should be noted that other devices and/or systems of devices, such as remote computer(s), provide both input and output capabilities.

The computer system 210 can operate in a networked environment using logical connections to one or more remote computers, such as cloud computer(s), or local computers. The remote cloud computer(s) can be a personal computer, server, router, network PC, workstation, microprocessor-based appliance, peer device, or other common network node, and the like, and typically includes many or all of the elements described relative to the computer system. For purposes of brevity, only a memory storage device is illustrated with the remote computer(s). The remote computer(s) is logically connected to the computer system through a network interface and then physically connected via a communication connection. The network interface encompasses communication networks such as local area networks (LANs) and wide area networks (WANs). LAN technologies include Fiber Distributed Data Interface (FDDI), Copper Distributed Data Interface (CDDI), Ethernet/IEEE 802.3, Token Ring/IEEE 802.5 and the like. WAN technologies include, but are not limited to, point-to-point links, circuit-switching networks like Integrated Services Digital Networks (ISDN) and variations thereon, packet-switching networks, and Digital Subscriber Lines (DSL).

In various aspects, the computer system 210 of FIG. 10, the imaging module 238 and/or visualization system 208, and/or the processor module 232 of FIGS. 9-10, may comprise an image processor, image processing engine, media processor, or any specialized digital signal processor (DSP) used for the processing of digital images. The image processor may employ parallel computing with single instruction, multiple data (SIMD) or multiple instruction, multiple data (MIMD) technologies to increase speed and efficiency. The digital image processing engine can perform a range of tasks. The image processor may be a system on a chip with multicore processor architecture.

The communication connection(s) refers to the hardware/software employed to connect the network interface to the bus. While the communication connection is shown for illustrative clarity inside the computer system, it can also be external to the computer system 210. The hardware/software necessary for connection to the network interface includes, for illustrative purposes only, internal and external technologies such as modems, including regular telephone-grade modems, cable modems, and DSL modems, ISDN adapters, and Ethernet cards.

FIG. 11 illustrates a functional block diagram of one aspect of a USB network hub 300 device, according to one aspect of the present disclosure. In the illustrated aspect, the USB network hub device 300 employs a TUSB2036 integrated circuit hub by Texas Instruments. The USB network hub 300 is a CMOS device that provides an upstream USB transceiver port 302 and up to three downstream USB transceiver ports 304, 306, 308 in compliance with the USB 2.0 specification. The upstream USB transceiver port 302 is a differential root data port comprising a differential data minus (DM0) input paired with a differential data plus (DP0) input. The three downstream USB transceiver ports 304, 306, 308 are differential data ports where each port includes differential data plus (DP1-DP3) outputs paired with differential data minus (DM1-DM3) outputs.

The USB network hub 300 device is implemented with a digital state machine instead of a microcontroller, and no firmware programming is required. Fully compliant USB transceivers are integrated into the circuit for the upstream USB transceiver port 302 and all downstream USB transceiver ports 304, 306, 308. The downstream USB transceiver ports 304, 306, 308 support both full-speed and low-speed devices by automatically setting the slew rate according to the speed of the device attached to the ports. The USB network hub 300 device may be configured either in bus-powered or self-powered mode and includes a hub power logic 312 to manage power.

The USB network hub 300 device includes a serial interface engine 310 (SIE). The SIE 310 is the front end of the USB network hub 300 hardware and handles most of the protocol described in chapter 8 of the USB specification. The SIE 310 typically comprehends signaling up to the transaction level. The functions that it handles could include: packet recognition, transaction sequencing, SOP, EOP, RESET, and RESUME signal detection/generation, clock/data separation, non-return-to-zero invert (NRZI) data encoding/decoding and bit-stuffing, CRC generation and checking (token and data), packet ID (PID) generation and checking/decoding, and/or serial-parallel/parallel-serial conversion. The 310 receives a clock input 314 and is coupled to a suspend/resume logic and frame timer 316 circuit and a hub repeater circuit 318 to control communication between the upstream USB transceiver port 302 and the downstream USB transceiver ports 304, 306, 308 through port logic circuits 320, 322, 324. The SIE 310 is coupled to a command decoder 326 via interface logic to control commands from a serial EEPROM via a serial EEPROM interface 330.

In various aspects, the USB network hub 300 can connect 127 functions configured in up to six logical layers (tiers) to a single computer. Further, the USB network hub 300 can connect to all peripherals using a standardized four-wire cable that provides both communication and power distribution. The power configurations are bus-powered and self-powered modes. The USB network hub 300 may be configured to support four modes of power management: a bus-powered hub, with either individual-port power management or ganged-port power management, and the self-powered hub, with either individual-port power management or ganged-port power management. In one aspect, using a USB cable, the USB network hub 300, the upstream USB transceiver port 302 is plugged into a USB host controller, and the downstream USB transceiver ports 304, 306, 308 are exposed for connecting USB compatible devices, and so forth.

### Surgical Instrument Hardware

FIG. 12 illustrates a logic diagram of a control system 470 of a surgical instrument or tool in accordance with one or more aspects of the present disclosure. The system 470 comprises a control circuit. The control circuit includes a microcontroller 461 comprising a processor 462 and a memory 468. One or more of sensors 472, 474, 476, for example, provide real-time feedback to the processor 462. A motor 482, driven by a motor driver 492, operably couples a longitudinally movable displacement member to drive the I-beam knife element. A tracking system 480 is configured to determine the position of the longitudinally movable displacement member. The position information is provided to the processor 462, which can be programmed or configured to determine the position of the longitudinally movable drive member as well as the position of a firing member, firing bar, and I-beam knife element. Additional motors may be provided at the tool driver interface to control !-beam firing, closure tube travel, shaft rotation, and articulation. A display 473 displays a variety of operating conditions of the instruments and may include touch screen functionality for data input. Information displayed on the display 473 may be overlaid with images acquired via endoscopic imaging modules.

In one aspect, the microcontroller 461 may be any single-core or multicore processor such as those known under the trade name ARM Cortex by Texas Instruments. In one aspect, the main microcontroller 461 may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

In one aspect, the microcontroller 461 may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The microcontroller 461 may be programmed to perform various functions such as precise control over the speed and position of the knife and articulation systems. In one aspect, the microcontroller 461 includes a processor 462 and a memory 468. The electric motor 482 may be a brushed direct current (DC) motor with a gearbox and mechanical links to an articulation or knife system. In one aspect, a motor driver 492 may be an A3941 available from Allegro Microsystems, Inc. Other motor drivers may be readily substituted for use in the tracking system 480 comprising an absolute positioning system. A detailed description of an absolute positioning system is described in U.S. Patent Application Publication No. 2017/0296213, titled SYSTEMS AND METHODS FOR CONTROLLING A SURGICAL STAPLING AND CUTTING INSTRUMENT, which published on Oct. 19, 2017, which is herein incorporated by reference in its entirety.

The microcontroller 461 may be programmed to provide precise control over the speed and position of displacement members and articulation systems. The microcontroller 461 may be configured to compute a response in the software of the microcontroller 461. The computed response is compared to a measured response of the actual system to obtain an “observed” response, which is used for actual feedback decisions. The observed response is a favorable, tuned value that balances the smooth, continuous nature of the simulated response with the measured response, which can detect outside influences on the system.

In one aspect, the motor 482 may be controlled by the motor driver 492 and can be employed by the firing system of the surgical instrument or tool. In various forms, the motor 482 may be a brushed DC driving motor having a maximum rotational speed of approximately 25,000 RPM. In other arrangements, the motor 482 may include a brushless motor, a cordless motor, a synchronous motor, a stepper motor, or any other suitable electric motor. The motor driver 492 may comprise an H-bridge driver comprising field-effect transistors (FETs), for example. The motor 482 can be powered by a power assembly releasably mounted to the handle assembly or tool housing for supplying control power to the surgical instrument or tool. The power assembly may comprise a battery which may include a number of battery cells connected in series that can be used as the power source to power the surgical instrument or tool. In certain circumstances, the battery cells of the power assembly may be replaceable and/or rechargeable. In at least one example, the battery cells can be lithium-ion batteries which can be couplable to and separable from the power assembly.

The motor driver 492 may be an A3941 available from Allegro Microsystems, Inc. The A3941 492 is a full-bridge controller for use with external N-channel power metal-oxide semiconductor field-effect transistors (MOSFETs) specifically designed for inductive loads, such as brush DC motors. The driver 492 comprises a unique charge pump regulator that provides full (>10 V) gate drive for battery voltages down to 7 V and allows the A3941 to operate with a reduced gate drive, down to 5.5 V. A bootstrap capacitor may be employed to provide the above battery supply voltage required for N-channel MOSFETs. An internal charge pump for the high-side drive allows DC (100% duty cycle) operation. The full bridge can be driven in fast or slow decay modes using diode or synchronous rectification. In the slow decay mode, current recirculation can be through the high-side or the lowside FETs. The power FETs are protected from shoot-through by resistor-adjustable dead time. Integrated diagnostics provide indications of undervoltage, overtemperature, and power bridge faults and can be configured to protect the power MOSFETs under most short circuit conditions. Other motor drivers may be readily substituted for use in the tracking system 480 comprising an absolute positioning system.

The tracking system 480 comprises a controlled motor drive circuit arrangement comprising a position sensor 472 according to one aspect of this disclosure. The position sensor 472 for an absolute positioning system provides a unique position signal corresponding to the location of a displacement member. In one aspect, the displacement member represents a longitudinally movable drive member comprising a rack of drive teeth for meshing engagement with a corresponding drive gear of a gear reducer assembly. In other aspects, the displacement member represents the firing member, which could be adapted and configured to include a rack of drive teeth. In yet another aspect, the displacement member represents a firing bar or the I-beam, each of which can be adapted and configured to include a rack of drive teeth. Accordingly, as used herein, the term displacement member is used generically to refer to any movable member of the surgical instrument or tool such as the drive member, the firing member, the firing bar, the I-beam, or any element that can be displaced. In one aspect, the longitudinally movable drive member is coupled to the firing member, the firing bar, and the I-beam. Accordingly, the absolute positioning system can, in effect, track the linear displacement of the I-beam by tracking the linear displacement of the longitudinally movable drive member. In various other aspects, the displacement member may be coupled to any position sensor 472 suitable for measuring linear displacement. Thus, the longitudinally movable drive member, the firing member, the firing bar, or the I-beam, or combinations thereof, may be coupled to any suitable linear displacement sensor. Linear displacement sensors may include contact or non-contact displacement sensors. Linear displacement sensors may comprise linear variable differential transformers (LVDT), differential variable reluctance transducers (DVRT), a slide potentiometer, a magnetic sensing system comprising a movable magnet and a series of linearly arranged Hall effect sensors, a magnetic sensing system comprising a fixed magnet and a series of movable, linearly arranged Hall effect sensors, an optical sensing system comprising a movable light source and a series of linearly arranged photo diodes or photo detectors, an optical sensing system comprising a fixed light source and a series of movable linearly, arranged photo diodes or photo detectors, or any combination thereof.

The electric motor 482 can include a rotatable shaft that operably interfaces with a gear assembly that is mounted in meshing engagement with a set, or rack, of drive teeth on the displacement member. A sensor element may be operably coupled to a gear assembly such that a single revolution of the position sensor 472 element corresponds to some linear longitudinal translation of the displacement member. An arrangement of gearing and sensors can be connected to the linear actuator, via a rack and pinion arrangement, or a rotary actuator, via a spur gear or other connection. A power source supplies power to the absolute positioning system and an output indicator may display the output of the absolute positioning system. The displacement member represents the longitudinally movable drive member comprising a rack of drive teeth formed thereon for meshing engagement with a corresponding drive gear of the gear reducer assembly. The displacement member represents the longitudinally movable firing member, firing bar, I-beam, or combinations thereof.

A single revolution of the sensor element associated with the position sensor 472 is equivalent to a longitudinal linear displacement d1 of the of the displacement member, where d1 is the longitudinal linear distance that the displacement member moves from point “a” to point “b” after a single revolution of the sensor element coupled to the displacement member. The sensor arrangement may be connected via a gear reduction that results in the position sensor 472 completing one or more revolutions for the full stroke of the displacement member. The position sensor 472 may complete multiple revolutions for the full stroke of the displacement member.

A series of switches, where n is an integer greater than one, may be employed alone or in combination with a gear reduction to provide a unique position signal for more than one revolution of the position sensor 472. The state of the switches are fed back to the microcontroller 461 that applies logic to determine a unique position signal corresponding to the longitudinal linear displacement d1+d2+ . . . dn of the displacement member. The output of the position sensor 472 is provided to the microcontroller 461. The position sensor 472 of the sensor arrangement may comprise a magnetic sensor, an analog rotary sensor like a potentiometer, or an array of analog Hall-effect elements, which output a unique combination of position signals or values.

The position sensor 472 may comprise any number of magnetic sensing elements, such as, for example, magnetic sensors classified according to whether they measure the total magnetic field or the vector components of the magnetic field. The techniques used to produce both types of magnetic sensors encompass many aspects of physics and electronics. The technologies used for magnetic field sensing include search coil, fluxgate, optically pumped, nuclear precession, SQUID, Hall-effect, anisotropic magnetoresistance, giant magnetoresistance, magnetic tunnel junctions, giant magnetoimpedance, magnetostrictive/piezoelectric composites, magnetodiode, magnetotransistor, fiber-optic, magneto-optic, and microelectromechanical systems-based magnetic sensors, among others.

In one aspect, the position sensor 472 for the tracking system 480 comprising an absolute positioning system comprises a magnetic rotary absolute positioning system. The position sensor 472 may be implemented as an AS5055EQFT single-chip magnetic rotary position sensor available from Austria Microsystems, AG. The position sensor 472 is interfaced with the microcontroller 461 to provide an absolute positioning system. The position sensor 472 is a low-voltage and low-power component and includes four Hall-effect elements in an area of the position sensor 472 that is located above a magnet. A high-resolution ADC and a smart power management controller are also provided on the chip. A coordinate rotation digital computer (CORDIC) processor, also known as the digit-by-digit method and Volder's algorithm, is provided to implement a simple and efficient algorithm to calculate hyperbolic and trigonometric functions that require only addition, subtraction, bitshift, and table lookup operations. The angle position, alarm bits, and magnetic field information are transmitted over a standard serial communication interface, such as a serial peripheral interface (SPI) interface, to the microcontroller 461. The position sensor 472 provides 12 or 14 bits of resolution. The position sensor 472 may be an AS5055 chip provided in a small QFN 16-pin 4×4×0.85 mm package.

The tracking system 480 comprising an absolute positioning system may comprise and/or be programmed to implement a feedback controller, such as a PID, state feedback, and adaptive controller. A power source converts the signal from the feedback controller into a physical input to the system: in this case the voltage. Other examples include a PWM of the voltage, current, and force. Other sensor(s) may be provided to measure physical parameters of the physical system in addition to the position measured by the position sensor 472. In some aspects, the other sensor(s) can include sensor arrangements such as those described in U.S. Pat. No. 9,345,481, titled STAPLE CARTRIDGE TISSUE THICKNESS SENSOR SYSTEM, which issued on May 24, 2016, which is herein incorporated by reference in its entirety; U.S. Patent Application Publication No. 2014/0263552, titled STAPLE CARTRIDGE TISSUE THICKNESS SENSOR SYSTEM, which published on Sep. 18, 2014, which is herein incorporated by reference in its entirety; and U.S. patent application Ser. No. 15/628,175, titled TECHNIQUES FOR ADAPTIVE CONTROL OF MOTOR VELOCITY OF A SURGICAL STAPLING AND CUTTING INSTRUMENT, filed Jun. 20, 2017, which is herein incorporated by reference in its entirety. In a digital signal processing system, an absolute positioning system is coupled to a digital data acquisition system where the output of the absolute positioning system will have a finite resolution and sampling frequency. The absolute positioning system may comprise a compare-and-combine circuit to combine a computed response with a measured response using algorithms, such as a weighted average and a theoretical control loop, that drive the computed response towards the measured response. The computed response of the physical system takes into account properties like mass, inertial, viscous friction, inductance resistance, etc., to predict what the states and outputs of the physical system will be by knowing the input.

The absolute positioning system provides an absolute position of the displacement member upon power-up of the instrument, without retracting or advancing the displacement member to a reset (zero or home) position as may be required with conventional rotary encoders that merely count the number of steps forwards or backwards that the motor 482 has taken to infer the position of a device actuator, drive bar, knife, or the like.

A sensor 474, such as, for example, a strain gauge or a micro-strain gauge, is configured to measure one or more parameters of the end effector, such as, for example, the amplitude of the strain exerted on the anvil during a clamping operation, which can be indicative of the closure forces applied to the anvil. The measured strain is converted to a digital signal and provided to the processor 462. Alternatively, or in addition to the sensor 474, a sensor 476, such as, for example, a load sensor, can measure the closure force applied by the closure drive system to the anvil. The sensor 476, such as, for example, a load sensor, can measure the firing force applied to an I-beam in a firing stroke of the surgical instrument or tool. The I-beam is configured to engage a wedge sled, which is configured to upwardly cam staple drivers to force out staples into deforming contact with an anvil. The I-beam also includes a sharpened cutting edge that can be used to sever tissue as the I-beam is advanced distally by the firing bar. Alternatively, a current sensor 478 can be employed to measure the current drawn by the motor 482. The force required to advance the firing member can correspond to the current drawn by the motor 482, for example. The measured force is converted to a digital signal and provided to the processor 462.

In one form, the strain gauge sensor 474 can be used to measure the force applied to the tissue by the end effector. A strain gauge can be coupled to the end effector to measure the force on the tissue being treated by the end effector. A system for measuring forces applied to the tissue grasped by the end effector comprises a strain gauge sensor 474, such as, for example, a micro-strain gauge, that is configured to measure one or more parameters of the end effector, for example. In one aspect, the strain gauge sensor 474 can measure the amplitude or magnitude of the strain exerted on a jaw member of an end effector during a clamping operation, which can be indicative of the tissue compression. The measured strain is converted to a digital signal and provided to a processor 462 of the microcontroller 461. A load sensor 476 can measure the force used to operate the knife element, for example, to cut the tissue captured between the anvil and the staple cartridge. A magnetic field sensor can be employed to measure the thickness of the captured tissue. The measurement of the magnetic field sensor also may be converted to a digital signal and provided to the processor 462.

The measurements of the tissue compression, the tissue thickness, and/or the force required to close the end effector on the tissue, as respectively measured by the sensors 474, 476, can be used by the microcontroller 461 to characterize the selected position of the firing member and/or the corresponding value of the speed of the firing member. In one instance, a memory 468 may store a technique, an equation, and/or a lookup table which can be employed by the microcontroller 461 in the assessment.

The control system 470 of the surgical instrument or tool also may comprise wired or wireless communication circuits to communicate with the modular communication hub as shown in FIGS. 8-11.

FIG. 13 illustrates a control circuit 500 configured to control aspects of the surgical instrument or tool according to one aspect of this disclosure. The control circuit 500 can be configured to implement various processes described herein. The control circuit 500 may comprise a microcontroller comprising one or more processors 502 (e.g., microprocessor, microcontroller) coupled to at least one memory circuit 504. The memory circuit 504 stores machine-executable instructions that, when executed by the processor 502, cause the processor 502 to execute machine instructions to implement various processes described herein. The processor 502 may be any one of a number of single-core or multicore processors known in the art. The memory circuit 504 may comprise volatile and non-volatile storage media. The processor 502 may include an instruction processing unit 506 and an arithmetic unit 508. The instruction processing unit may be configured to receive instructions from the memory circuit 504 of this disclosure.

FIG. 14 illustrates a combinational logic circuit 510 configured to control aspects of the surgical instrument or tool according to one aspect of this disclosure. The combinational logic circuit 510 can be configured to implement various processes described herein. The combinational logic circuit 510 may comprise a finite state machine comprising a combinational logic 512 configured to receive data associated with the surgical instrument or tool at an input 514, process the data by the combinational logic 512, and provide an output 516.

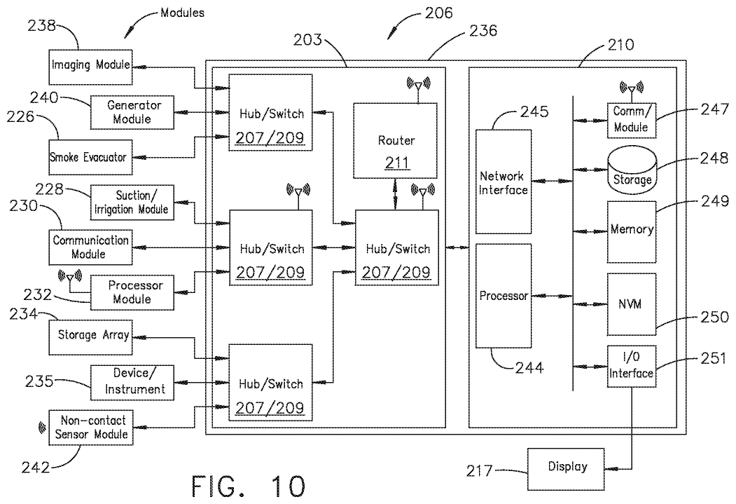

FIG. 15 illustrates a sequential logic circuit 520 configured to control aspects of the surgical instrument or tool according to one aspect of this disclosure. The sequential logic circuit 520 or the combinational logic 522 can be configured to implement various processes described herein. The sequential logic circuit 520 may comprise a finite state machine. The sequential logic circuit 520 may comprise a combinational logic 522, at least one memory circuit 524, and a clock 529, for example. The at least one memory circuit 524 can store a current state of the finite state machine. In certain instances, the sequential logic circuit 520 may be synchronous or asynchronous. The combinational logic 522 is configured to receive data associated with the surgical instrument or tool from an input 526, process the data by the combinational logic 522, and provide an output 528. In other aspects, the circuit may comprise a combination of a processor (e.g., processor 502, FIG. 13) and a finite state machine to implement various processes herein. In other aspects, the finite state machine may comprise a combination of a combinational logic circuit (e.g., combinational logic circuit 510, FIG. 14) and the sequential logic circuit 520.

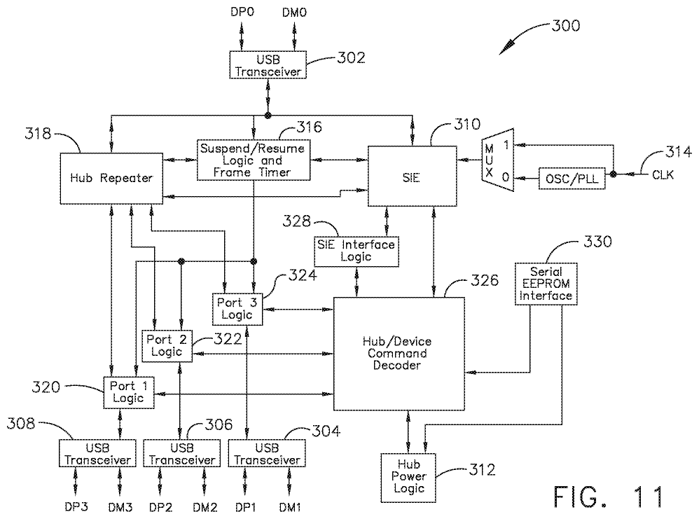

FIG. 16 illustrates a surgical instrument or tool comprising a plurality of motors which can be activated to perform various functions. In certain instances, a first motor can be activated to perform a first function, a second motor can be activated to perform a second function, a third motor can be activated to perform a third function, a fourth motor can be activated to perform a fourth function, and so on. In certain instances, the plurality of motors of robotic surgical instrument 600 can be individually activated to cause firing, closure, and/or articulation motions in the end effector. The firing, closure, and/or articulation motions can be transmitted to the end effector through a shaft assembly, for example.

In certain instances, the surgical instrument system or tool may include a firing motor 602. The firing motor 602 may be operably coupled to a firing motor drive assembly 604 which can be configured to transmit firing motions, generated by the motor 602 to the end effector, in particular to displace the I-beam element. In certain instances, the firing motions generated by the motor 602 may cause the staples to be deployed from the staple cartridge into tissue captured by the end effector and/or the cutting edge of the I-beam element to be advanced to cut the captured tissue, for example. The I-beam element may be retracted by reversing the direction of the motor 602.

In certain instances, the surgical instrument or tool may include a closure motor 603. The closure motor 603 may be operably coupled to a closure motor drive assembly 605 which can be configured to transmit closure motions, generated by the motor 603 to the end effector, in particular to displace a closure tube to close the anvil and compress tissue between the anvil and the staple cartridge. The closure motions may cause the end effector to transition from an open configuration to an approximated configuration to capture tissue, for example. The end effector may be transitioned to an open position by reversing the direction of the motor 603.

In certain instances, the surgical instrument or tool may include one or more articulation motors 606a, 606b, for example. The motors 606a, 606b may be operably coupled to respective articulation motor drive assemblies 608a, 608b, which can be configured to transmit articulation motions generated by the motors 606a, 606b to the end effector. In certain instances, the articulation motions may cause the end effector to articulate relative to the shaft, for example.

As described above, the surgical instrument or tool may include a plurality of motors which may be configured to perform various independent functions. In certain instances, the plurality of motors of the surgical instrument or tool can be individually or separately activated to perform one or more functions while the other motors remain inactive. For example, the articulation motors 606a, 606b can be activated to cause the end effector to be articulated while the firing motor 602 remains inactive. Alternatively, the firing motor 602 can be activated to fire the plurality of staples, and/or to advance the cutting edge, while the articulation motor 606 remains inactive. Furthermore the closure motor 603 may be activated simultaneously with the firing motor 602 to cause the closure tube and the I-beam element to advance distally as described in more detail hereinbelow.

In certain instances, the surgical instrument or tool may include a common control module 610 which can be employed with a plurality of motors of the surgical instrument or tool. In certain instances, the common control module 610 may accommodate one of the plurality of motors at a time. For example, the common control module 610 can be couplable to and separable from the plurality of motors of the robotic surgical instrument individually. In certain instances, a plurality of the motors of the surgical instrument or tool may share one or more common control modules such as the common control module 610. In certain instances, a plurality of motors of the surgical instrument or tool can be individually and selectively engaged with the common control module 610. In certain instances, the common control module 610 can be selectively switched from interfacing with one of a plurality of motors of the surgical instrument or tool to interfacing with another one of the plurality of motors of the surgical instrument or tool.

In at least one example, the common control module 610 can be selectively switched between operable engagement with the articulation motors 606a, 606b and operable engagement with either the firing motor 602 or the closure motor 603. In at least one example, as illustrated in FIG. 16, a switch 614 can be moved or transitioned between a plurality of positions and/or states. In a first position 616, the switch 614 may electrically couple the common control module 610 to the firing motor 602; in a second position 617, the switch 614 may electrically couple the common control module 610 to the closure motor 603; in a third position 618a, the switch 614 may electrically couple the common control module 610 to the first articulation motor 606a; and in a fourth position 618b, the switch 614 may electrically couple the common control module 610 to the second articulation motor 606b, for example. In certain instances, separate common control modules 610 can be electrically coupled to the firing motor 602, the closure motor 603, and the articulations motor 606a, 606b at the same time. In certain instances, the switch 614 may be a mechanical switch, an electromechanical switch, a solid-state switch, or any suitable switching mechanism.

Each of the motors 602, 603, 606a, 606b may comprise a torque sensor to measure the output torque on the shaft of the motor. The force on an end effector may be sensed in any conventional manner, such as by force sensors on the outer sides of the jaws or by a torque sensor for the motor actuating the jaws.

In various instances, as illustrated in FIG. 16, the common control module 610 may comprise a motor driver 626 which may comprise one or more H-Bridge FETs. The motor driver 626 may modulate the power transmitted from a power source 628 to a motor coupled to the common control module 610 based on input from a microcontroller 620 (the “controller”), for example. In certain instances, the microcontroller 620 can be employed to determine the current drawn by the motor, for example, while the motor is coupled to the common control module 610, as described above.

In certain instances, the microcontroller 620 may include a microprocessor 622 (the “processor”) and one or more non-transitory computer-readable mediums or memory units 624 (the “memory”). In certain instances, the memory 624 may store various program instructions, which when executed may cause the processor 622 to perform a plurality of functions and/or calculations described herein. In certain instances, one or more of the memory units 624 may be coupled to the processor 622, for example.

In certain instances, the power source 628 can be employed to supply power to the microcontroller 620, for example. In certain instances, the power source 628 may comprise a battery (or “battery pack” or “power pack”), such as a lithium-ion battery, for example. In certain instances, the battery pack may be configured to be releasably mounted to a handle for supplying power to the surgical instrument 600. A number of battery cells connected in series may be used as the power source 628. In certain instances, the power source 628 may be replaceable and/or rechargeable, for example.

In various instances, the processor 622 may control the motor driver 626 to control the position, direction of rotation, and/or velocity of a motor that is coupled to the common control module 610. In certain instances, the processor 622 can signal the motor driver 626 to stop and/or disable a motor that is coupled to the common control module 610. It should be understood that the term “processor” as used herein includes any suitable microprocessor, microcontroller, or other basic computing device that incorporates the functions of a computer's central processing unit (CPU) on an integrated circuit or, at most, a few integrated circuits. The processor is a multipurpose, programmable device that accepts digital data as input, processes it according to instructions stored in its memory, and provides results as output. It is an example of sequential digital logic, as it has internal memory. Processors operate on numbers and symbols represented in the binary numeral system.

In one instance, the processor 622 may be any single-core or multicore processor such as those known under the trade name ARM Cortex by Texas Instruments. In certain instances, the microcontroller 620 may be an LM 4F230H5QR, available from Texas Instruments, for example. In at least one example, the Texas Instruments LM4F230H5QR is an ARM Cortex-M4F Processor Core comprising an on-chip memory of 256 KB single-cycle flash memory, or other non-volatile memory, up to 40 MHz, a prefetch buffer to improve performance above 40 MHz, a 32 KB single-cycle SRAM, an internal ROM loaded with StellarisWare® software, a 2 KB EEPROM, one or more PWM modules, one or more QEI analogs, one or more 12-bit ADCs with 12 analog input channels, among other features that are readily available for the product datasheet. Other microcontrollers may be readily substituted for use with the module 4410. Accordingly, the present disclosure should not be limited in this context.

In certain instances, the memory 624 may include program instructions for controlling each of the motors of the surgical instrument 600 that are couplable to the common control module 610. For example, the memory 624 may include program instructions for controlling the firing motor 602, the closure motor 603, and the articulation motors 606a, 606b. Such program instructions may cause the processor 622 to control the firing, closure, and articulation functions in accordance with inputs from algorithms or control programs of the surgical instrument or tool.

In certain instances, one or more mechanisms and/or sensors such as, for example, sensors 630 can be employed to alert the processor 622 to the program instructions that should be used in a particular setting. For example, the sensors 630 may alert the processor 622 to use the program instructions associated with firing, closing, and articulating the end effector. In certain instances, the sensors 630 may comprise position sensors which can be employed to sense the position of the switch 614, for example. Accordingly, the processor 622 may use the program instructions associated with firing the I-beam of the end effector upon detecting, through the sensors 630 for example, that the switch 614 is in the first position 616; the processor 622 may use the program instructions associated with closing the anvil upon detecting, through the sensors 630 for example, that the switch 614 is in the second position 617; and the processor 622 may use the program instructions associated with articulating the end effector upon detecting, through the sensors 630 for example, that the switch 614 is in the third or fourth position 618a, 618b.

FIG. 17 is a schematic diagram of a robotic surgical instrument 700 configured to operate a surgical tool described herein according to one aspect of this disclosure. The robotic surgical instrument 700 may be programmed or configured to control distal/proximal translation of a displacement member, distal/proximal displacement of a closure tube, shaft rotation, and articulation, either with single or multiple articulation drive links. In one aspect, the surgical instrument 700 may be programmed or configured to individually control a firing member, a closure member, a shaft member, and/or one or more articulation members. The surgical instrument 700 comprises a control circuit 710 configured to control motor-driven firing members, closure members, shaft members, and/or one or more articulation members.

In one aspect, the robotic surgical instrument 700 comprises a control circuit 710 configured to control an anvil 716 and an I-beam 714 (including a sharp cutting edge) portion of an end effector 702, a removable staple cartridge 718, a shaft 740, and one or more articulation members 742a, 742b via a plurality of motors 704a-704e. A position sensor 734 may be configured to provide position feedback of the I-beam 714 to the control circuit 710. Other sensors 738 may be configured to provide feedback to the control circuit 710. A timer/counter 731 provides timing and counting information to the control circuit 710. An energy source 712 may be provided to operate the motors 704a-704e, and a current sensor 736 provides motor current feedback to the control circuit 710. The motors 704a-704e can be operated individually by the control circuit 710 in an open-loop or closed-loop feedback control.

In one aspect, the control circuit 710 may comprise one or more microcontrollers, microprocessors, or other suitable processors for executing instructions that cause the processor or processors to perform one or more tasks. In one aspect, a timer/counter 731 provides an output signal, such as the elapsed time or a digital count, to the control circuit 710 to correlate the position of the I-beam 714 as determined by the position sensor 734 with the output of the timer/counter 731 such that the control circuit 710 can determine the position of the I-beam 714 at a specific time (t) relative to a starting position or the time (t) when the I-beam 714 is at a specific position relative to a starting position. The timer/counter 731 may be configured to measure elapsed time, count external events, or time external events.

In one aspect, the control circuit 710 may be programmed to control functions of the end effector 702 based on one or more tissue conditions. The control circuit 710 may be programmed to sense tissue conditions, such as thickness, either directly or indirectly, as described herein. The control circuit 710 may be programmed to select a firing control program or closure control program based on tissue conditions. A firing control program may describe the distal motion of the displacement member. Different firing control programs may be selected to better treat different tissue conditions. For example, when thicker tissue is present, the control circuit 710 may be programmed to translate the displacement member at a lower velocity and/or with lower power. When thinner tissue is present, the control circuit 710 may be programmed to translate the displacement member at a higher velocity and/or with higher power. A closure control program may control the closure force applied to the tissue by the anvil 716. Other control programs control the rotation of the shaft 740 and the articulation members 742a, 742b.

In one aspect, the control circuit 710 may generate motor set point signals. The motor set point signals may be provided to various motor controllers 708a-708e. The motor controllers 708a-708e may comprise one or more circuits configured to provide motor drive signals to the motors 704a-704e to drive the motors 704a-704e as described herein. In some examples, the motors 704a-704e may be brushed DC electric motors. For example, the velocity of the motors 704a-704e may be proportional to the respective motor drive signals. In some examples, the motors 704a-704e may be brushless DC electric motors, and the respective motor drive signals may comprise a PWM signal provided to one or more stator windings of the motors 704a-704e. Also, in some examples, the motor controllers 708a-708e may be omitted and the control circuit 710 may generate the motor drive signals directly.

In one aspect, the control circuit 710 may initially operate each of the motors 704a-704e in an open-loop configuration for a first open-loop portion of a stroke of the displacement member. Based on the response of the robotic surgical instrument 700 during the open-loop portion of the stroke, the control circuit 710 may select a firing control program in a closed-loop configuration. The response of the instrument may include a translation distance of the displacement member during the open-loop portion, a time elapsed during the open-loop portion, the energy provided to one of the motors 704a-704e during the open-loop portion, a sum of pulse widths of a motor drive signal, etc. After the open-loop portion, the control circuit 710 may implement the selected firing control program for a second portion of the displacement member stroke. For example, during a closed-loop portion of the stroke, the control circuit 710 may modulate one of the motors 704a-704e based on translation data describing a position of the displacement member in a closed-loop manner to translate the displacement member at a constant velocity.

In one aspect, the motors 704a-704e may receive power from an energy source 712. The energy source 712 may be a DC power supply driven by a main alternating current power source, a battery, a super capacitor, or any other suitable energy source. The motors 704a-704e may be mechanically coupled to individual movable mechanical elements such as the I-beam 714, anvil 716, shaft 740, articulation 742a, and articulation 742b via respective transmissions 706a-706e. The transmissions 706a-706e may include one or more gears or other linkage components to couple the motors 704a-704e to movable mechanical elements. A position sensor 734 may sense a position of the I-beam 714. The position sensor 734 may be or include any type of sensor that is capable of generating position data that indicate a position of the I-beam 714. In some examples, the position sensor 734 may include an encoder configured to provide a series of pulses to the control circuit 710 as the I-beam 714 translates distally and proximally. The control circuit 710 may track the pulses to determine the position of the I-beam 714. Other suitable position sensors may be used, including, for example, a proximity sensor. Other types of position sensors may provide other signals indicating motion of the I-beam 714. Also, in some examples, the position sensor 734 may be omitted. Where any of the motors 704a-704e is a stepper motor, the control circuit 710 may track the position of the I-beam 714 by aggregating the number and direction of steps that the motor 704 has been instructed to execute. The position sensor 734 may be located in the end effector 702 or at any other portion of the instrument. The outputs of each of the motors 704a-704e include a torque sensor 744a-744e to sense force and have an encoder to sense rotation of the drive shaft.

In one aspect, the control circuit 710 is configured to drive a firing member such as the I-beam 714 portion of the end effector 702. The control circuit 710 provides a motor set point to a motor control 708a, which provides a drive signal to the motor 704a. The output shaft of the motor 704a is coupled to a torque sensor 744a. The torque sensor 744a is coupled to a transmission 706a which is coupled to the I-beam 714. The transmission 706a comprises movable mechanical elements such as rotating elements and a firing member to control the movement of the I-beam 714 distally and proximally along a longitudinal axis of the end effector 702. In one aspect, the motor 704a may be coupled to the knife gear assembly, which includes a knife gear reduction set that includes a first knife drive gear and a second knife drive gear. A torque sensor 744a provides a firing force feedback signal to the control circuit 710. The firing force signal represents the force required to fire or displace the I-beam 714. A position sensor 734 may be configured to provide the position of the I-beam 714 along the firing stroke or the position of the firing member as a feedback signal to the control circuit 710. The end effector 702 may include additional sensors 738 configured to provide feedback signals to the control circuit 710. When ready to use, the control circuit 710 may provide a firing signal to the motor control 708a. In response to the firing signal, the motor 704a may drive the firing member distally along the longitudinal axis of the end effector 702 from a proximal stroke start position to a stroke end position distal to the stroke start position. As the firing member translates distally, an I-beam 714, with a cutting element positioned at a distal end, advances distally to cut tissue located between the staple cartridge 718 and the anvil 716.

In one aspect, the control circuit 710 is configured to drive a closure member such as the anvil 716 portion of the end effector 702. The control circuit 710 provides a motor set point to a motor control 708b, which provides a drive signal to the motor 704b. The output shaft of the motor 704b is coupled to a torque sensor 744b. The torque sensor 744b is coupled to a transmission 706b which is coupled to the anvil 716. The transmission 706b comprises movable mechanical elements such as rotating elements and a closure member to control the movement of the anvil 716 from the open and closed positions. In one aspect, the motor 704b is coupled to a closure gear assembly, which includes a closure reduction gear set that is supported in meshing engagement with the closure spur gear. The torque sensor 744b provides a closure force feedback signal to the control circuit 710. The closure force feedback signal represents the closure force applied to the anvil 716. The position sensor 734 may be configured to provide the position of the closure member as a feedback signal to the control circuit 710. Additional sensors 738 in the end effector 702 may provide the closure force feedback signal to the control circuit 710. The pivotable anvil 716 is positioned opposite the staple cartridge 718. When ready to use, the control circuit 710 may provide a closure signal to the motor control 708b. In response to the closure signal, the motor 704b advances a closure member to grasp tissue between the anvil 716 and the staple cartridge 718.

In one aspect, the control circuit 710 is configured to rotate a shaft member such as the shaft 740 to rotate the end effector 702. The control circuit 710 provides a motor set point to a motor control 708c, which provides a drive signal to the motor 704c. The output shaft of the motor 704c is coupled to a torque sensor 744c. The torque sensor 744c is coupled to a transmission 706c which is coupled to the shaft 740. The transmission 706c comprises movable mechanical elements such as rotating elements to control the rotation of the shaft 740 clockwise or counterclockwise up to and over 360°. In one aspect, the motor 704c is coupled to the rotational transmission assembly, which includes a tube gear segment that is formed on (or attached to) the proximal end of the proximal closure tube for operable engagement by a rotational gear assembly that is operably supported on the tool mounting plate. The torque sensor 744c provides a rotation force feedback signal to the control circuit 710. The rotation force feedback signal represents the rotation force applied to the shaft 740. The position sensor 734 may be configured to provide the position of the closure member as a feedback signal to the control circuit 710. Additional sensors 738 such as a shaft encoder may provide the rotational position of the shaft 740 to the control circuit 710.

In one aspect, the control circuit 710 is configured to articulate the end effector 702. The control circuit 710 provides a motor set point to a motor control 708d, which provides a drive signal to the motor 704d. The output shaft of the motor 704d is coupled to a torque sensor 744d. The torque sensor 744d is coupled to a transmission 706d which is coupled to an articulation member 742a. The transmission 706d comprises movable mechanical elements such as articulation elements to control the articulation of the end effector 702 ±65°. In one aspect, the motor 704d is coupled to an articulation nut, which is rotatably journaled on the proximal end portion of the distal spine portion and is rotatably driven thereon by an articulation gear assembly. The torque sensor 744d provides an articulation force feedback signal to the control circuit 710. The articulation force feedback signal represents the articulation force applied to the end effector 702. Sensors 738, such as an articulation encoder, may provide the articulation position of the end effector 702 to the control circuit 710.

In another aspect, the articulation function of the robotic surgical system 700 may comprise two articulation members, or links, 742a, 742b. These articulation members 742a, 742b are driven by separate disks on the robot interface (the rack) which are driven by the two motors 708d, 708e. When the separate firing motor 704a is provided, each of articulation links 742a, 742b can be antagonistically driven with respect to the other link in order to provide a resistive holding motion and a load to the head when it is not moving and to provide an articulation motion as the head is articulated. The articulation members 742a, 742b attach to the head at a fixed radius as the head is rotated. Accordingly, the mechanical advantage of the push-and-pull link changes as the head is rotated. This change in the mechanical advantage may be more pronounced with other articulation link drive systems.

In one aspect, the one or more motors 704a-704e may comprise a brushed DC motor with a gearbox and mechanical links to a firing member, closure member, or articulation member. Another example includes electric motors 704a-704e that operate the movable mechanical elements such as the displacement member, articulation links, closure tube, and shaft. An outside influence is an unmeasured, unpredictable influence of things like tissue, surrounding bodies, and friction on the physical system. Such outside influence can be referred to as drag, which acts in opposition to one of electric motors 704a-704e. The outside influence, such as drag, may cause the operation of the physical system to deviate from a desired operation of the physical system.

In one aspect, the position sensor 734 may be implemented as an absolute positioning system. In one aspect, the position sensor 734 may comprise a magnetic rotary absolute positioning system implemented as an AS5055EQFT single-chip magnetic rotary position sensor available from Austria Microsystems, AG. The position sensor 734 may interface with the control circuit 710 to provide an absolute positioning system. The position may include multiple Hall-effect elements located above a magnet and coupled to a CORDIC processor, also known as the digit-by-digit method and Volder's algorithm, that is provided to implement a simple and efficient algorithm to calculate hyperbolic and trigonometric functions that require only addition, subtraction, bitshift, and table lookup operations.

In one aspect, the control circuit 710 may be in communication with one or more sensors 738. The sensors 738 may be positioned on the end effector 702 and adapted to operate with the robotic surgical instrument 700 to measure the various derived parameters such as the gap distance versus time, tissue compression versus time, and anvil strain versus time. The sensors 738 may comprise a magnetic sensor, a magnetic field sensor, a strain gauge, a load cell, a pressure sensor, a force sensor, a torque sensor, an inductive sensor such as an eddy current sensor, a resistive sensor, a capacitive sensor, an optical sensor, and/or any other suitable sensor for measuring one or more parameters of the end effector 702. The sensors 738 may include one or more sensors. The sensors 738 may be located on the staple cartridge 718 deck to determine tissue location using segmented electrodes. The torque sensors 744a-744e may be configured to sense force such as firing force, closure force, and/or articulation force, among others. Accordingly, the control circuit 710 can sense (1) the closure load experienced by the distal closure tube and its position, (2) the firing member at the rack and its position, (3) what portion of the staple cartridge 718 has tissue on it, and (4) the load and position on both articulation rods.

In one aspect, the one or more sensors 738 may comprise a strain gauge, such as a micro-strain gauge, configured to measure the magnitude of the strain in the anvil 716 during a clamped condition. The strain gauge provides an electrical signal whose amplitude varies with the magnitude of the strain. The sensors 738 may comprise a pressure sensor configured to detect a pressure generated by the presence of compressed tissue between the anvil 716 and the staple cartridge 718. The sensors 738 may be configured to detect impedance of a tissue section located between the anvil 716 and the staple cartridge 718 that is indicative of the thickness and/or fullness of tissue located therebetween.

In one aspect, the sensors 738 may be implemented as one or more limit switches, electromechanical devices, solid-state switches, Hall-effect devices, magneto-resistive (MR) devices, giant magneto-resistive (GMR) devices, magnetometers, among others. In other implementations, the sensors 738 may be implemented as solid-state switches that operate under the influence of light, such as optical sensors, IR sensors, ultraviolet sensors, among others. Still, the switches may be solid-state devices such as transistors (e.g., FET, junction FET, MOSFET, bipolar, and the like). In other implementations, the sensors 738 may include electrical conductorless switches, ultrasonic switches, accelerometers, and inertial sensors, among others.

In one aspect, the sensors 738 may be configured to measure forces exerted on the anvil 716 by the closure drive system. For example, one or more sensors 738 can be at an interaction point between the closure tube and the anvil 716 to detect the closure forces applied by the closure tube to the anvil 716. The forces exerted on the anvil 716 can be representative of the tissue compression experienced by the tissue section captured between the anvil 716 and the staple cartridge 718. The one or more sensors 738 can be positioned at various interaction points along the closure drive system to detect the closure forces applied to the anvil 716 by the closure drive system. The one or more sensors 738 may be sampled in real time during a clamping operation by the processor of the control circuit 710. The control circuit 710 receives real-time sample measurements to provide and analyze time-based information and assess, in real time, closure forces applied to the anvil 716.

In one aspect, a current sensor 736 can be employed to measure the current drawn by each of the motors 704a-704e. The force required to advance any of the movable mechanical elements such as the I-beam 714 corresponds to the current drawn by one of the motors 704a-704e. The force is converted to a digital signal and provided to the control circuit 710. The control circuit 710 can be configured to simulate the response of the actual system of the instrument in the software of the controller. A displacement member can be actuated to move an I-beam 714 in the end effector 702 at or near a target velocity. The robotic surgical instrument 700 can include a feedback controller, which can be one of any feedback controllers, including, but not limited to a PID, a state feedback, a linear-quadratic (LQR), and/or an adaptive controller, for example. The robotic surgical instrument 700 can include a power source to convert the signal from the feedback controller into a physical input such as case voltage, PWM voltage, frequency modulated voltage, current, torque, and/or force, for example. Additional details are disclosed in U.S. patent application Ser. No. 15/636,829, titled CLOSED LOOP VELOCITY CONTROL TECHNIQUES FOR ROBOTIC SURGICAL INSTRUMENT, filed Jun. 29, 2017, which is herein incorporated by reference in its entirety.

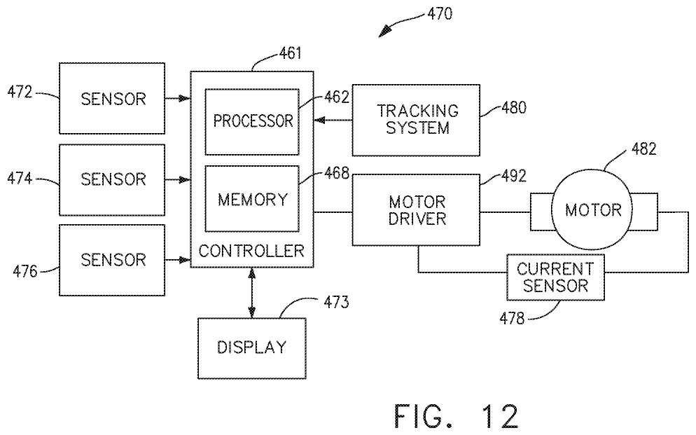

FIG. 18 illustrates a block diagram of a surgical instrument 750 programmed to control the distal translation of a displacement member according to one aspect of this disclosure. In one aspect, the surgical instrument 750 is programmed to control the distal translation of a displacement member such as the I-beam 764. The surgical instrument 750 comprises an end effector 752 that may comprise an anvil 766, an I-beam 764 (including a sharp cutting edge), and a removable staple cartridge 768.

The position, movement, displacement, and/or translation of a linear displacement member, such as the I-beam 764, can be measured by an absolute positioning system, sensor arrangement, and position sensor 784. Because the I-beam 764 is coupled to a longitudinally movable drive member, the position of the I-beam 764 can be determined by measuring the position of the longitudinally movable drive member employing the position sensor 784. Accordingly, in the following description, the position, displacement, and/or translation of the I-beam 764 can be achieved by the position sensor 784 as described herein. A control circuit 760 may be programmed to control the translation of the displacement member, such as the I-beam 764. The control circuit 760, in some examples, may comprise one or more microcontrollers, microprocessors, or other suitable processors for executing instructions that cause the processor or processors to control the displacement member, e.g., the I-beam 764, in the manner described. In one aspect, a timer/counter 781 provides an output signal, such as the elapsed time or a digital count, to the control circuit 760 to correlate the position of the I-beam 764 as determined by the position sensor 784 with the output of the timer/counter 781 such that the control circuit 760 can determine the position of the I-beam 764 at a specific time (t) relative to a starting position. The timer/counter 781 may be configured to measure elapsed time, count external events, or time external events.

The control circuit 760 may generate a motor set point signal 772. The motor set point signal 772 may be provided to a motor controller 758. The motor controller 758 may comprise one or more circuits configured to provide a motor drive signal 774 to the motor 754 to drive the motor 754 as described herein. In some examples, the motor 754 may be a brushed DC electric motor. For example, the velocity of the motor 754 may be proportional to the motor drive signal 774. In some examples, the motor 754 may be a brushless DC electric motor and the motor drive signal 774 may comprise a PWM signal provided to one or more stator windings of the motor 754. Also, in some examples, the motor controller 758 may be omitted, and the control circuit 760 may generate the motor drive signal 774 directly.

The motor 754 may receive power from an energy source 762. The energy source 762 may be or include a battery, a super capacitor, or any other suitable energy source. The motor 754 may be mechanically coupled to the I-beam 764 via a transmission 756. The transmission 756 may include one or more gears or other linkage components to couple the motor 754 to the I-beam 764. A position sensor 784 may sense a position of the I-beam 764. The position sensor 784 may be or include any type of sensor that is capable of generating position data that indicate a position of the I-beam 764. In some examples, the position sensor 784 may include an encoder configured to provide a series of pulses to the control circuit 760 as the I-beam 764 translates distally and proximally. The control circuit 760 may track the pulses to determine the position of the I-beam 764. Other suitable position sensors may be used, including, for example, a proximity sensor. Other types of position sensors may provide other signals indicating motion of the I-beam 764. Also, in some examples, the position sensor 784 may be omitted. Where the motor 754 is a stepper motor, the control circuit 760 may track the position of the I-beam 764 by aggregating the number and direction of steps that the motor 754 has been instructed to execute. The position sensor 784 may be located in the end effector 752 or at any other portion of the instrument.

The control circuit 760 may be in communication with one or more sensors 788. The sensors 788 may be positioned on the end effector 752 and adapted to operate with the surgical instrument 750 to measure the various derived parameters such as gap distance versus time, tissue compression versus time, and anvil strain versus time. The sensors 788 may comprise a magnetic sensor, a magnetic field sensor, a strain gauge, a pressure sensor, a force sensor, an inductive sensor such as an eddy current sensor, a resistive sensor, a capacitive sensor, an optical sensor, and/or any other suitable sensor for measuring one or more parameters of the end effector 752. The sensors 788 may include one or more sensors.

The one or more sensors 788 may comprise a strain gauge, such as a micro-strain gauge, configured to measure the magnitude of the strain in the anvil 766 during a clamped condition. The strain gauge provides an electrical signal whose amplitude varies with the magnitude of the strain. The sensors 788 may comprise a pressure sensor configured to detect a pressure generated by the presence of compressed tissue between the anvil 766 and the staple cartridge 768. The sensors 788 may be configured to detect impedance of a tissue section located between the anvil 766 and the staple cartridge 768 that is indicative of the thickness and/or fullness of tissue located therebetween.

The sensors 788 may be is configured to measure forces exerted on the anvil 766 by a closure drive system. For example, one or more sensors 788 can be at an interaction point between a closure tube and the anvil 766 to detect the closure forces applied by a closure tube to the anvil 766. The forces exerted on the anvil 766 can be representative of the tissue compression experienced by the tissue section captured between the anvil 766 and the staple cartridge 768. The one or more sensors 788 can be positioned at various interaction points along the closure drive system to detect the closure forces applied to the anvil 766 by the closure drive system. The one or more sensors 788 may be sampled in real time during a clamping operation by a processor of the control circuit 760. The control circuit 760 receives real-time sample measurements to provide and analyze time-based information and assess, in real time, closure forces applied to the anvil 766.

A current sensor 786 can be employed to measure the current drawn by the motor 754. The force required to advance the I-beam 764 corresponds to the current drawn by the motor 754. The force is converted to a digital signal and provided to the control circuit 760.

The control circuit 760 can be configured to simulate the response of the actual system of the instrument in the software of the controller. A displacement member can be actuated to move an I-beam 764 in the end effector 752 at or near a target velocity. The surgical instrument 750 can include a feedback controller, which can be one of any feedback controllers, including, but not limited to a PID, a state feedback, LQR, and/or an adaptive controller, for example. The surgical instrument 750 can include a power source to convert the signal from the feedback controller into a physical input such as case voltage, PWM voltage, frequency modulated voltage, current, torque, and/or force, for example.

The actual drive system of the surgical instrument 750 is configured to drive the displacement member, cutting member, or I-beam 764, by a brushed DC motor with gearbox and mechanical links to an articulation and/or knife system. Another example is the electric motor 754 that operates the displacement member and the articulation driver, for example, of an interchangeable shaft assembly. An outside influence is an unmeasured, unpredictable influence of things like tissue, surrounding bodies and friction on the physical system. Such outside influence can be referred to as drag which acts in opposition to the electric motor 754. The outside influence, such as drag, may cause the operation of the physical system to deviate from a desired operation of the physical system.

Various example aspects are directed to a surgical instrument 750 comprising an end effector 752 with motor-driven surgical stapling and cutting implements. For example, a motor 754 may drive a displacement member distally and proximally along a longitudinal axis of the end effector 752. The end effector 752 may comprise a pivotable anvil 766 and, when configured for use, a staple cartridge 768 positioned opposite the anvil 766. A clinician may grasp tissue between the anvil 766 and the staple cartridge 768, as described herein. When ready to use the instrument 750, the clinician may provide a firing signal, for example by depressing a trigger of the instrument 750. In response to the firing signal, the motor 754 may drive the displacement member distally along the longitudinal axis of the end effector 752 from a proximal stroke begin position to a stroke end position distal of the stroke begin position. As the displacement member translates distally, an I-beam 764 with a cutting element positioned at a distal end, may cut the tissue between the staple cartridge 768 and the anvil 766.

In various examples, the surgical instrument 750 may comprise a control circuit 760 programmed to control the distal translation of the displacement member, such as the I-beam 764, for example, based on one or more tissue conditions. The control circuit 760 may be programmed to sense tissue conditions, such as thickness, either directly or indirectly, as described herein. The control circuit 760 may be programmed to select a firing control program based on tissue conditions. A firing control program may describe the distal motion of the displacement member. Different firing control programs may be selected to better treat different tissue conditions. For example, when thicker tissue is present, the control circuit 760 may be programmed to translate the displacement member at a lower velocity and/or with lower power. When thinner tissue is present, the control circuit 760 may be programmed to translate the displacement member at a higher velocity and/or with higher power.

In some examples, the control circuit 760 may initially operate the motor 754 in an open loop configuration for a first open loop portion of a stroke of the displacement member. Based on a response of the instrument 750 during the open loop portion of the stroke, the control circuit 760 may select a firing control program. The response of the instrument may include, a translation distance of the displacement member during the open loop portion, a time elapsed during the open loop portion, energy provided to the motor 754 during the open loop portion, a sum of pulse widths of a motor drive signal, etc. After the open loop portion, the control circuit 760 may implement the selected firing control program for a second portion of the displacement member stroke. For example, during the closed loop portion of the stroke, the control circuit 760 may modulate the motor 754 based on translation data describing a position of the displacement member in a closed loop manner to translate the displacement member at a constant velocity. Additional details are disclosed in U.S. patent application Ser. No. 15/720,852, titled SYSTEM AND METHODS FOR CONTROLLING A DISPLAY OF A SURGICAL INSTRUMENT, filed Sep. 29, 2017, which is herein incorporated by reference in its entirety.

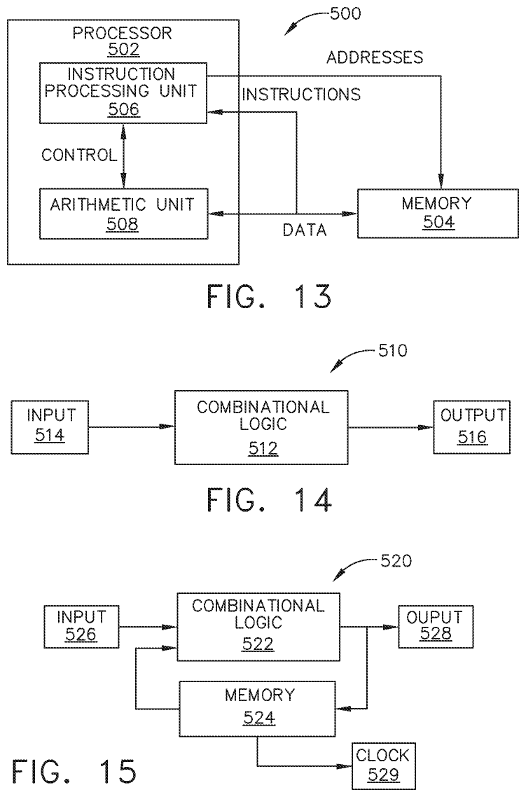

FIG. 19 is a schematic diagram of a surgical instrument 790 configured to control various functions according to one aspect of this disclosure. In one aspect, the surgical instrument 790 is programmed to control distal translation of a displacement member such as the I-beam 764. The surgical instrument 790 comprises an end effector 792 that may comprise an anvil 766, an I-beam 764, and a removable staple cartridge 768 which may be interchanged with an RF cartridge 796 (shown in dashed line).

In one aspect, sensors 788 may be implemented as a limit switch, electromechanical device, solid-state switches, Hall-effect devices, MR devices, GMR devices, magnetometers, among others. In other implementations, the sensors 638 may be solid-state switches that operate under the influence of light, such as optical sensors, IR sensors, ultraviolet sensors, among others. Still, the switches may be solid-state devices such as transistors (e.g., FET, junction FET, MOSFET, bipolar, and the like). In other implementations, the sensors 788 may include electrical conductorless switches, ultrasonic switches, accelerometers, and inertial sensors, among others.

In one aspect, the position sensor 784 may be implemented as an absolute positioning system comprising a magnetic rotary absolute positioning system implemented as an AS5055EQFT single-chip magnetic rotary position sensor available from Austria Microsystems, AG. The position sensor 784 may interface with the control circuit 760 to provide an absolute positioning system. The position may include multiple Hall-effect elements located above a magnet and coupled to a CORDIC processor, also known as the digit-by-digit method and Volder's algorithm, that is provided to implement a simple and efficient algorithm to calculate hyperbolic and trigonometric functions that require only addition, subtraction, bitshift, and table lookup operations.

In one aspect, the I-beam 764 may be implemented as a knife member comprising a knife body that operably supports a tissue cutting blade thereon and may further include anvil engagement tabs or features and channel engagement features or a foot. In one aspect, the staple cartridge 768 may be implemented as a standard (mechanical) surgical fastener cartridge. In one aspect, the RF cartridge 796 may be implemented as an RF cartridge. These and other sensors arrangements are described in commonly owned U.S. patent application Ser. No. 15/628,175, titled TECHNIQUES FOR ADAPTIVE CONTROL OF MOTOR VELOCITY OF A SURGICAL STAPLING AND CUTTING INSTRUMENT, filed Jun. 20, 2017, which is herein incorporated by reference in its entirety.

The position, movement, displacement, and/or translation of a linear displacement member, such as the I-beam 764, can be measured by an absolute positioning system, sensor arrangement, and position sensor represented as position sensor 784. Because the I-beam 764 is coupled to the longitudinally movable drive member, the position of the !-beam 764 can be determined by measuring the position of the longitudinally movable drive member employing the position sensor 784. Accordingly, in the following description, the position, displacement, and/or translation of the I-beam 764 can be achieved by the position sensor 784 as described herein. A control circuit 760 may be programmed to control the translation of the displacement member, such as the I-beam 764, as described herein. The control circuit 760, in some examples, may comprise one or more microcontrollers, microprocessors, or other suitable processors for executing instructions that cause the processor or processors to control the displacement member, e.g., the I-beam 764, in the manner described. In one aspect, a timer/counter 781 provides an output signal, such as the elapsed time or a digital count, to the control circuit 760 to correlate the position of the I-beam 764 as determined by the position sensor 784 with the output of the timer/counter 781 such that the control circuit 760 can determine the position of the I-beam 764 at a specific time (t) relative to a starting position. The timer/counter 781 may be configured to measure elapsed time, count external events, or time external events.

The control circuit 760 may generate a motor set point signal 772. The motor set point signal 772 may be provided to a motor controller 758. The motor controller 758 may comprise one or more circuits configured to provide a motor drive signal 774 to the motor 754 to drive the motor 754 as described herein. In some examples, the motor 754 may be a brushed DC electric motor. For example, the velocity of the motor 754 may be proportional to the motor drive signal 774. In some examples, the motor 754 may be a brushless DC electric motor and the motor drive signal 774 may comprise a PWM signal provided to one or more stator windings of the motor 754. Also, in some examples, the motor controller 758 may be omitted, and the control circuit 760 may generate the motor drive signal 774 directly.

The motor 754 may receive power from an energy source 762. The energy source 762 may be or include a battery, a super capacitor, or any other suitable energy source. The motor 754 may be mechanically coupled to the I-beam 764 via a transmission 756. The transmission 756 may include one or more gears or other linkage components to couple the motor 754 to the I-beam 764. A position sensor 784 may sense a position of the I-beam 764. The position sensor 784 may be or include any type of sensor that is capable of generating position data that indicate a position of the I-beam 764. In some examples, the position sensor 784 may include an encoder configured to provide a series of pulses to the control circuit 760 as the I-beam 764 translates distally and proximally. The control circuit 760 may track the pulses to determine the position of the I-beam 764. Other suitable position sensors may be used, including, for example, a proximity sensor. Other types of position sensors may provide other signals indicating motion of the I-beam 764. Also, in some examples, the position sensor 784 may be omitted. Where the motor 754 is a stepper motor, the control circuit 760 may track the position of the I-beam 764 by aggregating the number and direction of steps that the motor has been instructed to execute. The position sensor 784 may be located in the end effector 792 or at any other portion of the instrument.

The control circuit 760 may be in communication with one or more sensors 788. The sensors 788 may be positioned on the end effector 792 and adapted to operate with the surgical instrument 790 to measure the various derived parameters such as gap distance versus time, tissue compression versus time, and anvil strain versus time. The sensors 788 may comprise a magnetic sensor, a magnetic field sensor, a strain gauge, a pressure sensor, a force sensor, an inductive sensor such as an eddy current sensor, a resistive sensor, a capacitive sensor, an optical sensor, and/or any other suitable sensor for measuring one or more parameters of the end effector 792. The sensors 788 may include one or more sensors.

The one or more sensors 788 may comprise a strain gauge, such as a micro-strain gauge, configured to measure the magnitude of the strain in the anvil 766 during a clamped condition. The strain gauge provides an electrical signal whose amplitude varies with the magnitude of the strain. The sensors 788 may comprise a pressure sensor configured to detect a pressure generated by the presence of compressed tissue between the anvil 766 and the staple cartridge 768. The sensors 788 may be configured to detect impedance of a tissue section located between the anvil 766 and the staple cartridge 768 that is indicative of the thickness and/or fullness of tissue located therebetween.

The sensors 788 may be is configured to measure forces exerted on the anvil 766 by the closure drive system. For example, one or more sensors 788 can be at an interaction point between a closure tube and the anvil 766 to detect the closure forces applied by a closure tube to the anvil 766. The forces exerted on the anvil 766 can be representative of the tissue compression experienced by the tissue section captured between the anvil 766 and the staple cartridge 768. The one or more sensors 788 can be positioned at various interaction points along the closure drive system to detect the closure forces applied to the anvil 766 by the closure drive system. The one or more sensors 788 may be sampled in real time during a clamping operation by a processor portion of the control circuit 760. The control circuit 760 receives real-time sample measurements to provide and analyze time-based information and assess, in real time, closure forces applied to the anvil 766.

A current sensor 786 can be employed to measure the current drawn by the motor 754. The force required to advance the I-beam 764 corresponds to the current drawn by the motor 754. The force is converted to a digital signal and provided to the control circuit 760.

An RF energy source 794 is coupled to the end effector 792 and is applied to the RF cartridge 796 when the RF cartridge 796 is loaded in the end effector 792 in place of the staple cartridge 768. The control circuit 760 controls the delivery of the RF energy to the RF cartridge 796.

Additional details are disclosed in U.S. patent application Ser. No. 15/636,096, titled SURGICAL SYSTEM COUPLABLE WITH STAPLE CARTRIDGE AND RADIO FREQUENCY CARTRIDGE, AND METHOD OF USING SAME, filed Jun. 28, 2017, which is herein incorporated by reference in its entirety.

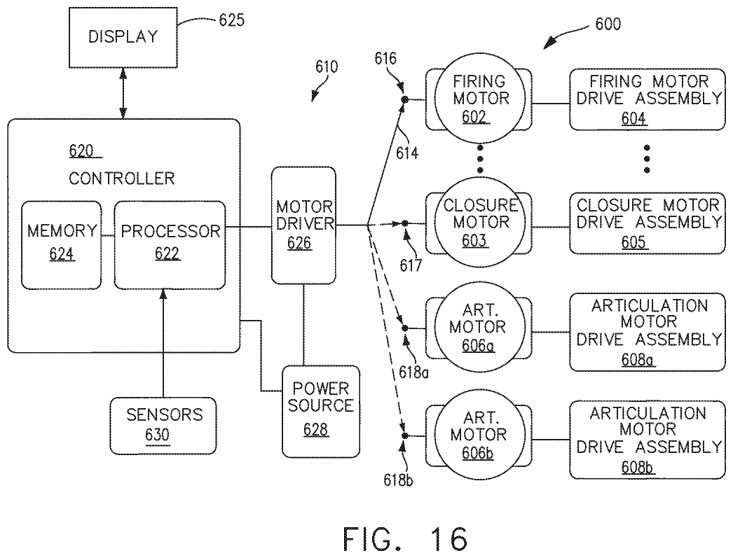

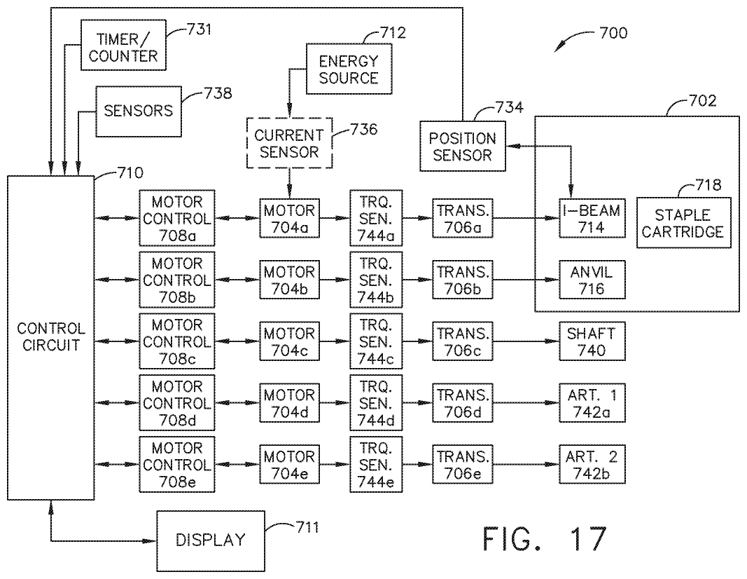

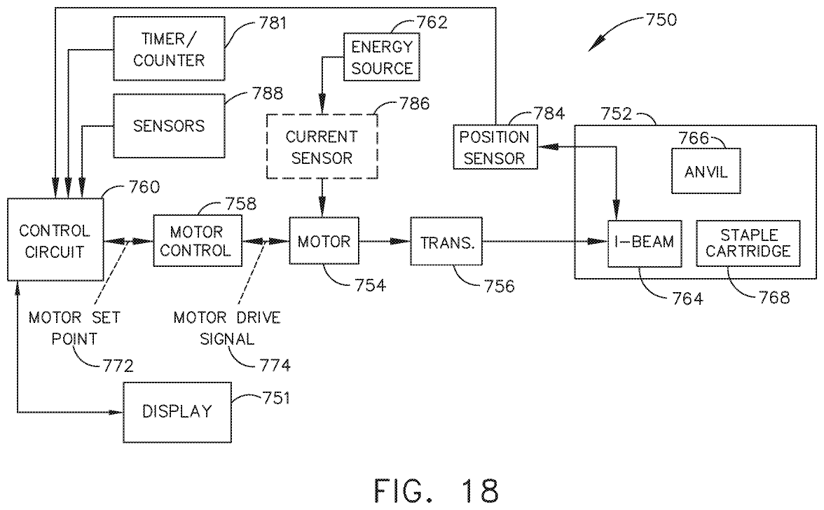

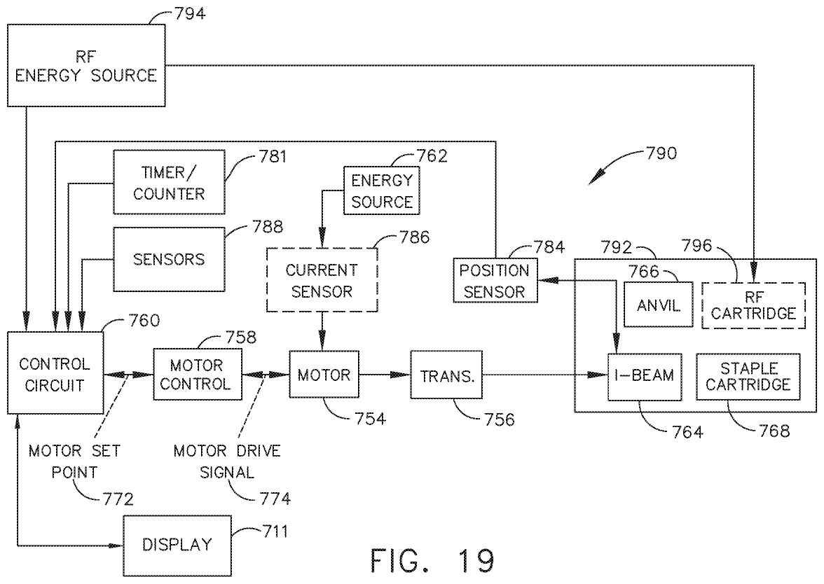

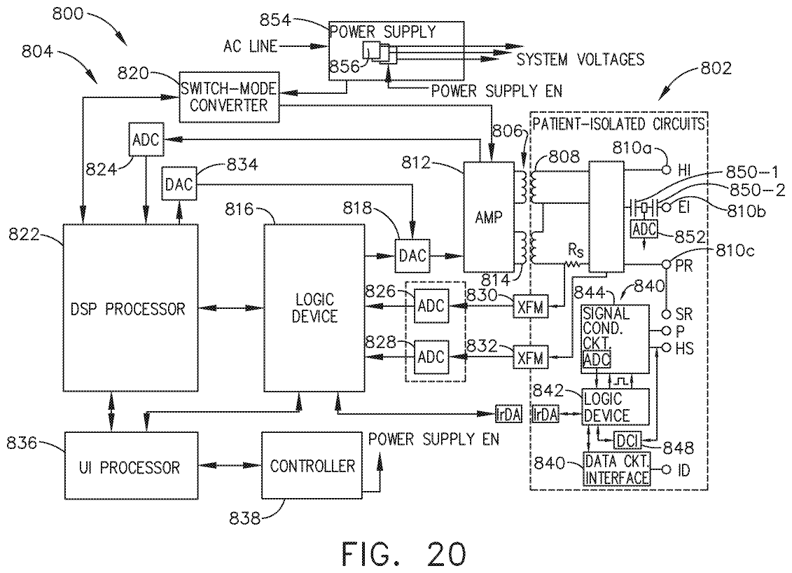

FIG. 20 is a simplified block diagram of a generator 800 configured to provide inductorless tuning, among other benefits. Additional details of the generator 800 are described in U.S. Pat. No. 9,060,775, titled SURGICAL GENERATOR FOR ULTRASONIC AND ELECTROSURGICAL DEVICES, which issued on Jun. 23, 2015, which is herein incorporated by reference in its entirety. The generator 800 may comprise a patient isolated stage 802 in communication with a non-isolated stage 804 via a power transformer 806. A secondary winding 808 of the power transformer 806 is contained in the isolated stage 802 and may comprise a tapped configuration (e.g., a center-tapped or a non-center-tapped configuration) to define drive signal outputs 810a, 810b, 810c for delivering drive signals to different surgical instruments, such as, for example, an ultrasonic surgical instrument, an RF electrosurgical instrument, and a multifunction surgical instrument which includes ultrasonic and RF energy modes that can be delivered alone or simultaneously. In particular, drive signal outputs 810a, 810c may output an ultrasonic drive signal (e.g., a 420V root-mean-square (RMS) drive signal) to an ultrasonic surgical instrument, and drive signal outputs 810b, 810c may output an RF electrosurgical drive signal (e.g., a 100V RMS drive signal) to an RF electrosurgical instrument, with the drive signal output 810b corresponding to the center tap of the power transformer 806.

In certain forms, the ultrasonic and electrosurgical drive signals may be provided simultaneously to distinct surgical instruments and/or to a single surgical instrument, such as the multifunction surgical instrument, having the capability to deliver both ultrasonic and electrosurgical energy to tissue. It will be appreciated that the electrosurgical signal, provided either to a dedicated electrosurgical instrument and/or to a combined multifunction ultrasonic/electrosurgical instrument may be either a therapeutic or sub-therapeutic level signal where the sub-therapeutic signal can be used, for example, to monitor tissue or instrument conditions and provide feedback to the generator. For example, the ultrasonic and RF signals can be delivered separately or simultaneously from a generator with a single output port in order to provide the desired output signal to the surgical instrument, as will be discussed in more detail below. Accordingly, the generator can combine the ultrasonic and electrosurgical RF energies and deliver the combined energies to the multifunction ultrasonic/electrosurgical instrument. Bipolar electrodes can be placed on one or both jaws of the end effector. One jaw may be driven by ultrasonic energy in addition to electrosurgical RF energy, working simultaneously. The ultrasonic energy may be employed to dissect tissue, while the electrosurgical RF energy may be employed for vessel sealing.

The non-isolated stage 804 may comprise a power amplifier 812 having an output connected to a primary winding 814 of the power transformer 806. In certain forms, the power amplifier 812 may comprise a push-pull amplifier. For example, the non-isolated stage 804 may further comprise a logic device 816 for supplying a digital output to a digital-to-analog converter (DAC) circuit 818, which in turn supplies a corresponding analog signal to an input of the power amplifier 812. In certain forms, the logic device 816 may comprise a programmable gate array (PGA), a FPGA, programmable logic device (PLD), among other logic circuits, for example. The logic device 816, by virtue of controlling the input of the power amplifier 812 via the DAC circuit 818, may therefore control any of a number of parameters (e.g., frequency, waveform shape, waveform amplitude) of drive signals appearing at the drive signal outputs 810a, 810b, 810c. In certain forms and as discussed below, the logic device 816, in conjunction with a processor (e.g., a DSP discussed below), may implement a number of DSP-based and/or other control algorithms to control parameters of the drive signals output by the generator 800.

Power may be supplied to a power rail of the power amplifier 812 by a switch-mode regulator 820, e.g., a power converter. In certain forms, the switch-mode regulator 820 may comprise an adjustable buck regulator, for example. The non-isolated stage 804 may further comprise a first processor 822, which in one form may comprise a DSP processor such as an Analog Devices ADSP-21469 SHARC DSP, available from Analog Devices, Norwood, Mass., for example, although in various forms any suitable processor may be employed. In certain forms the DSP processor 822 may control the operation of the switch-mode regulator 820 responsive to voltage feedback data received from the power amplifier 812 by the DSP processor 822 via an ADC circuit 824. In one form, for example, the DSP processor 822 may receive as input, via the ADC circuit 824, the waveform envelope of a signal (e.g., an RF signal) being amplified by the power amplifier 812. The DSP processor 822 may then control the switch-mode regulator 820 (e.g., via a PWM output) such that the rail voltage supplied to the power amplifier 812 tracks the waveform envelope of the amplified signal. By dynamically modulating the rail voltage of the power amplifier 812 based on the waveform envelope, the efficiency of the power amplifier 812 may be significantly improved relative to a fixed rail voltage amplifier schemes.

In certain forms, the logic device 816, in conjunction with the DSP processor 822, may implement a digital synthesis circuit such as a direct digital synthesizer control scheme to control the waveform shape, frequency, and/or amplitude of drive signals output by the generator 800. In one form, for example, the logic device 816 may implement a DDS control algorithm by recalling waveform samples stored in a dynamically updated lookup table (LUT), such as a RAM LUT, which may be embedded in an FPGA. This control algorithm is particularly useful for ultrasonic applications in which an ultrasonic transducer, such as an ultrasonic transducer, may be driven by a clean sinusoidal current at its resonant frequency. Because other frequencies may excite parasitic resonances, minimizing or reducing the total distortion of the motional branch current may correspondingly minimize or reduce undesirable resonance effects. Because the waveform shape of a drive signal output by the generator 800 is impacted by various sources of distortion present in the output drive circuit (e.g., the power transformer 806, the power amplifier 812), voltage and current feedback data based on the drive signal may be input into an algorithm, such as an error control algorithm implemented by the DSP processor 822, which compensates for distortion by suitably pre-distorting or modifying the waveform samples stored in the LUT on a dynamic, ongoing basis (e.g., in real time). In one form, the amount or degree of pre-distortion applied to the LUT samples may be based on the error between a computed motional branch current and a desired current waveform shape, with the error being determined on a sample-by-sample basis. In this way, the pre-distorted LUT samples, when processed through the drive circuit, may result in a motional branch drive signal having the desired waveform shape (e.g., sinusoidal) for optimally driving the ultrasonic transducer. In such forms, the LUT waveform samples will therefore not represent the desired waveform shape of the drive signal, but rather the waveform shape that is required to ultimately produce the desired waveform shape of the motional branch drive signal when distortion effects are taken into account.

The non-isolated stage 804 may further comprise a first ADC circuit 826 and a second ADC circuit 828 coupled to the output of the power transformer 806 via respective isolation transformers 830, 832 for respectively sampling the voltage and current of drive signals output by the generator 800. In certain forms, the ADC circuits 826, 828 may be configured to sample at high speeds (e.g., 80 mega samples per second (MSPS)) to enable oversampling of the drive signals. In one form, for example, the sampling speed of the ADC circuits 826, 828 may enable approximately 200× (depending on frequency) oversampling of the drive signals. In certain forms, the sampling operations of the ADC circuit 826, 828 may be performed by a single ADC circuit receiving input voltage and current signals via a two-way multiplexer. The use of high-speed sampling in forms of the generator 800 may enable, among other things, calculation of the complex current flowing through the motional branch (which may be used in certain forms to implement DDS-based waveform shape control described above), accurate digital filtering of the sampled signals, and calculation of real power consumption with a high degree of precision. Voltage and current feedback data output by the ADC circuits 826, 828 may be received and processed (e.g., first-in-first-out (FIFO) buffer, multiplexer) by the logic device 816 and stored in data memory for subsequent retrieval by, for example, the DSP processor 822. As noted above, voltage and current feedback data may be used as input to an algorithm for pre-distorting or modifying LUT waveform samples on a dynamic and ongoing basis. In certain forms, this may require each stored voltage and current feedback data pair to be indexed based on, or otherwise associated with, a corresponding LUT sample that was output by the logic device 816 when the voltage and current feedback data pair was acquired. Synchronization of the LUT samples and the voltage and current feedback data in this manner contributes to the correct timing and stability of the pre-distortion algorithm.

In certain forms, the voltage and current feedback data may be used to control the frequency and/or amplitude (e.g., current amplitude) of the drive signals. In one form, for example, voltage and current feedback data may be used to determine impedance phase. The frequency of the drive signal may then be controlled to minimize or reduce the difference between the determined impedance phase and an impedance phase setpoint (e.g., 0°), thereby minimizing or reducing the effects of harmonic distortion and correspondingly enhancing impedance phase measurement accuracy. The determination of phase impedance and a frequency control signal may be implemented in the DSP processor 822, for example, with the frequency control signal being supplied as input to a DDS control algorithm implemented by the logic device 816.

In another form, for example, the current feedback data may be monitored in order to maintain the current amplitude of the drive signal at a current amplitude setpoint. The current amplitude setpoint may be specified directly or determined indirectly based on specified voltage amplitude and power setpoints. In certain forms, control of the current amplitude may be implemented by control algorithm, such as, for example, a proportional—integral—derivative (PID) control algorithm, in the DSP processor 822. Variables controlled by the control algorithm to suitably control the current amplitude of the drive signal may include, for example, the scaling of the LUT waveform samples stored in the logic device 816 and/or the full-scale output voltage of the DAC circuit 818 (which supplies the input to the power amplifier 812) via a DAC circuit 834.

The non-isolated stage 804 may further comprise a second processor 836 for providing, among other things user interface (UI) functionality. In one form, the UI processor 836 may comprise an Atmel AT91SAM9263 processor having an ARM 926EJ-S core, available from Atmel Corporation, San Jose, Calif., for example. Examples of UI functionality supported by the UI processor 836 may include audible and visual user feedback, communication with peripheral devices (e.g., via a USB interface), communication with a foot switch, communication with an input device (e.g., a touch screen display) and communication with an output device (e.g., a speaker). The UI processor 836 may communicate with the DSP processor 822 and the logic device 816 (e.g., via SPI buses). Although the UI processor 836 may primarily support UI functionality, it may also coordinate with the DSP processor 822 to implement hazard mitigation in certain forms. For example, the UI processor 836 may be programmed to monitor various aspects of user input and/or other inputs (e.g., touch screen inputs, foot switch inputs, temperature sensor inputs) and may disable the drive output of the generator 800 when an erroneous condition is detected.

In certain forms, both the DSP processor 822 and the UI processor 836, for example, may determine and monitor the operating state of the generator 800. For the DSP processor 822, the operating state of the generator 800 may dictate, for example, which control and/or diagnostic processes are implemented by the DSP processor 822. For the UI processor 836, the operating state of the generator 800 may dictate, for example, which elements of a UI (e.g., display screens, sounds) are presented to a user. The respective DSP and UI processors 822, 836 may independently maintain the current operating state of the generator 800 and recognize and evaluate possible transitions out of the current operating state. The DSP processor 822 may function as the master in this relationship and determine when transitions between operating states are to occur. The UI processor 836 may be aware of valid transitions between operating states and may confirm if a particular transition is appropriate. For example, when the DSP processor 822 instructs the UI processor 836 to transition to a specific state, the UI processor 836 may verify that requested transition is valid. In the event that a requested transition between states is determined to be invalid by the UI processor 836, the UI processor 836 may cause the generator 800 to enter a failure mode.

The non-isolated stage 804 may further comprise a controller 838 for monitoring input devices (e.g., a capacitive touch sensor used for turning the generator 800 on and off, a capacitive touch screen). In certain forms, the controller 838 may comprise at least one processor and/or other controller device in communication with the UI processor 836. In one form, for example, the controller 838 may comprise a processor (e.g., a Meg168 8-bit controller available from Atmel) configured to monitor user input provided via one or more capacitive touch sensors. In one form, the controller 838 may comprise a touch screen controller (e.g., a QT5480 touch screen controller available from Atmel) to control and manage the acquisition of touch data from a capacitive touch screen.

In certain forms, when the generator 800 is in a “power off” state, the controller 838 may continue to receive operating power (e.g., via a line from a power supply of the generator 800, such as the power supply 854 discussed below). In this way, the controller 838 may continue to monitor an input device (e.g., a capacitive touch sensor located on a front panel of the generator 800) for turning the generator 800 on and off. When the generator 800 is in the power off state, the controller 838 may wake the power supply (e.g., enable operation of one or more DC/DC voltage converters 856 of the power supply 854) if activation of the “on/off” input device by a user is detected. The controller 838 may therefore initiate a sequence for transitioning the generator 800 to a “power on” state. Conversely, the controller 838 may initiate a sequence for transitioning the generator 800 to the power off state if activation of the “on/off” input device is detected when the generator 800 is in the power on state. In certain forms, for example, the controller 838 may report activation of the “on/off” input device to the UI processor 836, which in turn implements the necessary process sequence for transitioning the generator 800 to the power off state. In such forms, the controller 838 may have no independent ability for causing the removal of power from the generator 800 after its power on state has been established.

In certain forms, the controller 838 may cause the generator 800 to provide audible or other sensory feedback for alerting the user that a power on or power off sequence has been initiated. Such an alert may be provided at the beginning of a power on or power off sequence and prior to the commencement of other processes associated with the sequence.

In certain forms, the isolated stage 802 may comprise an instrument interface circuit 840 to, for example, provide a communication interface between a control circuit of a surgical instrument (e.g., a control circuit comprising handpiece switches) and components of the non-isolated stage 804, such as, for example, the logic device 816, the DSP processor 822, and/or the UI processor 836. The instrument interface circuit 840 may exchange information with components of the non-isolated stage 804 via a communication link that maintains a suitable degree of electrical isolation between the isolated and non-isolated stages 802, 804, such as, for example, an IR-based communication link. Power may be supplied to the instrument interface circuit 840 using, for example, a low-dropout voltage regulator powered by an isolation transformer driven from the non-isolated stage 804.

In one form, the instrument interface circuit 840 may comprise a logic circuit 842 (e.g., logic circuit, programmable logic circuit, PGA, FPGA, PLD) in communication with a signal conditioning circuit 844. The signal conditioning circuit 844 may be configured to receive a periodic signal from the logic circuit 842 (e.g., a 2 kHz square wave) to generate a bipolar interrogation signal having an identical frequency. The interrogation signal may be generated, for example, using a bipolar current source fed by a differential amplifier. The interrogation signal may be communicated to a surgical instrument control circuit (e.g., by using a conductive pair in a cable that connects the generator 800 to the surgical instrument) and monitored to determine a state or configuration of the control circuit. The control circuit may comprise a number of switches, resistors, and/or diodes to modify one or more characteristics (e.g., amplitude, rectification) of the interrogation signal such that a state or configuration of the control circuit is uniquely discernable based on the one or more characteristics. In one form, for example, the signal conditioning circuit 844 may comprise an ADC circuit for generating samples of a voltage signal appearing across inputs of the control circuit resulting from passage of interrogation signal therethrough. The logic circuit 842 (or a component of the non-isolated stage 804) may then determine the state or configuration of the control circuit based on the ADC circuit samples.

In one form, the instrument interface circuit 840 may comprise a first data circuit interface 846 to enable information exchange between the logic circuit 842 (or other element of the instrument interface circuit 840) and a first data circuit disposed in or otherwise associated with a surgical instrument. In certain forms, for example, a first data circuit may be disposed in a cable integrally attached to a surgical instrument handpiece or in an adaptor for interfacing a specific surgical instrument type or model with the generator 800. The first data circuit may be implemented in any suitable manner and may communicate with the generator according to any suitable protocol, including, for example, as described herein with respect to the first data circuit. In certain forms, the first data circuit may comprise a non-volatile storage device, such as an EEPROM device. In certain forms, the first data circuit interface 846 may be implemented separately from the logic circuit 842 and comprise suitable circuitry (e.g., discrete logic devices, a processor) to enable communication between the logic circuit 842 and the first data circuit. In other forms, the first data circuit interface 846 may be integral with the logic circuit 842.

In certain forms, the first data circuit may store information pertaining to the particular surgical instrument with which it is associated. Such information may include, for example, a model number, a serial number, a number of operations in which the surgical instrument has been used, and/or any other type of information. This information may be read by the instrument interface circuit 840 (e.g., by the logic circuit 842), transferred to a component of the non-isolated stage 804 (e.g., to logic device 816, DSP processor 822, and/or UI processor 836) for presentation to a user via an output device and/or for controlling a function or operation of the generator 800. Additionally, any type of information may be communicated to the first data circuit for storage therein via the first data circuit interface 846 (e.g., using the logic circuit 842). Such information may comprise, for example, an updated number of operations in which the surgical instrument has been used and/or dates and/or times of its usage.

As discussed previously, a surgical instrument may be detachable from a handpiece (e.g., the multifunction surgical instrument may be detachable from the handpiece) to promote instrument interchangeability and/or disposability. In such cases, conventional generators may be limited in their ability to recognize particular instrument configurations being used and to optimize control and diagnostic processes accordingly. The addition of readable data circuits to surgical instruments to address this issue is problematic from a compatibility standpoint, however. For example, designing a surgical instrument to remain backwardly compatible with generators that lack the requisite data reading functionality may be impractical due to, for example, differing signal schemes, design complexity, and cost. Forms of instruments discussed herein address these concerns by using data circuits that may be implemented in existing surgical instruments economically and with minimal design changes to preserve compatibility of the surgical instruments with current generator platforms.

Additionally, forms of the generator 800 may enable communication with instrument-based data circuits. For example, the generator 800 may be configured to communicate with a second data circuit contained in an instrument (e.g., the multifunction surgical instrument). In some forms, the second data circuit may be implemented in a many similar to that of the first data circuit described herein. The instrument interface circuit 840 may comprise a second data circuit interface 848 to enable this communication. In one form, the second data circuit interface 848 may comprise a tri-state digital interface, although other interfaces may also be used. In certain forms, the second data circuit may generally be any circuit for transmitting and/or receiving data. In one form, for example, the second data circuit may store information pertaining to the particular surgical instrument with which it is associated. Such information may include, for example, a model number, a serial number, a number of operations in which the surgical instrument has been used, and/or any other type of information.

In some forms, the second data circuit may store information about the electrical and/or ultrasonic properties of an associated ultrasonic transducer, end effector, or ultrasonic drive system. For example, the first data circuit may indicate a burn-in frequency slope, as described herein. Additionally or alternatively, any type of information may be communicated to second data circuit for storage therein via the second data circuit interface 848 (e.g., using the logic circuit 842). Such information may comprise, for example, an updated number of operations in which the instrument has been used and/or dates and/or times of its usage. In certain forms, the second data circuit may transmit data acquired by one or more sensors (e.g., an instrument-based temperature sensor). In certain forms, the second data circuit may receive data from the generator 800 and provide an indication to a user (e.g., a light emitting diode indication or other visible indication) based on the received data.

In certain forms, the second data circuit and the second data circuit interface 848 may be configured such that communication between the logic circuit 842 and the second data circuit can be effected without the need to provide additional conductors for this purpose (e.g., dedicated conductors of a cable connecting a handpiece to the generator 800). In one form, for example, information may be communicated to and from the second data circuit using a one-wire bus communication scheme implemented on existing cabling, such as one of the conductors used transmit interrogation signals from the signal conditioning circuit 844 to a control circuit in a handpiece. In this way, design changes or modifications to the surgical instrument that might otherwise be necessary are minimized or reduced. Moreover, because different types of communications implemented over a common physical channel can be frequency-band separated, the presence of a second data circuit may be “invisible” to generators that do not have the requisite data reading functionality, thus enabling backward compatibility of the surgical instrument.

In certain forms, the isolated stage 802 may comprise at least one blocking capacitor 850-1 connected to the drive signal output 810b to prevent passage of DC current to a patient. A single blocking capacitor may be required to comply with medical regulations or standards, for example. While failure in single-capacitor designs is relatively uncommon, such failure may nonetheless have negative consequences. In one form, a second blocking capacitor 850-2 may be provided in series with the blocking capacitor 850-1, with current leakage from a point between the blocking capacitors 850-1, 850-2 being monitored by, for example, an ADC circuit 852 for sampling a voltage induced by leakage current. The samples may be received by the logic circuit 842, for example. Based changes in the leakage current (as indicated by the voltage samples), the generator 800 may determine when at least one of the blocking capacitors 850-1, 850-2 has failed, thus providing a benefit over single-capacitor designs having a single point of failure.

In certain forms, the non-isolated stage 804 may comprise a power supply 854 for delivering DC power at a suitable voltage and current. The power supply may comprise, for example, a 400 W power supply for delivering a 48 VDC system voltage. The power supply 854 may further comprise one or more DC/DC voltage converters 856 for receiving the output of the power supply to generate DC outputs at the voltages and currents required by the various components of the generator 800. As discussed above in connection with the controller 838, one or more of the DC/DC voltage converters 856 may receive an input from the controller 838 when activation of the “on/off” input device by a user is detected by the controller 838 to enable operation of, or wake, the DC/DC voltage converters 856.

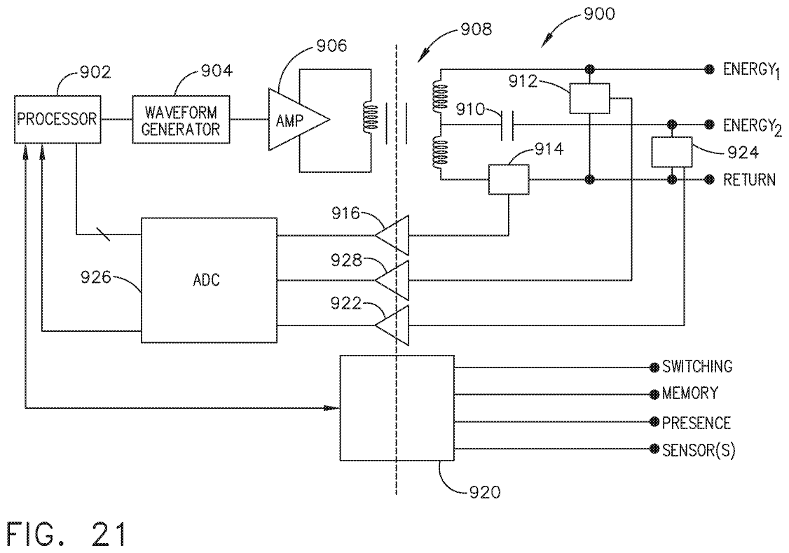

FIG. 21 illustrates an example of a generator 900, which is one form of the generator 800 (FIG. 21). The generator 900 is configured to deliver multiple energy modalities to a surgical instrument. The generator 900 provides RF and ultrasonic signals for delivering energy to a surgical instrument either independently or simultaneously. The RF and ultrasonic signals may be provided alone or in combination and may be provided simultaneously. As noted above, at least one generator output can deliver multiple energy modalities (e.g., ultrasonic, bipolar or monopolar RF, irreversible and/or reversible electroporation, and/or microwave energy, among others) through a single port, and these signals can be delivered separately or simultaneously to the end effector to treat tissue.

The generator 900 comprises a processor 902 coupled to a waveform generator 904. The processor 902 and waveform generator 904 are configured to generate a variety of signal waveforms based on information stored in a memory coupled to the processor 902, not shown for clarity of disclosure. The digital information associated with a waveform is provided to the waveform generator 904 which includes one or more DAC circuits to convert the digital input into an analog output. The analog output is fed to an amplifier 1106 for signal conditioning and amplification. The conditioned and amplified output of the amplifier 906 is coupled to a power transformer 908. The signals are coupled across the power transformer 908 to the secondary side, which is in the patient isolation side. A first signal of a first energy modality is provided to the surgical instrument between the terminals labeled ENERGY1 and RETURN. A second signal of a second energy modality is coupled across a capacitor 910 and is provided to the surgical instrument between the terminals labeled ENERGY2 and RETURN. It will be appreciated that more than two energy modalities may be output and thus the subscript “n” may be used to designate that up to n ENERGYn terminals may be provided, where n is a positive integer greater than 1. It also will be appreciated that up to “n” return paths RETURNn may be provided without departing from the scope of the present disclosure.

A first voltage sensing circuit 912 is coupled across the terminals labeled ENERGY1 and the RETURN path to measure the output voltage therebetween. A second voltage sensing circuit 924 is coupled across the terminals labeled ENERGY2 and the RETURN path to measure the output voltage therebetween. A current sensing circuit 914 is disposed in series with the RETURN leg of the secondary side of the power transformer 908 as shown to measure the output current for either energy modality. If different return paths are provided for each energy modality, then a separate current sensing circuit should be provided in each return leg. The outputs of the first and second voltage sensing circuits 912, 924 are provided to respective isolation transformers 916, 922 and the output of the current sensing circuit 914 is provided to another isolation transformer 918. The outputs of the isolation transformers 916, 928, 922 in the on the primary side of the power transformer 908 (non-patient isolated side) are provided to a one or more ADC circuit 926. The digitized output of the ADC circuit 926 is provided to the processor 902 for further processing and computation. The output voltages and output current feedback information can be employed to adjust the output voltage and current provided to the surgical instrument and to compute output impedance, among other parameters. Input/output communications between the processor 902 and patient isolated circuits is provided through an interface circuit 920. Sensors also may be in electrical communication with the processor 902 by way of the interface circuit 920.

In one aspect, the impedance may be determined by the processor 902 by dividing the output of either the first voltage sensing circuit 912 coupled across the terminals labeled ENERGY1/RETURN or the second voltage sensing circuit 924 coupled across the terminals labeled ENERGY2/RETURN by the output of the current sensing circuit 914 disposed in series with the RETURN leg of the secondary side of the power transformer 908. The outputs of the first and second voltage sensing circuits 912, 924 are provided to separate isolations transformers 916, 922 and the output of the current sensing circuit 914 is provided to another isolation transformer 916. The digitized voltage and current sensing measurements from the ADC circuit 926 are provided the processor 902 for computing impedance. As an example, the first energy modality ENERGY1 may be ultrasonic energy and the second energy modality ENERGY2 may be RF energy.

Nevertheless, in addition to ultrasonic and bipolar or monopolar RF energy modalities, other energy modalities include irreversible and/or reversible electroporation and/or microwave energy, among others. Also, although the example illustrated in FIG. 21 shows a single return path RETURN may be provided for two or more energy modalities, in other aspects, multiple return paths RETURNn may be provided for each energy modality ENERGYn. Thus, as described herein, the ultrasonic transducer impedance may be measured by dividing the output of the first voltage sensing circuit 912 by the current sensing circuit 914 and the tissue impedance may be measured by dividing the output of the second voltage sensing circuit 924 by the current sensing circuit 914.

As shown in FIG. 21, the generator 900 comprising at least one output port can include a power transformer 908 with a single output and with multiple taps to provide power in the form of one or more energy modalities, such as ultrasonic, bipolar or monopolar RF, irreversible and/or reversible electroporation, and/or microwave energy, among others, for example, to the end effector depending on the type of treatment of tissue being performed. For example, the generator 900 can deliver energy with higher voltage and lower current to drive an ultrasonic transducer, with lower voltage and higher current to drive RF electrodes for sealing tissue, or with a coagulation waveform for spot coagulation using either monopolar or bipolar RF electrosurgical electrodes. The output waveform from the generator 900 can be steered, switched, or filtered to provide the frequency to the end effector of the surgical instrument. The connection of an ultrasonic transducer to the generator 900 output would be preferably located between the output labeled ENERGY1 and RETURN as shown in FIG. 21. In one example, a connection of RF bipolar electrodes to the generator 900 output would be preferably located between the output labeled ENERGY2 and RETURN. In the case of monopolar output, the preferred connections would be active electrode (e.g., pencil or other probe) to the ENERGY2 output and a suitable return pad connected to the RETURN output.

Additional details are disclosed in U.S. Patent Application Publication No. 2017/0086914, titled TECHNIQUES FOR OPERATING GENERATOR FOR DIGITALLY GENERATING ELECTRICAL SIGNAL WAVEFORMS AND SURGICAL INSTRUMENTS, which published on Mar. 30, 2017, which is herein incorporated by reference in its entirety.

Robotic surgical systems can be used in minimally invasive medical procedures. During such medical procedures, a patient can be placed on a platform adjacent to a robotic surgical system, and a surgeon can be positioned at a console that is remote from the platform and/or from the robot. For example, the surgeon can be positioned outside the sterile field that surrounds the surgical site. The surgeon provides input to a user interface via an input device at the console to manipulate a surgical tool coupled to an arm of the robotic system. The input device can be a mechanical input devices such as control handles or joysticks, for example, or contactless input devices such as optical gesture sensors, for example.

The robotic surgical system can include a robot tower supporting one or more robotic arms. At least one surgical tool (e.g. an end effector and/or endoscope) can be mounted to the robotic arm. The surgical tool(s) can be configured to articulate relative to the respective robotic arm via an articulating wrist assembly and/or to translate relative to the robotic arm via a linear slide mechanism, for example. During the surgical procedure, the surgical tool can be inserted into a small incision in a patient via a cannula or trocar, for example, or into a natural orifice of the patient to position the distal end of the surgical tool at the surgical site within the body of the patient. Additionally or alternatively, the robotic surgical system can be employed in an open surgical procedure in certain instances.

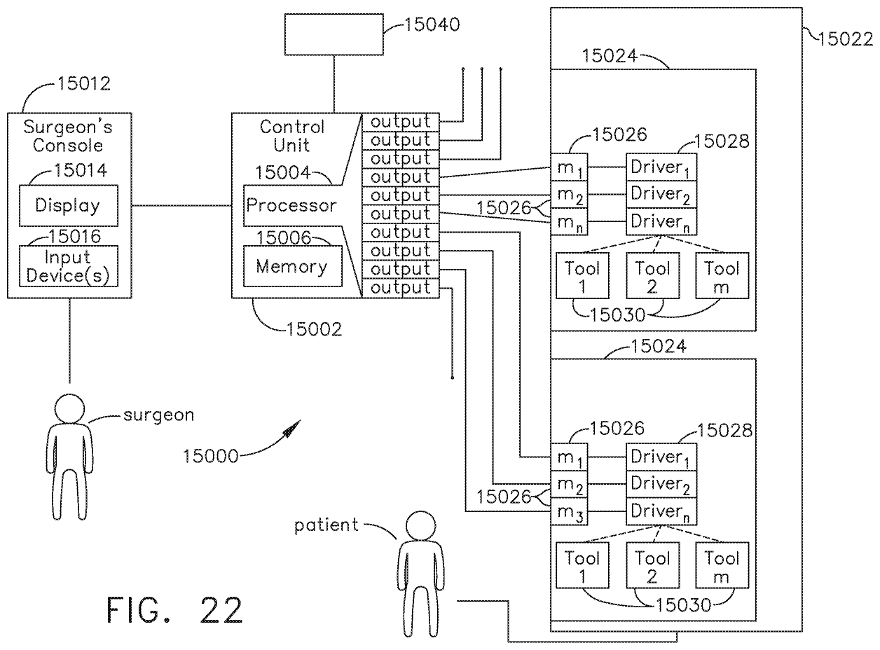

A schematic of a robotic surgical system 15000 is depicted in FIG. 22. The robotic surgical system 15000 includes a central control unit 15002, a surgeon's console 15012, a robot 15022 including one or more robotic arms 15024, and a primary display 15040 operably coupled to the control unit 15002. The surgeon's console 15012 includes a display 15014 and at least one manual input device 15016 (e.g., switches, buttons, touch screens, joysticks, gimbals, etc.) that allow the surgeon to telemanipulate the robotic arms 15024 of the robot 15022. The reader will appreciate that additional and alternative input devices can be employed.

The central control unit 15002 includes a processor 15004 operably coupled to a memory 15006. The processor 15004 includes a plurality of inputs and outputs for interfacing with the components of the robotic surgical system 15000. The processor 15004 can be configured to receive input signals and/or generate output signals to control one or more of the various components (e.g., one or more motors, sensors, and/or displays) of the robotic surgical system 15000. The output signals can include, and/or can be based upon, algorithmic instructions which may be pre-programmed and/or input by the surgeon or another clinician. The processor 15004 can be configured to accept a plurality of inputs from a user, such as the surgeon at the console 15012, and/or may interface with a remote system. The memory 15006 can be directly and/or indirectly coupled to the processor 15004 to store instructions and/or databases.

The robot 15022 includes one or more robotic arms 15024. Each robotic arm 15024 includes one or more motors 15026 and each motor 15026 is coupled to one or more motor drivers 15028. For example, the motors 15026, which can be assigned to different drivers and/or mechanisms, can be housed in a carriage assembly or housing. In certain instances, a transmission intermediate a motor 15026 and one or more drivers 15028 can permit coupling and decoupling of the motor 15026 to one or more drivers 15028. The drivers 15028 can be configured to implement one or more surgical functions. For example, one or more drivers 15028 can be tasked with moving a robotic arm 15024 by rotating the robotic arm 15024 and/or a linkage and/or joint thereof. Additionally, one or more drivers 15028 can be coupled to a surgical tool 15030 and can implement articulating, rotating, clamping, sealing, stapling, energizing, firing, cutting, and/or opening, for example. In certain instances, the surgical tools 15030 can be interchangeable and/or replaceable. Examples of robotic surgical systems and surgical tools are further described herein.

The reader will readily appreciate that the computer-implemented interactive surgical system 100 (FIG. 1) and the computer-implemented interactive surgical system 200 (FIG. 9) can incorporate the robotic surgical system 15000. Additionally or alternatively, the robotic surgical system 15000 can include various features and/or components of the computer-implemented interactive surgical systems 100 and 200.

In one exemplification, the robotic surgical system 15000 can encompass the robotic system 110 (FIG. 2), which includes the surgeon's console 118, the surgical robot 120, and the robotic hub 122. Additionally or alternatively, the robotic surgical system 15000 can communicate with another hub, such as the surgical hub 106, for example. In one instance, the robotic surgical system 15000 can be incorporated into a surgical system, such as the computer-implemented interactive surgical system 100 (FIG. 1) or the computer-implemented interactive surgical system 200 (FIG. 9), for example. In such instances, the robotic surgical system 15000 may interact with the cloud 104 or the cloud 204, respectively, and the surgical hub 106 or the surgical hub 206, respectively. In certain instances, a robotic hub or a surgical hub can include the central control unit 15002 and/or the central control unit 15002 can communicate with a cloud. In other instances, a surgical hub can embody a discrete unit that is separate from the central control unit 15002 and which can communicate with the central control unit 15002.

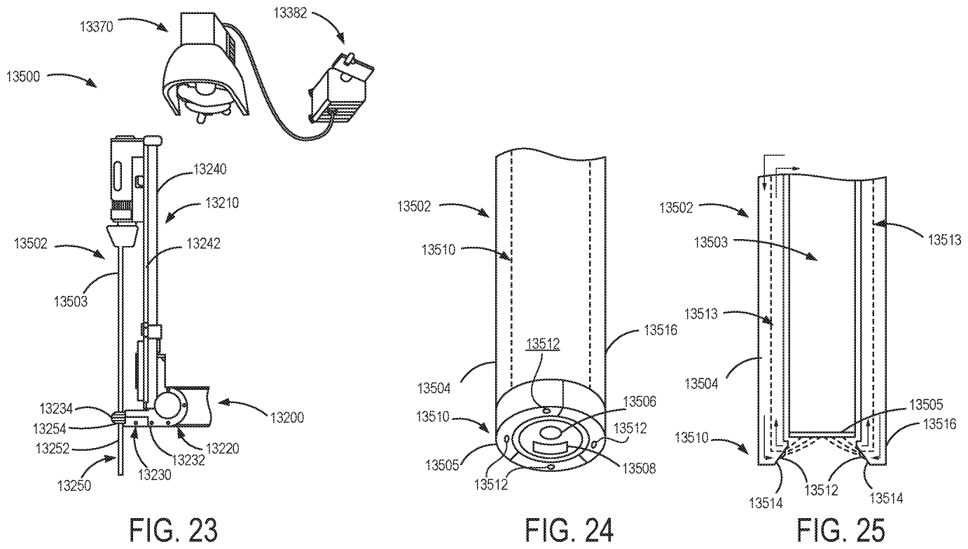

Referring primarily to FIGS. 23-25, a surgical visualization system 13500 includes a surgical visualization assembly 13502 coupled to a robotic arm 13200, which is similar in many respects to the robotic arms 13002, 13003 (FIG. 4). The robotic arm 13200 is part of a surgical robotic system 13360 (FIG. 6) that includes a remote command console 13370 (FIG. 6) and a surgical hub 13382 (FIG. 6). Other surgical robotic systems suitable for use with the visualization assembly 13502 include the surgical robotic systems 13000 (FIG. 4), 13400 (FIG. 5). In one example, the surgical visualization assembly 13502 is integrated with the robotic arm 13200. In another example, the surgical visualization assembly 13502 is releasably coupled to the robotic arm 13200. In various examples, the visualization assembly 13502 can be incorporated into a hand-held surgical visualization system for direct user manipulation in a laparoscopic or open surgery, for example.

Referring to FIG. 23, a side view is provided of a robotic arm 13200 including a mounting assembly 13210 for securing surgical tools thereto such as, for example, the visualization assembly 13502. The robotic arm 13200 is made up of three members connected via joints. The mounting assembly 13210 is coupled to a distal end 13220 of the arm 13200 and includes a mounting device 13230 and a longitudinally-extending support 13240. The mounting device 13230 is made up of a housing 13232 which supports a clamping and release assembly 13234 and is configured to selectively secure a variety of surgical tools therein to thereby secure a surgical tool to the robotic arm 13200. Although the mounting device 13230 may be adapted to receive a variety of surgical tools, the mounting device 13230 receives a trocar 13250. The trocar 13250 is releasably secured within the mounting device 13230 through a transition between an open configuration and a closed configuration of the clamping assembly 13234. The trocar 13250 includes a cannula 13252 configured to provide a pathway to a surgical site within the patient and has an access port 13254 for receiving a portion of the visualization assembly 13502.

The longitudinally-extending support 13240 extends substantially perpendicularly relative to the housing 13232 of the mounting device 13230 and supports a vertical rail 13242. The vertical rail 13242 is coupled to the support 13240 and extends along a length of the support 13240. The vertical rail 13242 is configured such that the visualization assembly 13502 may be slidably coupled thereto and aligned with the trocar 13250. In particular, a shaft 262 of the of the imaging device 13503 is substantially aligned with the trocar 13250 so that it can be inserted into or removed from the access port 13254 of the trocar 13250.

In the example illustrated in FIGS. 24 and 25, the surgical visualization assembly 13502 includes an imaging device 13503 and an outer housing 13504 in the form of a tubular member partially encapsulating the imaging device 13503. Specifically, a distal end 13505 of the imaging device 13503, which includes a visualization lens 13506 and a light source 13508, is exposed. In other examples, the distal end 13505 of the imaging device 13503 can be fully encapsulated by the outer housing 13504. The outer housing 13504 may include a transparent lens disposed in front of the distal end 13505 of the imaging device 13503 to protect the visualization lens 13506 and/or the light source 13508 from direct exposure to body fluids. For the purposes of the present disclosure a transparent lens positioned in front of the visualization lens 13506 is considered part of the visualization lens 13506.

Further to the above, the imaging device 13503 is similar in many respects to other imaging devices described in the present disclosure such as, for example, the imaging device 124. Like the imaging device 124, the imaging device 13503 is configured for use in a minimally invasive procedure. In one aspect, the imaging device 13503 employs multi-spectrum monitoring to discriminate topography and underlying structures. The use of multi-spectral imaging is described in greater detail under the heading “Advanced Imaging Acquisition Module” in U.S. Provisional Patent Application Ser. No. 62/611,341, titled INTERACTIVE SURGICAL PLATFORM, filed Dec. 28, 2017, the disclosure of which is herein incorporated by reference in its entirety.

Referring still to FIGS. 24 and 25, the outer housing 13504 supports components of a lens cleaning system 13510 that includes fluid ports 13512 in the form of nozzles that are disposed at chamfered edges 13514 of a distal portion 13516 of the outer housing 13504 and are directed toward the distal end 13505 of the imaging device 13503. The fluid ports 13512 are connected to one or more fluid tubes 13513, and are configured to clean the distal end 13505 by ejecting fluids at and/or suctioning fluids in near proximity to the distal end 13505 to remove biological material, other debris, moisture/fog, contaminants, and/or any other materials accumulating onto the distal end 13505 against the visualization lens 13506. The fluids utilized by the lens cleaning system 13510 may include various suitable gases such as, for example carbon dioxide and/or liquids such as, for example saline.

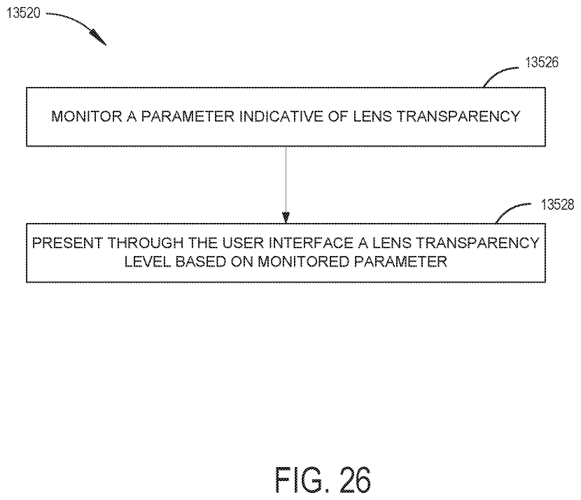

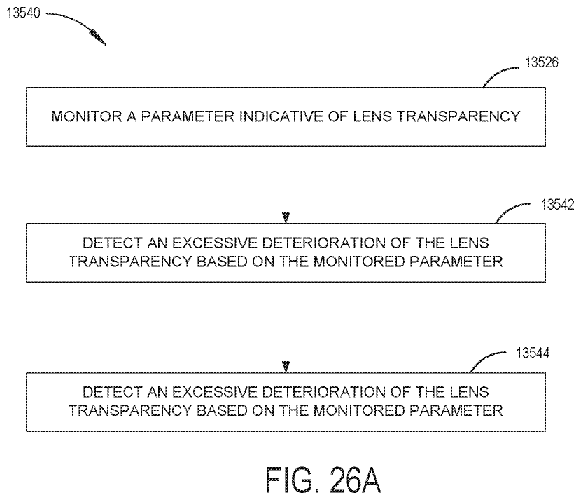

FIG. 26 is a logic flow diagram of a process 13520 depicting a control program or a logic configuration for determining whether a visualization lens 13506 of a surgical visualization system 13500 needs cleaning and/or reporting the same. In various instances, the process 13520 includes monitoring 13526 a parameter indicative of lens transparency or lens occlusion. The process 13520 further includes presenting 13528 through a user interface 13522 of the surgical visualization system 13500 a lens transparency level or lens occlusion level based on the monitored parameter.

FIG. 26A is a is a logic flow diagram of a process 13540 depicting a control program or a logic configuration for determining whether a visualization lens 13506 of a surgical visualization system 13500 needs cleaning and triggering the cleaning. In various instances, the process 13540 includes monitoring 13526 a parameter indicative of lens transparency or lens occlusion of the visualization lens 13506. The process 13520 further includes detecting 13542 an excessive deterioration of lens transparency based on the monitored parameter, and automatically activating 13544, or triggering activation, of a lens cleaning system 13510 to remedy the excessive deterioration of the lens transparency.

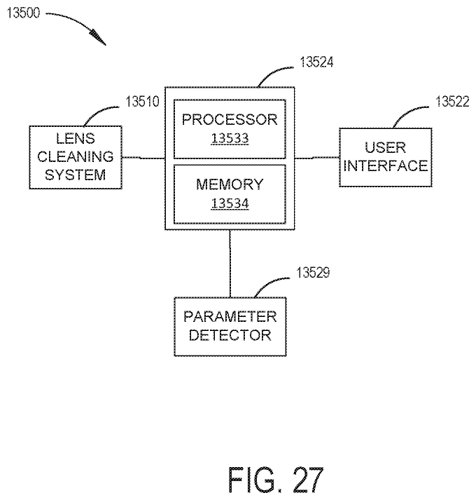

In various examples, as illustrated in FIG. 27, the surgical visualization system 13500 includes a control circuit 13524 configured to perform the processes 13520, 13540. In the example of FIG. 27, one or both of the processes 13520, 13540 can be embodied as a set of computer-executable instructions stored in a memory 13534 that, when executed by a processor 13533, cause the processor 13533 to perform the processes 13520, 13540. In other examples, the processes 13520, 13540 can be performed by other control circuits such as, for example, the control circuits 500 (FIG. 13), 510 (FIG. 14), 520 (FIG. 15). Further, although the processes 13520, 13540 is described as being executed by a control circuit 13524, this is merely for brevity, and it should be understood that the depicted processes 13520, 13540 can be executed by circuitry that can include a variety of hardware and/or software components and may be located in or associated with various systems integral or connected to a robotic surgical system.

Further to the above, the control circuit 13524 is coupled to a parameter detector 13529 which can be configured to measure values of a parameter indicative of lens transparency or lens occlusion of the visualization lens 13506, in accordance with the processes 13520, 13540. For the purposes of the present disclosure, the terms lens transparency and lens occlusion, although possessing opposite meanings, represent a degree of visibility through the visualization lens 13506. Greater lens transparency correlates to a reduction in lens occlusion.

In addition, the control circuit 13524 is coupled to a user interface 13522. In one example, the user interface 13522 can be at a remote command console 13370 (FIG. 6). In another example, the user interface 13522 can be in the form of an interactive secondary display that is similar in many respects to the interactive secondary displays 13362, 13364 (FIG. 7). Furthermore, the control circuit 13524 is coupled to a lens cleaning system 13510 that may include, for example, fluid pumps for ejecting and/or suctioning fluids at, or near, the distal end 13505 of the imaging device 13503.

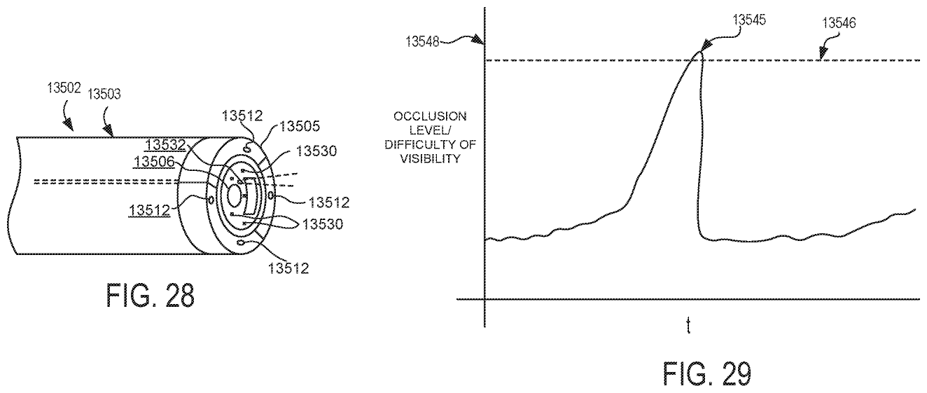

In one example, the parameter is time. In other words, the control circuit 13524 is configured to trigger activation of the lens cleaning system 13510 at predetermined time intervals. Additionally, or alternatively, the parameter detector 13529 may include one or more capacitive sensors 13530 disposed at, or near, the distal end 13505 of the imaging device 13503. In one example, the parameter detector 13529 includes a series of capacitive sensors 13530 disposed at, or near, the distal end 13505 around the visualization lens 13506, as illustrated in FIG. 28. The capacitive sensors 13530 detect biological materials and/or other debris accumulation at, or near, the visualization lens 130506. The measured values of the capacitive sensors 13530 represent lens occlusion or transparency levels of the visualization lens 13506. A look-up table or database can be accessed by the control circuit to determine the lens occlusion or transparency levels based on the measured values of the capacitive sensors 13530.

In one example, the memory 13534 of the control circuit 13524 may store an algorithm, an equation, or a look-up table for determining correlations between measurements of one or more of the capacitive sensors 13530 and the lens occlusion or transparency levels of the visualization lens 13506. In addition, a processor 13533 of the control circuit 13524 may employ such algorithm, equation, and/or look-up table to determine the lens occlusion or transparency levels based on the measurements of the capacitive sensors 13530. In certain instances, each of the capacitive sensors 13530 can be assigned to a specific area or portion of the distal end 13505 of the imaging device 13503 to detect biological materials and/or other debris accumulation at, or near, such area or portion. In such instances, different lens occlusion or transparency levels can be ascertained for different areas or portions of the distal end 13505. Alternatively, or additionally, the measurements of some or all of the capacitive sensors 13530 can be aggregated to derive a common lens occlusion or transparency level at the distal end 13505.

Additionally, or alternatively, the parameter detector 13529 may include one or more optical sensors 13532 disposed at, or near, the distal end 13505 of the imaging device 13503. In one example, the parameter detector 13529 includes a series of optical sensors 13532 disposed at, or near, the distal end 13505 around the visualization lens 13506. The optical sensors 13532 detect biological materials and/or other debris accumulation at, or near, the visualization lens 130506 by measuring changes in light diffusion caused by the accumulation. The measured values of the optical sensors 13532 represent lens occlusion or transparency levels of the visualization lens 13506. A look-up table or database can be accessed by the control circuit to determine the lens occlusion or transparency levels based on the measured values of the optical sensors 13532.

In one example, the memory 13534 of the control circuit 13524 may store an algorithm, an equation, or a look-up table for determining correlations between measurements of one or more of the optical sensors 13532 and the lens occlusion or transparency levels of the visualization lens 13506. In addition, a processor 13533 of the control circuit 13524 may employ such algorithm, equation, and/or look-up table to determine the lens occlusion or transparency levels based on the measurements of the optical sensors 13532. In certain instances, each of the optical sensors 13532 can be assigned to a specific area or portion of the distal end 13505 of the imaging device 13503 to detect biological materials and/or other debris accumulation at, or near, such area or portion. In such instances, different lens occlusion or transparency levels can be ascertained for different areas or portions of the distal end 13505. Alternatively, or additionally, the measurements of some or all of the optical sensors 13532 can be aggregated to derive a common lens occlusion or transparency level at the distal end 13505.

In at least one example, a secondary light activator is passed through the face of the visualization lens 13506 and is proportionate to the lens occlusion. In at least one example, an infrared light (“IR”) can be passed laterally through a transparent member in front of the visualization lens 13506 and, then, light diffusion in-between imaging passes can be detected. An increase in light diffusion would indicate accumulation of biological materials and/or other debris or contaminants against the visualization lens 13506. The control circuit 13524 can be configured to trigger activation of the lens cleaning system 13510 when the detected light diffusion is greater than or equal to a predetermined threshold that can be stored in the memory 13534, for example.

In various examples, the measurements of the parameter detector 13529 can be compared to a predetermined threshold to assess whether the lens cleaning system 13510 should be activated. In at least one example, as illustrated in FIG. 29, the predetermined threshold is a visibility threshold 13546, and the lens cleaning system 13510 is activated by the control circuit 13524 when the lens occlusion level 13548, as derived from the measurements of the parameter detector 13529, passes 13545 the visibility threshold 13546. Further, the cleaning system 13510 can be automatically deactivated by the control circuit 13524 when the lens occlusion level 13548 falls below the predetermined threshold 13546.

In various examples, the control circuit 13524 may utilize the imaging module 138 (FIG. 3) to determine when to trigger the activation of the cleaning system 13510. The imaging module 138 can be utilized to analyze and/or compare frames captured by the imaging device 13503 looking for either known makers (on instruments) or distinguishable objects within the field of view of the visualization lens 13506 to identify irregular distortions or blurriness beyond accepted predetermined thresholds. Accordingly, the control circuit 13524 can trigger the activation of the cleaning system 13510 based on input from the imaging module 138 indicative of identification of irregular distortions from one or more frames capture by the imaging device 13503 through the visualization lens 13506. If the irregular distortions remain after cleaning is completed, the control circuit 13524 may delay re-triggering of the activation of the lens cleaning system 13510 a predefined amount of time or ignore the irregular distortions in future determinations.

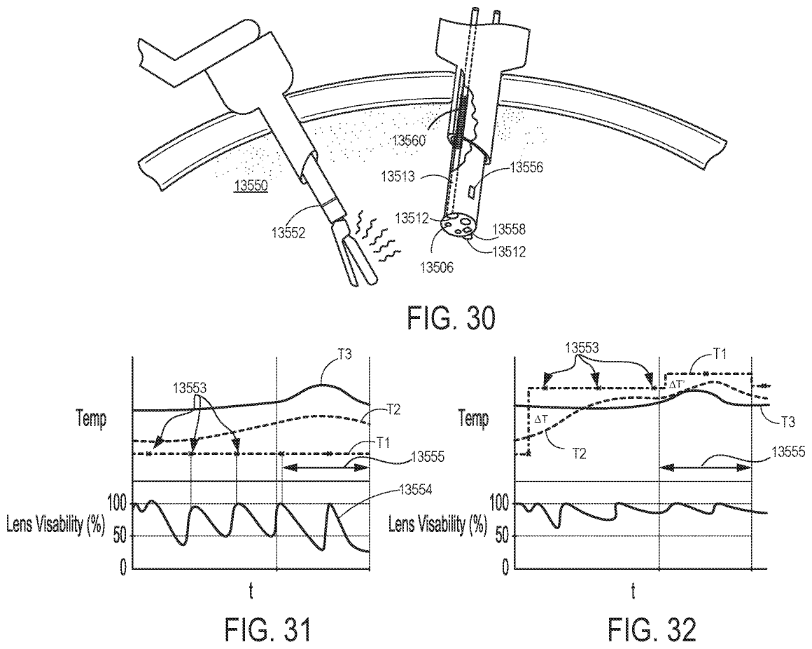

Referring primarily to FIGS. 30-32, in addition to biological material, debris, and/or contaminants, lens fogging is another factor that affects the lens occlusion and transparency levels. Lens fogging occurs when the temperature of a lens becomes lower than its surrounding environment. As illustrated in FIG. 30, a visualization lens 13506 is generally used inside a body cavity 13550 of a patient such as, for example, the abdominal cavity where the temperature is T3. The temperature of the lens outside a patient's body cavity 13550 is room temperature, which is less than the temperature T3. Accordingly, the visualization lens 13506 may fog during, or directly after, introduction into the body cavity 13550.

As illustrated in FIG. 31, lens fogging may also occur after lens cleaning is performed by the lens cleaning system 13510 if the cleaning fluid ejected by the lens cleaning system 13510 is at a temperature T1 below the temperature T3 of the body cavity 13550. The bottom graph of FIG. 31 illustrates how lens visibility 13554 slowly and repeatedly decreases after application 13553 of a cleaning cycle by the lens cleaning system 13510 with a cleaning fluid at the temperature T1 due to repeated fogging of the visualization lens 13506. Lens fogging continues to occur because the cleaning fluid, at temperature T1, maintains a temperature T2 of the visualization lens 13506 below the temperature T3 of the body cavity 13550. Said another way, the cleaning fluid cools the visualization lens 13506 causing lens fogging that, in turn, causes the control circuit 13524 to trigger additional activations of the lens cleaning system 13510. In other instances, lens fogging may occur because the temperature T3 of the body cavity 13550 increases due to external factors. As illustrated in FIG. 30, lens fogging may occur during a surgical procedure due to activation 13555 of an electrosurgical surgical instrument 13552 inside the body cavity 13550, which raises the temperature T3 of the body cavity 13550, as illustrated in FIG. 31.

Referring again to FIG. 27, in various aspects, the parameter detector 13529 may monitor the temperature of the visualization lens 13506, the temperature of the body cavity 13550, and/or the temperature of the cleaning fluid to track lens transparency or lens occlusion levels caused by lens fogging changes from the tracked temperatures. Furthermore, the control circuit 13524 may activate the lens cleaning system 13510 to improve lens transparency levels or reduce lens occlusion levels if it is determined, based on the measurements of the temperature of the visualization lens 13506, the temperature of the body cavity 13550, and/or the temperature of the cleaning fluid, that lens fogging has reached or exceeded a predetermined threshold.

Referring to FIG. 30, in various aspects, a visualization assembly 13502 includes one or more temperature sensors 13556 for measuring the temperature T3 of the body cavity 13550. The temperature sensors 13556 are disposed on a distal portion of the visualization assembly 13502 that is positioned within the body cavity 13550 during a surgical procedure. In other examples, the temperature sensors 13556 can be deployed in any suitable location within the body cavity 13550. The surgical visualization assembly 13502 further includes one or more temperature sensors 13558 form measuring the temperature T2 of the visualization lens 13506. The temperature sensors 13558 are disposed at the distal end 13505 near the visualization lens 13506. The surgical visualization assembly 13502 further includes one or more heating elements 13560 configured to adjust the temperature T1 of the cleaning fluid of the lens cleaning system.

In various aspects, the control circuit 13524 may control the temperature T1 of the cleaning fluid to a desired temperature through the heating elements 13560 in order to avoid, or at least reduce, lens fogging. As illustrated in the top graph of FIG. 32, the temperature T1 of the cleaning fluid is raised above the temperature T3 of the body cavity 13550 by an amount (AT) sufficient to maintain the temperature T2 of the visualization lens 13506 above, or at least at, the temperature T3 of the body cavity 13550. The result, as illustrated in bottom graph of FIG. 32, is a reduction in visibility 13554 fluctuation due to lens fogging, as evident from comparing the bottom graphs of FIGS. 31 and 32.

In various aspects, the control circuit 13524 can predict instances of fog occurrences based on the readings of the temperature sensors 13556, 13558, and adjust the temperature T1 of the cleaning fluid, the mount of cleaning fluid applied to the visualization lens 13506, and/or the frequency of cleaning fluid application to the visualization lens 13506 to avoid, or at least reduce, lens fogging. For example, as illustrated in FIGS. 30 and 32, activation of the electrosurgical instrument 13552 may increase the temperature T3 of the body cavity 13550. The control circuit 13524 may receive input from the temperature sensors 13556 indicative of the increase in the temperature T3. In response, the control circuit 13524 may cause the heating elements 13560 to be activated to raise the temperature T2 of the cleaning fluid an amount (AT′) and/or cause the lens cleaning system 13510 to increase the amount and/or frequency of application of the heated cleaning fluid to the visualization lens 13506 to maintain the temperature T2 above, or at least at, the increased temperature T3 of the body cavity 13550.

In various aspects, the fluid ports 13512 can be adjusted to control cleaning fluid direction and flow speed. In one example, the control circuit 13524 can be coupled to one or more motors that can move the fluid ports to adjust a flow direction of the cleaning fluid. The fluid ports 13512 may include adjustable openings to control the speed of flow. Additionally, or alternatively, the control circuit 13524 may adjust the flow speed of the cleaning fluid by adjusting power delivered to fluid pumps of the lens cleaning system 13510. The control circuit 13524 can adjust the control cleaning fluid direction and flow speed to effect removal or disposition of biological materials and/or other debris toward a portion of the abdomen which is not in use or toward predefined locations for collection or controlled re-introduction into the body. In various aspects, the control circuit 13524 is configured to adjust the flow speed of the cleaning fluid based on input from the imaging module 138 indicative of the type and/or size of the debris to be removed.

In various instances, automatic control of the activation of the lens cleaning system 13510 is further subject to a predetermined waiting period between consecutive activations. In such instances, the control circuit 13524 is prevented from triggering another lens cleaning system 13510 activation until the predetermined time period has passed.

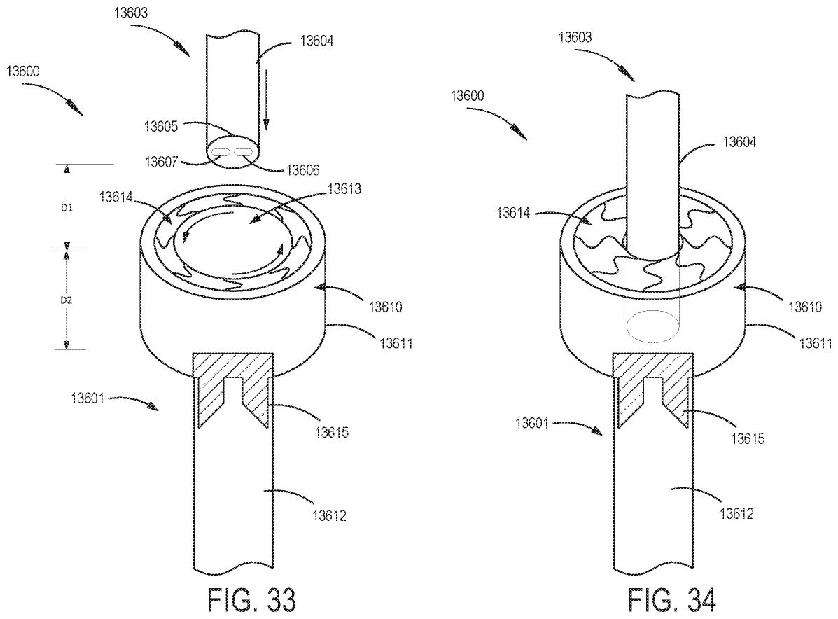

Referring to FIGS. 33 and 34, a visualization system 13600 includes an insertion port or trocar 13601, which is similar in many respects to the trocar 13250, and an imaging device 13603 insertable into a body cavity through the trocar 13601. The imaging device 13603 is similar in many respects to the imaging device 124 (FIG. 2). In various aspects, the visualization system 13600 is coupled to a robotic arm 13200. The robotic arm 13200 is part of a surgical robotic system 13360 (FIG. 6) that includes a remote command console 13370 (FIG. 6) and a surgical hub 13382 (FIG. 6). Other surgical robotic systems suitable for use with the visualization system 13600 include the surgical robotic systems 13000 (FIG. 4), 13400 (FIG. 5). In one example, the visualization system 13600 is integrated with the robotic arm 13200. In another example, the surgical visualization system 13600 is releasably coupled to the robotic arm 13200. In various examples, the visualization system 13600 can be incorporated into a hand-held surgical visualization system for direct user manipulation in a laparoscopic or open surgery, for example.

The trocar 13601 includes a seal assembly 13610 including an outer housing 13611. A tubular member 1612 extends distally from the outer housing 13611 and cooperates with the seal assembly 13610 to define a longitudinal opening 13613. The imaging device 13603 includes a shaft 13604 that has a distal end 13605 including a visualization lens 13606 and one or more light sources 13607. During a surgical procedure, the trocar 13601 is inserted through a body wall into a body cavity. The shaft 13604 is then inserted through the longitudinal opening of the trocar 13601 to introduce the distal end 13605 into the body cavity. As the surgical procedure progresses, biological material and/or other debris may accumulate on the visualization lens 13606 necessitating removal of imaging device 13603 from the trocar to clean the visualization lens 13606. In a typical trocar, reinsertion of a cleaned imaging device 13603 through the trocar may cause biological material and/or other debris left behind along the longitudinal opening of the trocar during removal of the imaging device 13603 for cleaning to be redeposited onto the visualization lens 13606.

To eliminate, or at least reduce, the redepositing of biological material and/or other debris onto the visualization lens 13606 during reinsertion of the imaging device 13603 through the trocar 13601, the seal assembly 13610 is automatically transitioned from a closed configuration (FIG. 34) to an open configuration (FIG. 33) to accommodate insertion of the imaging device 13603 into the longitudinal opening 13613. The seal assembly 13610 returns to the closed configuration after insertion of shaft 13604 through the seal assembly 13610, as illustrated in FIG. 34.

Referring still to FIGS. 33 and 34, the seal assembly 13610 includes an iris seal 13614 configured to constrict around the shaft 13604 of the imaging device 13603 in the closed configuration. In at least one example, the iris seal 13614 includes leaf members that rotate from a first relative position, substantially open, to a second relative position, substantially closed. Alternatively, the iris seal 13614 may be comprised of one or more elastic, flexible, and/or or shape changing elements that can be expanded, in the closed configuration, and retracted in the open configuration. In at least one example, the expandable elements may include a shape memory element such as, for example, Nitinol. In various aspects, an actuation mechanism for transitioning the iris seal 13614 between the open configuration and the closed configuration may include a motor, one or more sensors, and a control circuit for determining when to transition the iris seal 13614 between the open configuration and the closed configuration based on input signals received from the one or more sensors.

In various aspects, the control circuit is configured to transition the iris seal 13614 between the open configuration and the closed configuration according to the position of the trocar 13601 with respect to the imaging device 13603 and/or with respect to one or more components of the robotic arm 13200, for example. In various instances, the trocar 13601 and the imaging device 13603 are coupled to the robotic arm 13200. In such instances, the robotic arm 13200 causes the imaging device 13603 to be moved toward the trocar 13601 such that the shaft 13604 of the imaging device 13603 is inserted into the longitudinal opening of the trocar 13601. The distance between the distal end 13605 of the imaging device 13603 and the iris seal 13614 can be tracked by the surgical robotic system 13360, for example, by tracking the movement of the imaging device 13603 by the robotic arm 13200 and knowing the starting distance between the distal end 13605 of the imaging device 13603 and the iris seal 13614. As illustrated in FIG. 33, the iris seal 13614 is automatically opened when the distance between the iris seal 13614 and distal end 13605 is less than or equal to a predetermined distance D1. Furthermore, the iris seal 13614 is automatically closed or constricted around the shaft 13604 of the imaging device 13603 when the distal end 13605 has moved through the iris seal 13614 a distance greater than or equal to a predetermined distance D2, for example. This mechanism ensures that the biological material and/or other debris will not be repositioned on the cleaned visualization lens 13606.

In various instances, the above-described mechanism for opening and closing the iris seal 13614 can be similarly adopted with respect to other sealing features of the trocar 13601 such as, for example, an internal duckbill 13615. To prevent, or at least reduce, fluid insufflation loss during the above-described re-insertion process, the seals of a trocar 13601 can be opened and closed sequentially. For example, the iris seal 13614 can be opened then closed or constricted around the shaft 13604 before the distal end 13605 reaches a more distal seal such as, for example, the duckbill 13615. After closing the iris seal 13614, a more distal seal such as, for example, the duckbill 13615 is opened to allow passage of the distal end 13605. The duckbill 13615 is then constricted around the shaft 13604.

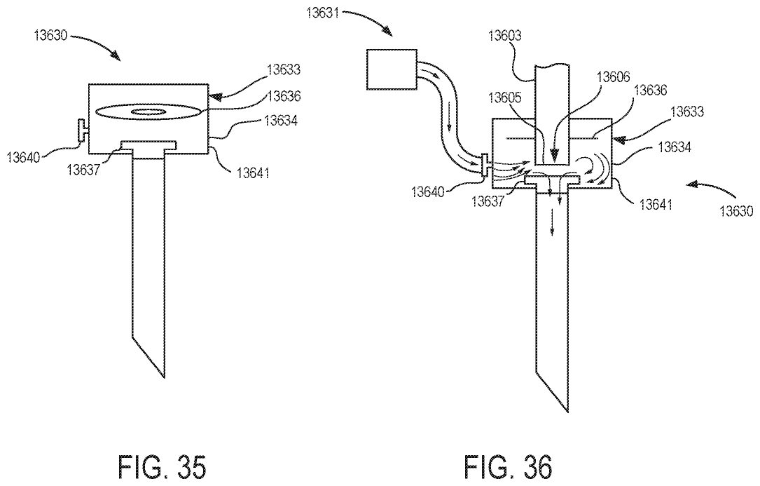

Referring to FIGS. 35 and 36, a trocar 13630 includes an integrated lens cleaning system 13631 configured to clean a visualization lens 13606 with fully removing the imaging device 13603 from the trocar 13630. The trocar 13630 includes a seal assembly 13633 that defines a cleaning chamber 13634 for removing biological material and/or other debris from the distal end 13605 of the imaging device 13603. The cleaning chamber 13634 defines an empty space between a proximal seal such as, for example, an iris seal 13636 and a distal seal 13637 such as, for example, a duckbill seal, the empty space being dimensioned to receiving the distal end 13605 of the imaging device 13603. An inlet port 13640 is defined in an outer housing 13641 of the seal assembly 13633. The inlet port 13640 passes flushing fluid from a lens cleaning system 13631 into the empty space of the cleaning chamber 13634. The flushing fluid removes biological material and/or other debris from the distal end 13605 of the imaging device 13603, and exits the cleaning chamber 13634 through the distal seal 13637, for example. Alternatively, the an outlet port can also be defined in the outer housing 13641 for facilitated removal of the flushing fluid and biological material and/or debris from the cleaning chamber 13634 to a collection chamber integrated with, or separate from, the cleaning system 13631.

In various aspects, the position of the imaging device 13603 with respect to a trocar 13630 that is connected to the robotic arm 136120 is controlled and monitored by the robotic surgical system 13360 (FIG. 6). Accordingly, the robotic surgical system 13360 can detect the presence of the distal end 13605 of the imaging device 13603 is in the empty space of the cleaning chamber 13634. Alternatively, or additionally, one or more sensors and/or integrated encoders can be positioned at, or near, the distal end 13605 to detect the presence of the distal end 13605 in the empty space of the cleaning chamber 13634. A control circuit such as, for example, the control circuit 500 can be configured to receive input indicating that the distal end 13605 is in the empty space of the cleaning chamber 13634. In response, the control circuit 500 automatically activates the lens cleaning system 13631 to cause the flushing fluid to remove biological material and/or other debris from the visualization lens 13606, for example. In other aspects, the control circuit 500 may signal a user through a user interface that the imaging device 13603 is ready for cleaning.

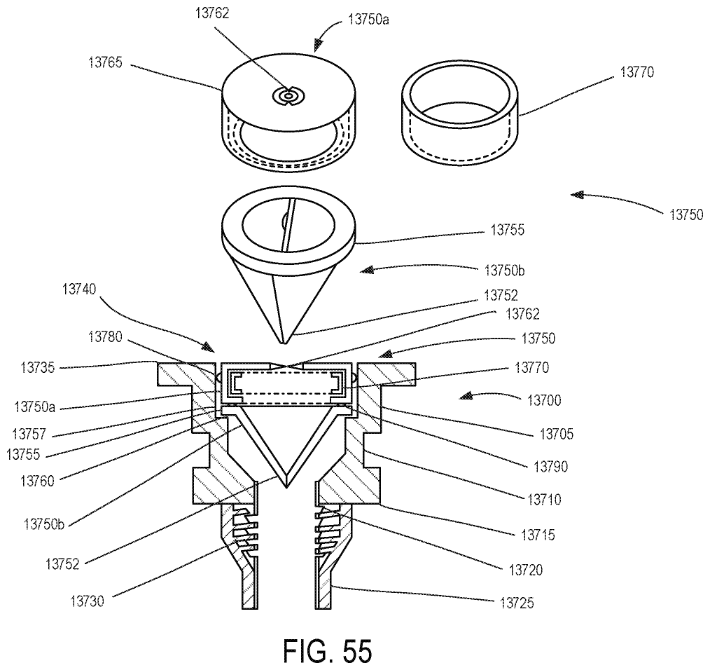

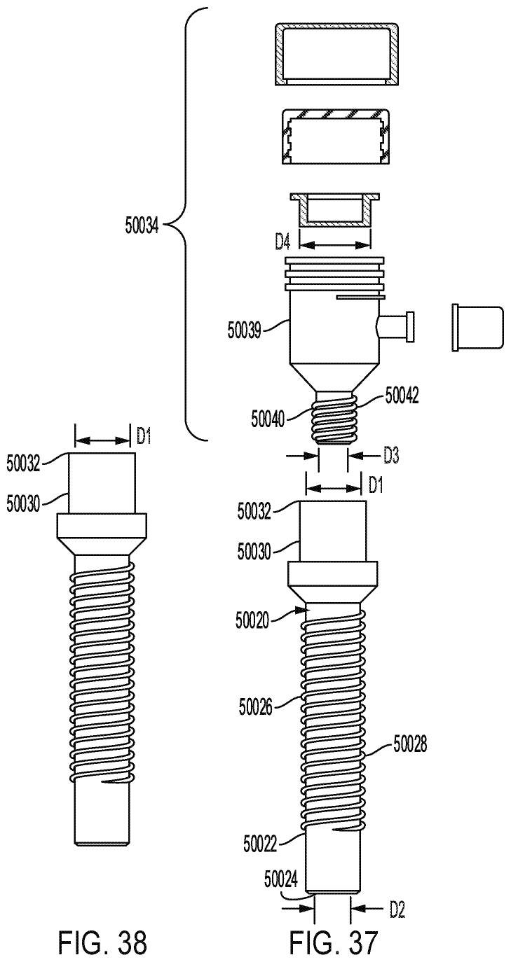

In FIG. 37, an invasive portion 50020 comprises a cylindrical section 50022 having a central passageway 50024. An invasive portion retainer 50026 is located on an outer surface 50028 of the invasive portion. The invasive portion retainer functions to retain the invasive portion within the patient during surgery. In the embodiment, the invasive portion retainer comprises threads helically surrounding the outer surface. Other invasive portion retainers will be obvious to those skilled in the art. The invasive portion 50020 further comprises an invasive portion coupler 50030 at its axially outer end 50032. In the embodiment of the device, the invasive portion coupler 50030 comprises a threaded receptacle having an internal diameter D1.

A non-invasive portion 50034 adaptively couples to the invasive portion 50020 at the coupler 50030 by matching threads. The non-invasive portion has a cylindrical main section 50039 of an internal diameter D4, larger than the diameter D2 of the central passageway 50024. The non-invasive portion 50034 tapers to a narrow section 50040, where threads 50042 are located. The narrow section has an internal diameter D3 larger than or equal to the internal diameter D2 of the central passageway and an external threaded diameter which firmly threadably engages into the threads of the internal diameter D1 of coupler 50030. Reference may be made to U.S. Patent Application Serial No. 25,024, now U.S. Pat. No. 5,383,860, the entire contents of which are incorporated herein by reference, for additional detailed discussion.

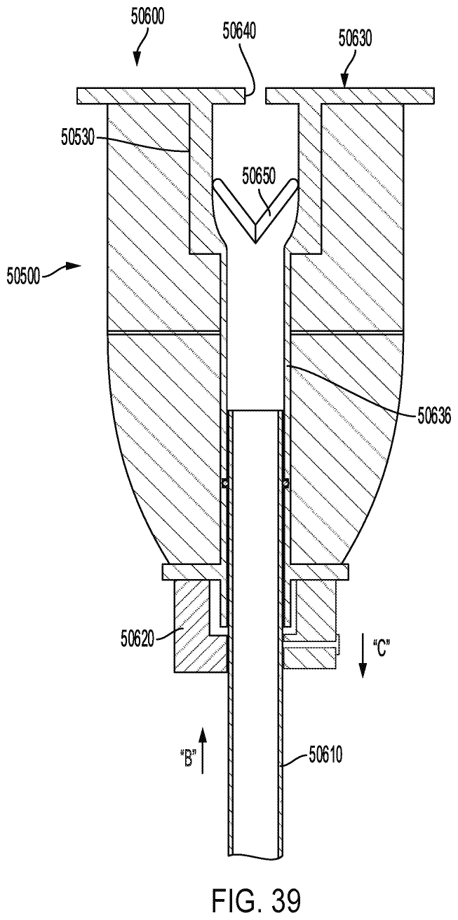

Referring to FIG. 39, cannula assembly 50600 is shown extending through mounting structure 50500. Cannula assembly 50600 includes a cannula or trocar 50610, an attachment member 50620, a barrier 50630, a first seal 50640, and a second seal 50650. Generally, cannula assembly 50600 is configured to provide a passageway for a surgical instrument (e.g., surgical instrument) to be inserted through an incision in a patient's skin and adjacent target tissue. Additionally, the cannula assembly 50600 is configured to minimize or prevent gasses and/or fluids from exiting the patient proximally through cannula assembly 50600, for example.

Cannula 50610 is an elongated, hollow tube that is configured to allow an elongated portion and an end effector of a surgical instrument to pass therethrough and access target tissue within a patient, for example. Cannula 50610 is sized and dimensioned for insertion within a channel 50530 of mounting structure 50500. More particularly, cannula 50610 is configured to be inserted into channel 50530 of mounting structure 50500 in a distal-to-proximal direction (in the general direction of arrow “B” in FIG. 39), and cannula 50610 may be removed from channel 50530 in a proximal-to-distal direction (in the general direction of arrow “C” in FIG. 39). An outer diameter of cannula 50610 and an inner diameter of barrier 50630 (e.g., a distal cylindrical section 50636) within channel 50530 of mounting structure 50500 may be similarly sized to enable a frictional engagement therebetween. Reference may be made to International Application Patent Application Serial No. PCT/US2017/034178, now International Publication No. WO/2017/205467, the entire contents of which are incorporated herein by reference, for additional detailed discussion.

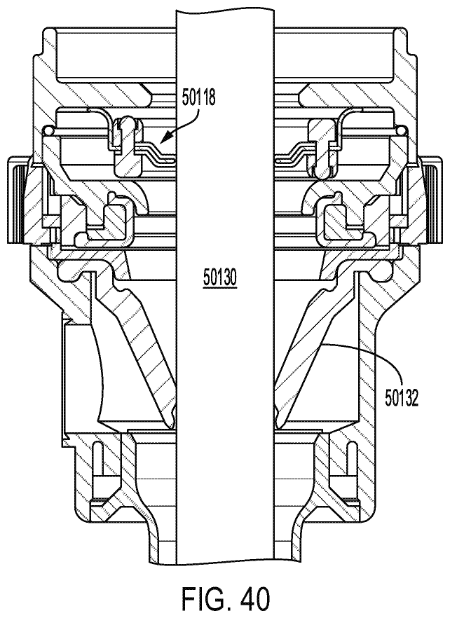

FIG. 40 shows a shaft 50130 of a surgical instrument, such as trocar obturator, inserted through seal assembly 50100 and a duck bill valve or “zero” seal valve 50132 which prevents the escape of insufflation gases in the absence of an instrument in the trocar assembly. As shown in FIG. 40, seal member 50118 provides a seal about the periphery of instrument shaft 50130. Reference may be made to U.S. patent application Ser. No. 11/786,251, now U.S. Patent Application Publication No. 2007/0197972, the entire contents of which are incorporated herein by reference, for additional detailed discussion.

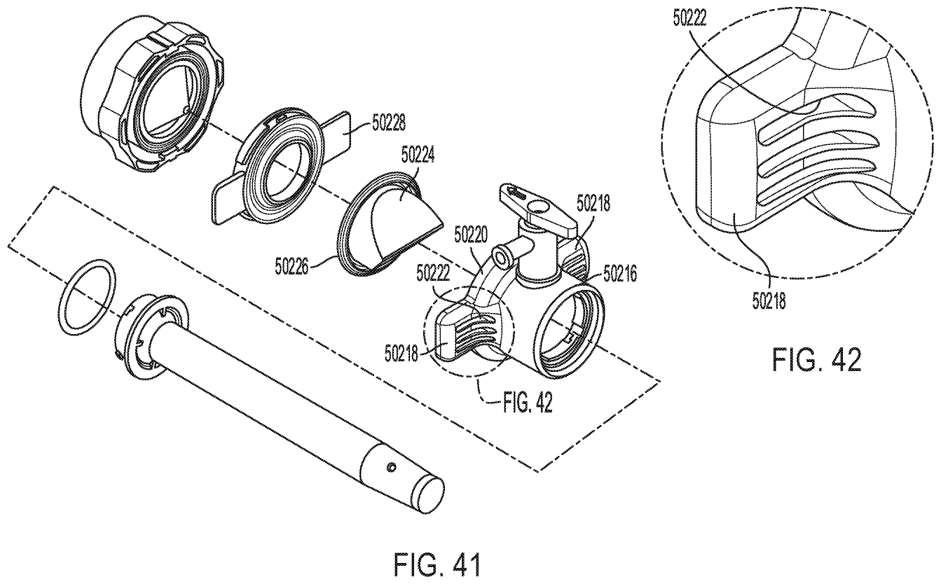

Referring now to FIGS. 41 and 42, a cannula assembly of modular trocar system will now be described. Cannula assembly includes a molded cylindrical base portion 50216 having transversely extending grip portions 50218 formed to extend from an annular flange formed at the proximal end of cylindrical base 50216. A series of slots 50222 are formed along the underside or distal side of grips 50218.

Slots 50222 are particularly advantageous in two respects. First, in assembling cannula assembly, there are three basic principle components: cylindrical base portion 50216 having outwardly directing finger grips 50218, a duck bill valve element 50224 having a flange 50226 which is configured and dimensioned to rest on annular flange 50220 of cylindrical base portion 50216 and a cannula housing cover portion such as proximal housing element 50228 which is configured and dimensioned to rest on duck bill flange 50226 and within the outwardly directed finger grips 50218. It has been found that by coring out the underside of outwardly extending finger grips 50218 with parallel slots 50222, molding sinks which had been previously forming on the proximal side of outwardly extending fingers 50218 of cylindrical base portion 50216 were significantly reduced, thereby providing a much more reliable flat surface, against which duck bill flange 50226 may rest and against which the upper or proximal housing element 50228 may be welded. Reference may be made to U.S. patent application Ser. No. 09/140,076, now U.S. Pat. No. 5,980,493, the entire contents of which are incorporated herein by reference, for additional detailed discussion.

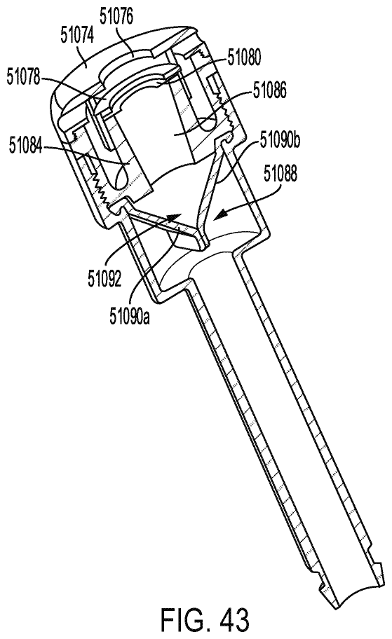

FIG. 43 shows the internal components of the sealing cannula. As shown in FIG. 43, the sealing cannula comprises a cannula cap 51074 having an access orifice 51076 formed thereon positioned on the upper cannula body. The cannula cap 51074 may be attachable to the upper cannula body in a variety of ways, including for example, in snap fit, screw relation, or adhesively joined. An o-ring 51078 and sealing washer 51080 defining a washer orifice are positioned proximal the cannula cap 51074, and act as a sealing conduit between the cannula cap 51074 and the guide member lumen 51086 formed in the guide member 51084. The guide member 51084 is attached to the upper cannula body in screw-like fashion. In alternative embodiments, the guide member 51084 may be attached to the upper cannula body 51066 in slip-fit relation, snap-fit relation, or other manners known in the art. As shown in FIG. 43, the guide member lumen 51086 is tapered. In another embodiment the walls of the guide member 51084 forming the guide member lumen 51086 maybe substantially parallel.

The embodiment further comprises a sealing member 51088 located within the lower cannula body and in communication with the guide member lumen 51086 and the device channel. The sealing member 51088 prevents a backflow of blood or other material from entering the cannula. As shown, the sealing member 51088 comprises a duckbill seal 51090 having at least two sealing leafs 51090a and 51090b forming a sealing receiver 51092. In alternative embodiments, various sealing devices may be incorporated into the sealing cannula, including, for example, sealing irises and flapper valve devices. Reference may be made to U.S. patent application Ser. No. 09/800,390, now U.S. Pat. No. 6,537,290, the entire contents of which are incorporated herein by reference, for additional detailed discussion.

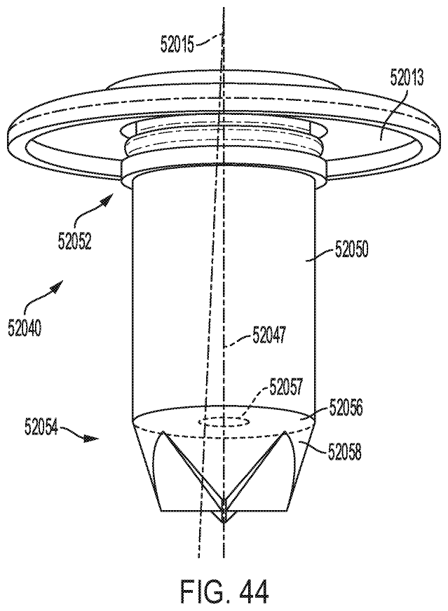

With reference to FIG. 44, an embodiment comprises a suspended, pendent valve module 52040 which can be mounted to an end cap 52013, within a trocar housing, and adapted to receive a wide range of instrument sizes. As illustrated in FIG. 44, the end cap 52013 is typically disposed in a radial plain generally perpendicular to the axis 52015 of the trocar. The module 52040 also has an axis 52047 and is characterized by an elongate tube 52050 having a proximal end 52052 and a distal end 52054. In an embodiment, the proximal end 52052 is coupled to the end cap 52013, while the distal end 52054 carries a septum valve 52056 with an orifice 52057, and a zero valve 52058.

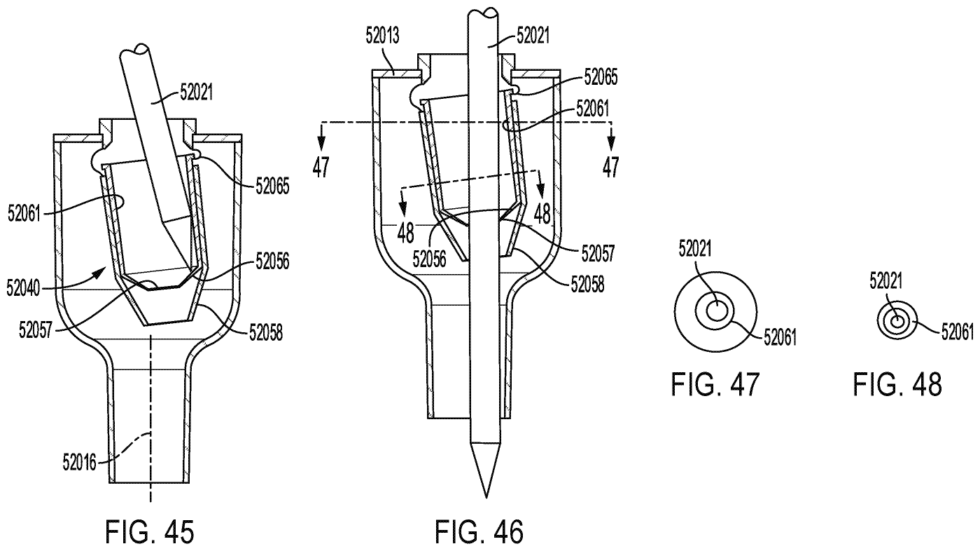

As illustrated in FIG. 45, an instrument 52021 will often be introduced at some angle to the axis 52016 which will cause it to contact the inner surface of the tubular member 52061. This will cause the pendent valve module 52040 to pivot at the flexible coupler 52065, thereby moving the septum valve 52056 and its orifice 52057 toward the distal tip of the instrument 52021. If this tip contacts the frusto-conical edges of the valve 52056, it would do so at a face angle which causes the orifice 52057 to move further toward the instrument 52021. This face angle is advantageously increased due to the pendulating characteristics of the module 52040.

In this case, the highly flexible coupler 52065 of the second tubular member comprises a series of thin, convoluted, folded or corrugated features that allow the pendulous seal module 52040 to move from side-to-side, to bend, to rotate or otherwise to be positioned by the inserted or approaching instrument 52021. An additional embodiment of the highly flexible coupler 52065 may comprise a thin material that stretches and folds to achieve the same goals. An additional embodiment of the highly flexible coupler 52065 may include a support region made of a low durometer material that achieves the same goals. Reference may be made to U.S. patent application Ser. No. 11/423,819, now U.S. Pat. No. 8,613,727, the entire contents of which are incorporated herein by reference, for additional detailed discussion.

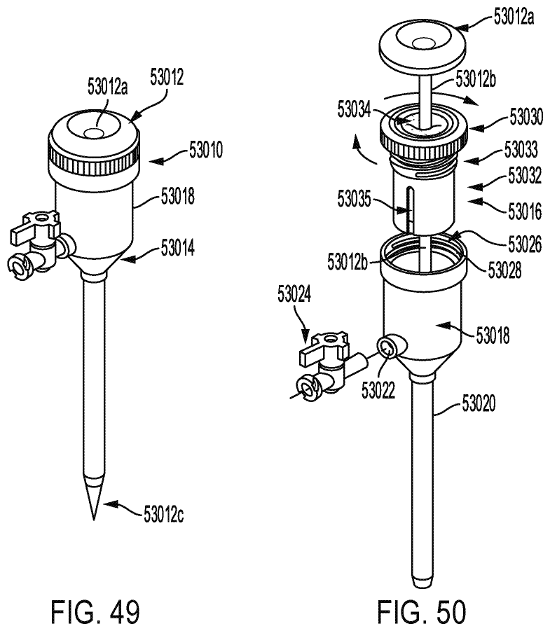

Referring now to FIGS. 49 and 50, an assembled trocar 53010 comprises a trocar obturator 53012, a trocar tube 53014, and a valve cartridge 53016. The trocar obturator comprises a head 53012a, an elongate shaft 53012b extending downwardly from the head and terminating in a trocar tip 53012c. The trocar tube includes an upper shell or housing 53018 and a depending tube 53020 through which pass the trocar obturator and surgical instruments (not shown) for endoscopic surgery. The housing has a port 53022 (which may be fitted with a stop clock 53024) used for insufflating and desufflating an abdominal cavity, for example, through the trocar tube. The upper shell is shown cylindrical in shape, however, it can be any suitable shape, box-like for example. The shell has an opening 53026 at its upper end and has an internally threaded flange 53028 or other suitable fastening means to receive and secure the valve cartridge 53016.

The cartridge comprises an upper collar 53030 and a depending cylindrical skirt 53032 for receiving and positioning primary 53034 and secondary seals and the protective insert 53038 for the secondary seal, for attachment to the shell, for defining a sealed axial passage for the trocar as well as instruments passed through the trocar tube, and for admitting peritoneal pressure to the exterior surfaces of the secondary seal. The exterior and interior elements of the cartridge assembly are shown in FIG. 50 and includes cap or collar 53030 and subjacent threaded section 53033 for securing the cartridge to the trocar shell. The remaining skirt portion 53032 of the cartridge is long enough to cover entirely the secondary seal valve while having vents 53035 for the purpose of admitting peritoneal pressure to the exterior surface of the secondary seal. The vents are in the form of slits extending upward from the bottom edge of the skirt best shown in FIG. 50, it being understood that other shaped openings in the skirt may be used for venting. Reference may be made to U.S. Patent Application Serial No. 523,108, now U.S. Pat. No. 5,662,615, the entire contents of which are incorporated herein by reference, for additional detailed discussion.

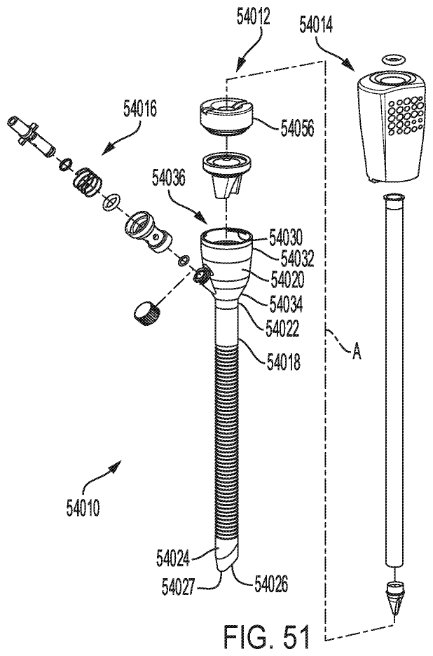

Referring now to FIG. 51, one embodiment of a disclosed trocar assembly, generally designated 54010, may include a sleeve assembly 54012 and an obturator assembly 54014. Optionally, as will be discussed in greater detail below, the trocar assembly 54010 may additionally include an insufflation valve assembly 54016 coupled to the sleeve assembly 54012.

The sleeve assembly 54012 may include a generally cylindrical or tubular cannula 54018, a generally annular housing 54020, a channel seal 54050 received in the housing 54020 and a cap 54056 attached to the housing 54020. The cannula 54018 may be elongated along a longitudinal axis A, and may include an open proximal end 54022 and an open distal end 54024. The open distal end 54024 may included a bevel 54026 that terminates in a pointed tip 54027. A lumen may extend along the axial length of the cannula 54018 between the open proximal end 54022 and the open distal end 54024. The housing 54020 may define an internal volume 54030 and may include an open proximal end 54032 and an open distal end 54034. The open distal end 54034 of the housing 54020 may be connected to the open proximal end 54022 of the cannula 54018 to couple the internal volume 54030 of the housing 54020 with the lumen of the cannula 54018, thereby defining an elongated working channel 54036 that extends axially through the sleeve assembly 54012. Reference may be made to U.S. patent application Ser. No. 12/575,598, now U.S. Pat. No. 8,491,533, the entire contents of which are incorporated herein by reference, for additional detailed discussion.

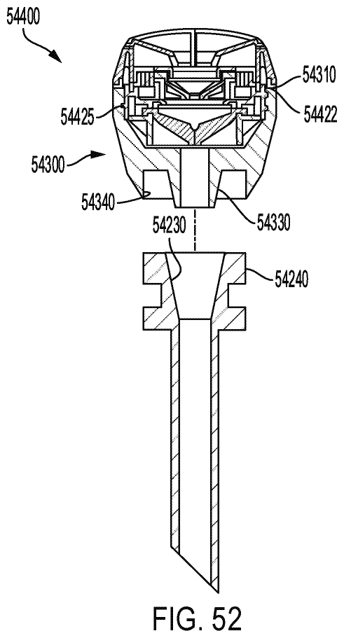

FIG. 52 shows an adaptor 54300 connected to seal assembly 54400. Threading of adaptor (54300) is connected to threading 54425 of seal assembly 54400. Through this engagement of threading, connections between distal surface 54422 of seal assembly 54400 and proximal end 54310 of adaptor 54300 are sufficient to provide a seal maintaining pneumostasis in an insufflated body cavity of a patient. Also, seal assembly housing is supporting seal assembly 54400, so that if adaptor 54300 is stable, so is seal assembly 54400.

A seal between adaptor 54300 and flange 54240 maintaining pneumostasis can be created using multiple materials, if adaptor 54300 is made entirely out of elastomeric material, interior taper 54330 could be dimensioned for interference with interior wail defining proximal angled opening 54230, exterior taper 54340 could be dimensioned for interference with flange 54240, or both interior taper 54330 and exterior taper 54340 could be dimensioned for interference with interior wail defining proximal angled opening 54230 and flange 54240 respectively. All of these possibilities could create a seal, in effect maintaining pneumostasis in an insufflated body cavity of a patient. Reference may be made to International Application Patent Application Serial No. PCT/US2015/065493, now International Publication No. WO/2016/100181, the entire contents of which are incorporated herein by reference, for additional detailed discussion.

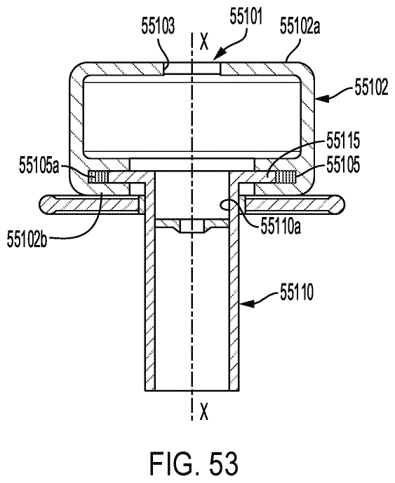

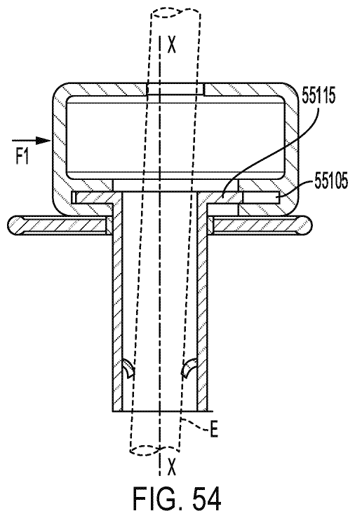

Turning now to FIG. 53, housing 55102 forms a substantially annular member having a partially closed proximal end 55102a and a partially closed distal end 55102b. Housing 55102 may instead define an oval, square, rectangular or other suitable profile. Housing 55102 may be constructed of metal, plastic, polymer or other suitable material. Housing 55102 defines a passageway 55101 therethrough for receiving an instrument E (FIG. 54). Proximal end 55102a of housing 55102 defines an opening 55103 configured to receive instrument E therethrough.

Still referring to FIG. 53, distal end 55102b of housing 55102 defines a horizontal slot 55105 for receiving a flange 55115 formed on proximal end 55110a of cannula 55110. As will be discussed in further detail below, distal end 55102b of housing 55102 is configured to floatingly receive flange 55115 therein. A rubber or foam ring 55105a may be included with slot 55105. In the event that housing 55102 is pressurized, foam ring 55105a may form a seal between the connection of housing 55102 and cannula 55110 to prevent leakage of the insufflation gas.

Referring to FIG. 54, when a user applies a lateral force against housing 55102 in a direction indicated by arrow F1, housing 55102 translates horizontally relative to cannula 55110 in the direction force F1. Slot 55105 formed in distal end 55102b of housing 55102 permits this movement of housing 55102 relative to cannula 55110. In this manner, housing 55102 and thus, instrument E inserted therethrough, may be manipulated relative to cannula 55110 without moving cannula 55110. As discussed above, slot 55105 may include a rubber or foam ring 55105a that may compress as housing 55102 is moved about cannula 55110. The release of force F1 against housing 55102 causes ring 55105a to decompress, thereby returning housing 55102 it its initial, concentric position with cannula 55110. In this manner, housing 55102 may be moved any direction in a horizontal plane relative to cannula 55110. By being able to laterally move housing 55102 relative to cannula 55110, a user may more precisely manipulate and have greater control over instrument E inserted therethrough.

During a surgical procedure, a trocar assembly can be introduced into a patient's abdominal wall to provide access to the patient's abdominal cavity. Surgical instruments can be inserted through the trocar assembly and into the abdominal cavity to perform laparoscopic surgical procedures. During a laparoscopic surgical procedure, the abdominal cavity is generally insufflated in order to increase the volume of the working environment. A seal system can be provided in the trocar assembly to inhibit the escape of the insufflation gases therethough. After the surgical procedure, the trocar assembly is typically discarded. It is desirable that a portion of the trocar assembly be reprocessable and reusable for more than one surgical procedure to reduce the costs of performing surgical procedures.

Referring now to FIG. 55, a trocar assembly 13700 is provided that includes a reusable housing 13705. The reusable housing 13705 is made of a material that is able to be sterilized and used for more than one surgical procedure, such as metal. The reusable housing 13705 includes a robot arm holding feature 13710 defined around the outside perimeter of the reusable housing 13705. The robot arm holding feature 13710 is sized and configured to be grasped by an arm of a robotic surgical system. The robot arm holding feature 13710 allows for proper alignment between the arm of the robotic surgical system and the robot arm holding feature of the reusable housing 13705.

The reusable housing 13705 of the trocar assembly includes a distal end 13715 that includes a threaded portion 13720. The threaded portion 13720 is configured to releasably couple the reusable housing 13705 to a disposable cannula 13725 that includes a threaded portion 13730, as is shown in FIG. 55. The disposable cannula 13725 can made of a material that is intended to be disposed of at the conclusion of a surgical procedure, such as plastic. The cannula 13725 is configured to be coupled to the reusable housing 13705 before, or during, a surgical procedure and removed and disposed of after the completion of the surgical procedure. The cannula 13725 facilitates passage of a surgical instrument through the reusable housing 13705 and into a patient.

The reusable housing 13705 further includes a proximal end 13735 defining a proximal opening 13740 into the reusable housing 13705. The proximal opening 13740 is configured to receive an insertable seal system 13750 that includes a first seal 13750a and a second seal 13750b.

The second seal 13750b of the insertable seal system 13750 is configured to be inserted into the proximal opening 13740 of the reusable housing 13705 before the first seal 13750a. In one example, the second seal 13750b is configured as a “no-instrument seal”, such as a duckbill seal, as illustrated in FIG. 55. A no-instrument seal is a seal that is configured to prevent insufflation gases from escaping a patient's abdominal cavity in the absence of a surgical instrument. As is shown in FIG. 55, the distal end 13752 of the second seal 13750b is biased to a closed position when a surgical instrument is not inserted therethrough. While a duckbill seal is illustrated and described, it is also contemplated that a pendulum seal, such as the pendant valve module described in U.S. patent application Ser. No. 11/423,819, now U.S. Pat. No. 8,613,727, the entire contents of which are incorporated herein by reference, can be utilized.

The duckbill seal 13750b includes a flange 13755 that is configured to seat upon a shoulder 13760 of the reusable housing 13705. The flange 13755 illustrated in FIG. 55 is configured such that, when seated upon the shoulder 13760 of the reusable housing 13705, a clearance gap is formed between the flange and an inside wall 13757 of the reusable housing 13705. In a separate embodiment, the flange 13755 can be configured to extend and make contact with the inside wall 13757 of the reusable housing 13705, leaving no clearance gap therebetween.

The first seal 13750a of the insertable seal system 13750 is configured to be inserted into the proximal opening 13740 of the reusable housing 13705 and positioned on top of the second seal 13750b. The first seal 13750a includes an instrument lip seal 13762, which is configured to prevent insufflation gases from escaping the patient's abdominal cavity in the presence of a surgical instrument. Specifically, when a surgical instrument is inserted through the instrument lip seal 13762, the instrument lip seal 13762 is configured to maintain contact with the surgical instrument and prevent insufflation gases from escaping through the first seal 13750a. The first seal 13750a and the second seal 13750b cooperatively function to provide that, in either the absence or the presence of a surgical instrument, insufflation gases can be sealed in the patient's abdominal cavity.

The first seal 13750a includes an outer sleeve 13765 made of polyisoprene. The first seal 13750a further includes a thin-walled polycarbonate cylinder 13770 configured to be positioned inside of the outer sleeve 13765 to provide structural support to the outer sleeve 13765. The thin-walled cylinder 13770 can have a thickness of approximately 0.02″. In one example, the thin-walled cylinder can 13770 can have a thickness in the range of 0.01″ and 0.03″. In another example, the thin-walled cylinder can 13770 can have a thickness in the range of 0.015″ and 0.025″.

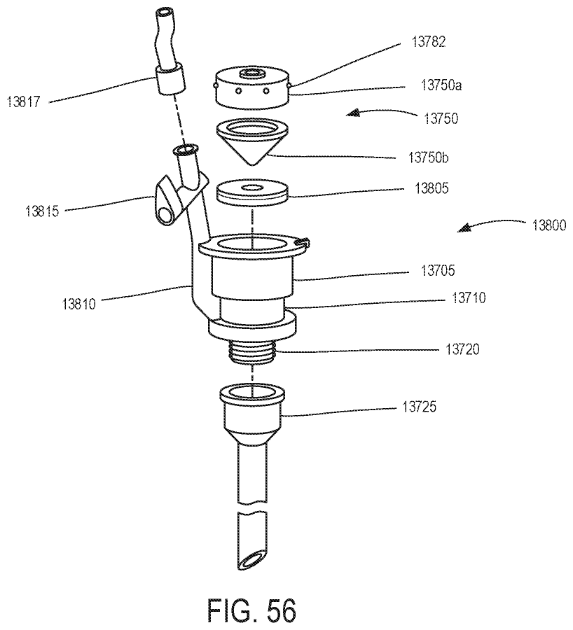

As described above, the first seal 13750a of insertable seal system 13750 is configured to be inserted into the proximal opening 13740 of reusable housing 13705 and positioned on top of the second seal 13750b. An exterior surface of the first seal 13750a can include an interference lip 13780 extending laterally around the perimeter of the first seal 13750a. The interference lip 13780 is configured to extend from the first seal 13750a and contact the inner wall 13757 of the reusable housing 13705 to create an additional seal to prevent insufflation gases from escaping the patient. The interference lip 13780 can be configured as a continuous member such that the interference lip 13780 makes contact with the inner wall 13757 of the reusable housing 13705 around the entire perimeter of the first seal 13750a. In a separate embodiment, referring briefly to FIG. 56, the interference lip can include a plurality of interference lip members 13782 positioned at discrete points around the perimeter of the first seal 13750a. The interference lip 13780 and interference lip members 13782 can be configured to maintain the position of the first seal 13750a within the reusable housing 13705 and relative to the second seal 13750b.

The bottom surface of the first seal 13750a can also include a ridge member 13790 configured to extend around the bottom surface of the first seal 13750a and contact a top surface of the second seal 13750b. The ridge member 13790 is configured to maintain the first seal 13750a in seating alignment with the second seal 13750b, as well as provide an additional seal to prevent insufflation gases from escaping the patient. Similar to the interference lip 13780 described above, in one embodiment, the ridge member 13790 can be configured to extend continuously around the bottom the bottom surface of the first seal 13750a. In another embodiment, the ridge member can include a plurality of ridge members configured to contact the top surface of the second seal at a discrete number of points.

During a surgical procedure, surgical instruments are susceptible to being covered in bodily fluids and other biological materials, such as blood. In use with the trocar assembly described above, as the surgical instrument is removed from the patient's abdomen, the surgical instrument passes through the second seal (duckbill seal) and then the first seal (instrument lip seal) before being removed from the trocar assembly. While passing through the first seal and the second seal, bodily fluids and other biological materials can scrape against points of contact with the seals and be left behind on the seals. As a result, when the surgical instrument is reintroduced into the trocar assembly, the surgical instrument may contact and be covered in these left behind bodily fluids and other biological materials. This can interfere with laparoscopic imaging devices, such as a camera, where it is important that the lens of the imaging device remain clean so that a clinician can properly visualize the surgical procedure. Biological material will accumulate onto the camera during introduction into the patient's abdominal cavity, thus, obstructing the clinician's view. A need exists to ensure that the seals of a trocar assembly remain clean of bodily fluids and other biological materials when a surgical instrument is removed though the trocar assembly.

Referring now to FIG. 56, an exploded view of a trocar assembly 13800 is shown. The trocar assembly 13800 shown in FIG. 56 is similar in many respects to the trocar assembly shown and described in FIG. 55. The trocar assembly 13800 shown in FIG. 56, however, includes a third 13805 seal for use with an insertable seal system 13750. The third seal 13805 is configured to be positioned in a reusable housing 13705 of the trocar assembly 13800 prior to insertion of the insertable seal system 13750. The third seal 13805 is configured as a scraper seal, which is configured to wipe, wick, and absorb fluids from a surgical instrument as the surgical instrument is being removed from a patient and before the surgical instrument reaches the insertable seal system 13750, functioning to keep the first seal 13750a and the second seal 13750b clean. The third seal is configured to distribute the accumulated biological material away from the center of the third seal 13805 such that the surgical instrument would not contact the accumulated biological material as the surgical instrument passes through the third seal 13805 and into the patient. As an example, distribution of the biological material away from the center of the third seal 13805 allows that an imagining device will remain clean as it is inserted through the third seal 13805 and into a patient, thus allowing for an unobstructed view during a procedure.

Referring still to FIG. 56, the trocar assembly 13800 can include an insufflation port 13810 configured to extend from the reusable housing 13705. The insufflation port 13810 can facilitate passage of insufflation gases into a patient's abdominal cavity to increase the working environment during a surgical procedure. The insufflation port 13810 can further include a lever 13815, which can transition the insufflation port 13810 between an open configuration and a closed configuration. While a lever is illustrated, other means of transitioning the insufflation port between the open configuration and the closed configuration are contemplated, such as with a button or a valve, as an example. In the open configuration, a clinician is able to pass insufflation gases through the insufflation port 13810 and into a patient abdominal cavity. In the closed configuration, the insufflation port 13810 is sealed such that insufflation gases may not escape through the insufflation port 13810. The insufflation port 13810 can further be configured to couple to a luer lock 13817, which can facilitate insufflation gases from an insufflation source into the insufflation port 13810.

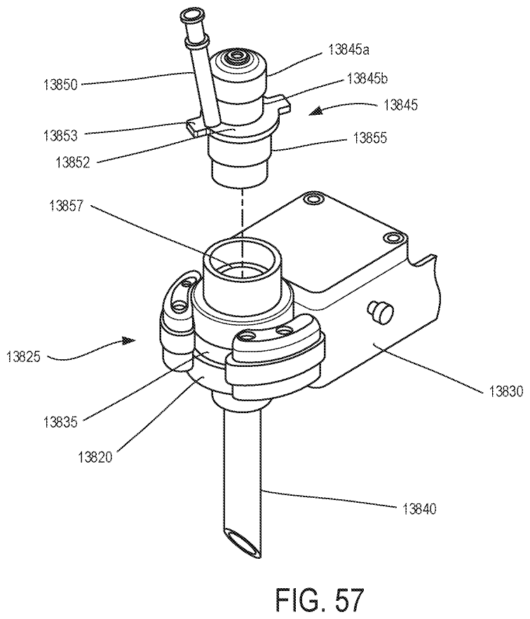

Referring now to FIG. 57, a reusable housing 13820 of a trocar assembly 13825 is shown being fixably held by an arm 13830 of a robotic surgical system. The reusable housing 13820 is aligned with the arm 13830 of the robotic surgical system by way of the robot arm holding feature 13835 described above. A cannula 13840 is attached to a distal end of the reusable housing 13820. In one example, the cannula can be attached to the reusable housing 13820 by way of mating threads between the cannula and the reusable housing, described above. While coupling the cannula 13840 and the reusable housing 13820 by way of threads has been described, other ways of coupling the cannula 13840 and the reusable housing 13820 are envisioned, such as by snap-fit, press-fit, or other ways of coupling two members.

A seal assembly 13845 is shown that is positionable in the reusable housing 13820 of the trocar assembly 13825. The seal assembly 13845 can include a first seal 13845a and a second seal 13845b, such as the instrument lip seal and duckbill seal, respectively, as described above. The seal assembly 13845 can also include an insufflation port 13850, which will be described in more detail below. The seal assembly 13845 can further include a gripping feature 13852 configured to assist in positioning the seal assembly 13845 into the reusable housing 13820. In one example, the gripping feature can include two contact members 13853 extending away from the seal assembly 13845 in opposite directions. While two contact members 13853 are shown, more of less than two contact members 13853 can be used.

The seal assembly 13845 further includes a rigid coupling feature 13855 extending from a bottom surface of the seal assembly 13845. In one example, the coupling feature can be made of plastic. The coupling feature 13855 includes a stepped configuration that is configured to mate with a stepped configuration 13857 on an inside surface of the reusable housing 13820. The stepped configuration between the coupling feature 13855 and the stepping configuration 13857 on the inside surface of the reusable housing 13820 provides for a proper alignment between the seal assembly 13845 and the reusable housing 13820. When the seal assembly 13845 is seated within the reusable housing 13820, the reusable housing 13820 floatingly supports the seal assembly 13845. The floating support allows the seal assembly 13845 to adjust relative to the reusable housing 13820 and the cannula 13840 as surgical instruments are inserted and removed from the patient's abdomen. In another embodiment, when the seal assembly 13845 is seated within the reusable housing 13820, the reusable housing 13820 rigidly supports the seal assembly 13845 such that the seal assembly 13845 cannot adjust relative to the reusable housing 13820 and the cannula 13840 as surgical instruments are inserted and removed from the patient's abdomen.

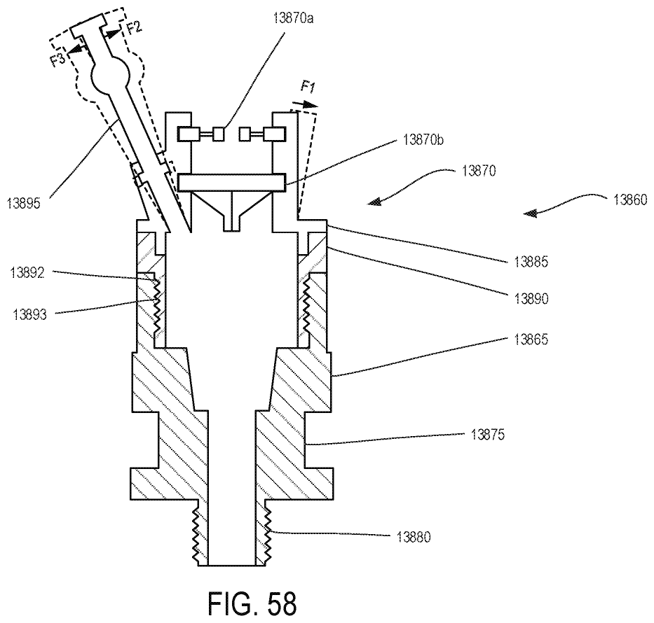

Referring now to FIG. 58, another embodiment of a trocar assembly 13860 is shown. The trocar assembly 13860 is shown including a reusable housing 13865 and a seal assembly 13870. The reusable housing 13865 includes a robot arm holding feature 13875 configured to be grasped by an arm of a robotic surgical system and threads 13880 to threadably engage a disposable cannula (not shown). The seal assembly 13870 includes an elastomer seal housing 13885 configured to house internal components of the seal assembly 13870. The elastomer seal housing 13885 is configured to be flexible, such that, when the elastomer seal housing 13885 experiences outside forces (F1 as an example), the elastomer seal housing 13885 can transition from an unflexed configuration into flexed configuration (illustrated by dotted lines on FIG. 58). Once an outside force is removed, the elastomer seal housing 13885 can return to the unflexed configuration.

The seal assembly 13870 further includes a first seal 13870a and a second seal 13870b. The first seal 13870a, such as an instrument lip seal, is configured to prevent insufflation gases from escaping the patient's abdomen when a surgical instrument is present through the first seal. The second seal 13870b, such as a duckbill seal, is configured to prevent insufflation gases from escaping the patient abdomen when a surgical instrument is not present through the second seal.

The seal assembly 13870 also includes a rigid seal housing 13890 extending from a bottom surface of the elastomer seal housing 13885. The rigid seal housing 13890 includes a threaded portion 13982 that is configured to engage a threaded portion 13983 on an inside surface of the reusable housing 13865 to bring the seal assembly 13870 into threaded engagement with the reusable housing 13865. While coupling the reusable housing 13865 and the seal assembly 13870 by way of threads has been described, other embodiments are envisioned where the reusable housing 13865 and the seal assembly 13870 are coupled by way of snap-fit or press-fit connections, or by another suitable connections.

The seal assembly 13870 further includes an insufflation port 13895 extending from the elastomer seal housing 13885. The insufflation port 13895 is configured to bypass the first seal 13870a and the second seal 13870b of the seal assembly 13870 to provide access into the patient's abdomen. The insufflation port is configurable such that an insufflation stop cock is couplable therewith for use during a surgical procedure. The insufflation stop cock prevents insufflation gases from escaping the patient's abdominal cavity via the insufflation port during the surgical procedure. In a first embodiment, the insufflation port 13895 can be rigid. In a second embodiment, the insufflation port 13985 can be flexible, similar to the elastomer seal housing 13885 such that when the insufflation port 13895 experiences outside forces (F2 and F3 as an example), the elastomer seal housing 13885 can transition from an unflexed configuration into flexed configuration (illustrated by dotted lines on FIG. 58)

As described above, the elastomer seal housing 13885 is movable from an unflexed configured to a flexed configuration when the elastomer seal housing 13885 experiences an outside force. In one example, the robot arm of the robotic surgical system pivots the reusable housing 13865 towards the side of the seal assembly 13870 that includes the insufflation port 13895 and the insufflation port 13895 make contact with an exterior body, such as the patient's abdomen. In an example where the insufflation port 13895 is rigid, the insufflation port 13895 would contact the exterior body and pivot away from the exterior body as a result of the elastomer seal housing 13885 being flexible, reducing trauma on the patient. In an example where the insufflation port 13895 is flexible, the insufflation port 13895 would contact the exterior body and one or both of the insufflation port 13895 and the elastomer seal housing 13885 would flex away from the exterior body. Because of the configuration between the insufflation port 13895 and the elastomer seal housing 13885, the seal on an instrument and the guidance of the instrument into the patient's abdomen would be maintained.

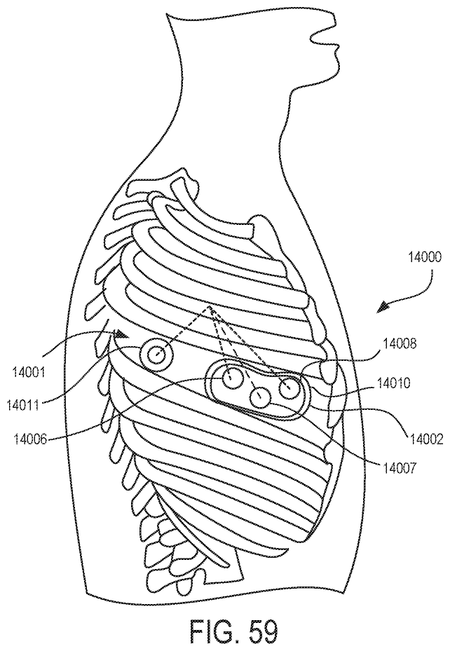

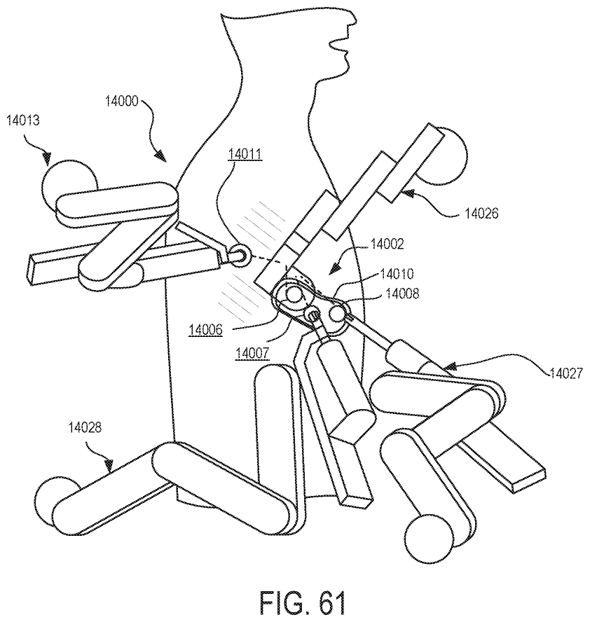

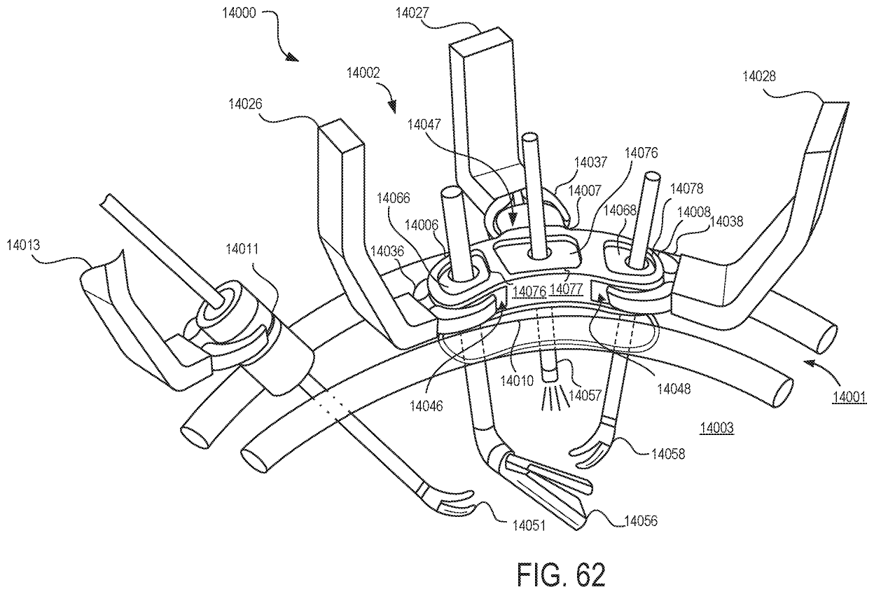

Referring primarily to FIGS. 59-62, a minimally invasive surgical access system 14000 is utilized to perform a thoracic surgery. FIG. 59 illustrates an example surgical access device 14002 of the surgical access system 14000 positioned at the fifth intercostal space 14001 of a patient. The surgical access device 14002 includes three access ports 14006, 14007, 14008 that provide minimally invasive passageways into a thoracic cavity 14003 (FIG. 62) of the patient for a variety of surgical tools. The access ports 14006, 14007, 14008 reside and move within an outer perimeter defined by an atraumatic outer housing 14010 of the surgical access device 14002. The access ports 14006, 14007, 14008 include docking portions 14046, 14047, 14048 for releasably coupling to robotic arms 14026, 14027, 14028, respectively, as illustrated in FIG. 62.

In various examples, a surgical access system may include a surgical access device with more or less than three access ports and more or less than three robotic arms. In one example, a surgical access system may include a surgical access device with four access ports and four robotic arms. In another example, a surgical access system may include a surgical access device with two access ports and two robotic arms. In another example, a surgical access system may include a surgical access device with two access ports and three robotic arms. In another example, a surgical access system may include a surgical access device with three access ports and two robotic arms.

Referring primarily to FIG. 62, robotic arms 14026, 14027, 14028 include surgical mounting devices 14036, 14037, 14038, which include clamping assemblies for releasably coupling to docking portions 14046, 14047, 14048 of the surgical access device 14002. The clamping assemblies of the surgical mounting devices 14036, 14037, 14038 are transitionable between an open configuration and a closed configuration to releasably couple to the docking portions 14046, 14047, 14048, respectively. Additional information about the construction and operation of surgical mounting devices are described in U.S. 2018/0177557, titled MOUNTING DEVICE FOR SURGICAL SYSTEMS AND METHOD OF USE, and filed Jun. 6, 2016, which is hereby incorporated by reference herein in its entirety.

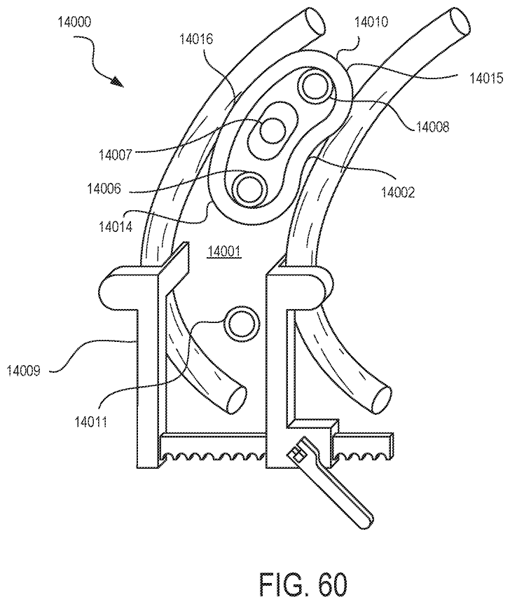

Referring to FIG. 60, to position the surgical access device 14002 at an intercostal space, an incision is made intercostally, or between two ribs of the left chest wall. A surgical retractor 14009 is then used to spread the ribs apart to accommodate the surgical access device 14002. A separate access port 14011 can also be placed intercostally a predetermined distance away from the surgical access device 14002, and can be releasably coupled to a fourth robotic arm 14013. In certain examples, the surgical retractor 14009 is integrated with the surgical access device 14002. In other examples, surgical retractor 14009 is separate from the surgical access device 14002.

In various aspects, as illustrated in FIG. 62, the atraumatic outer housing 14010 comprises a non-radial shape that corresponds to the shape of the ribs. In at least on example, the outer housing 14010 comprises a crescent shape. In at least on example, the outer housing 14010 comprises a general curvature that corresponds to the curvature of the ribs. The access ports 14006, 14007, 14008 are arranged along the curvature of the outer housing 14010. In the example of FIG. 60, the access ports 14006 and 14008 are located near ends 14014, 14015, respectively, of the outer housing 14010, while the access port 14007 is located near its apex 14016. Further, the access port 14007 is larger than the access ports 14006, 14008. It is, however, understood that the size, number, and/or arrangement of the access ports of a surgical access device 14002 can be selected to accommodate various surgical tools. In the example, illustrated in FIG. 62, a surgical stapler 14056 is received through the access port 14006, an imaging device 14057 is received through the access port 14007, and a surgical grasper 14058 is received through the access port 14008. An additional surgical grasper 14051 is received through the access port 14011 for triangulation with the surgical grasper 14058 and/or the surgical stapler 14056, for example.

Referring still to FIG. 62, the outer housing 14010 includes three compartments 14076, 14077, 14078 accommodating the access ports 14006, 14007, 14008, respectively. In various aspects, the access ports 14006, 14007, 14008 are movable within the compartments 14076, 14077, 14078 relative to the outer housing 14010. Further, the robotic arms 14026, 14027, 14028 are configured to cooperate to synchronously move the instruments 14056, 14057, 14058 relative to one another and/or relative to the surgical access device 14002.

In various aspects, the access ports 14006, 14007, 14008 include seal assemblies 14066, 14067, 14068, respectively, that may have one or more seals such as, for example, an iris seal and/or a duckbill seal configured to receive the instruments 14056, 14057, 14058, respectively. In various aspects, the docking portions 14046, 14047, 14048 are located at the seal assemblies 14066, 14067, 14068, and the robotic arms 14026, 14027, 14028 are configured to releasably couple to the docking portions 14046, 14047, 14048 to define remote centers for the instruments 14056, 14057, 14058 at the seal assemblies 14066, 14067, 14068, respectively. Further, the robotic arms 14026, 14027, 14028 are configured to cooperate to synchronously adjust the remote centers of the instruments 14056, 14057, 14058.

The seal assemblies 14066, 14067, 14068 permit the instruments 14056, 14057, 14058 to move within boundaries defined by the compartments 14076, 14077, 14078. Additional movement, however, requires a cooperative effort between the robotic arms 4026, 14027, 14028. Like the robotic arms 13002, 13003 (FIG. 4) the robotic arms 14026, 14027, 14028 may be driven by electric drives that are connected to the control device 13004 (FIG. 4). In various aspects, the control device 13004 automatically coordinates movement of the robotic arms 14026, 14027, 14028 in response to a user input concerning a subset of the robotic arms 14026, 14027, 14028. In other words, a user input for moving one of a plurality of robotic arms coupled to a multi-port surgical access device such as, for example, the surgical access device 14002 causes a control device such as, for example, the control device 13004 to synchronously move the plurality of robotic arms to comply with the user input.

In at least one example, to accommodate a user input to adjust a position of the surgical stapler 14056, the control device 13004 may cause the robotic arms 14026, 14027, 14028 to synchronously move to achieve the desired position of the surgical stapler 14056. The control device 13004 may further cause the imaging device 14057 and/or the surgical grasper 14058 to move relative to their respective seal assemblies 14067, 14068 to maintain their original orientations with respect to one another and/or with respect to a new orientation of the surgical stapler 14056. In various aspects, the control device 13004 may cause the robotic arms 14026, 14027, 14028 to synchronously move to adjust the surgical access device 14002 to a new orientation.

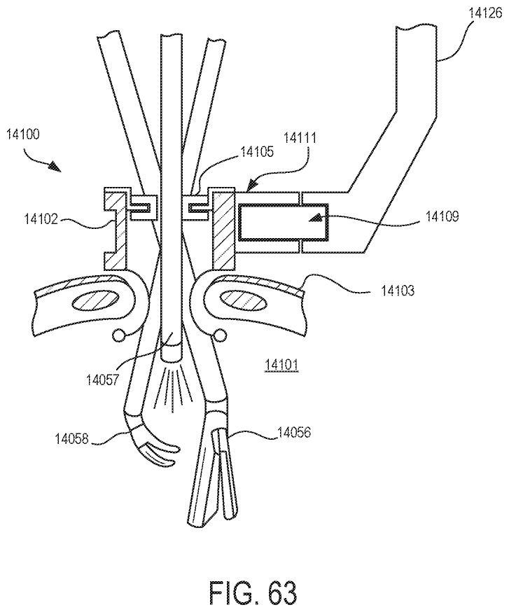

Referring now to FIG. 63, a surgical access device 14100 is similar in many respects to the surgical access device 14002. For example, the surgical access device 14100 is also configured to facilitate access to a body cavity 14101 through a body wall 14103 for the instruments 14056, 14057, 14058. However, the surgical access device 14100 includes only a single access port 14102 configured to accommodate a plurality of instruments such as, for example, the instruments 14056, 14057, 14058. In various aspects, the instruments 14056, 14057, 14058 are passed through a seal assembly 14105 defined in the access port 14102. The seal assembly 14105 includes one or more seals such as, for example, an iris seal and/or a duckbill seal.

The surgical access device 14100 is releasably coupled to a robotic arm 14126, which similar in many respects to the robotic arms 13002, 13003. For example, the robotic arm 14126 may be driven by electric drives that are connected to the control device 13004 (FIG. 4). Also, the robotic arm 14126 includes a mounting device 14109, which can be in the form of a clamp assembly, configured to releasably couple to a docking portion 14111 of the access port 14102.

In various instances, one of the instruments 14056, 14057, 14058 is controlled by the robotic arm 14126, while the other instruments are controlled by separate robotic arms. This arrangement permits the instruments 14056, 14057, 14058 to move relative to one another within a boundary defined by the seal assembly 14105, which permits instrument triangulation. As described in connection with the robotic arms 14026, 14027, 14028, a control device 13004 (FIG. 4) may respond to a user input concerning one of the robotic arms controlling the instruments 14056, 14057, 14058 by synchronously moving two or more of such robotic arms to comply with the user input.

In various instances, the instruments 14056, 14057, 14058 and the surgical access device 14100 are controlled by separate robotic arms. This arrangement permits the robotic arm 14126 to adjust a position and/or orientation of the surgical access device 14100 separately from the robotic arms controlling the instruments 14056, 14057, 14058. As described in connection with the robotic arms 14026, 14027, 14028, a control device 13004 (FIG. 4) may respond to a user input concerning one of the robotic arms controlling the instruments 14056, 14057, 14058 or the robotic arm 140100 by synchronously moving two or more of such robotic arms to comply with the user input.

The reader will appreciate that although FIG. 63 depicts three instruments inserted through the seal assembly 14105, this is not limiting. In certain examples, the seal assembly 14105 may accommodate two, three, four, or more instruments that may be controlled by separate robotic arms. Alternatively, multiple instruments can be controlled by the same robotic arm. For example, a robotic arm, releasably coupled to a surgical access device 14100, can be configured to support and move a plurality of instruments received through the seal assembly 14105 of the surgical access device 14100.

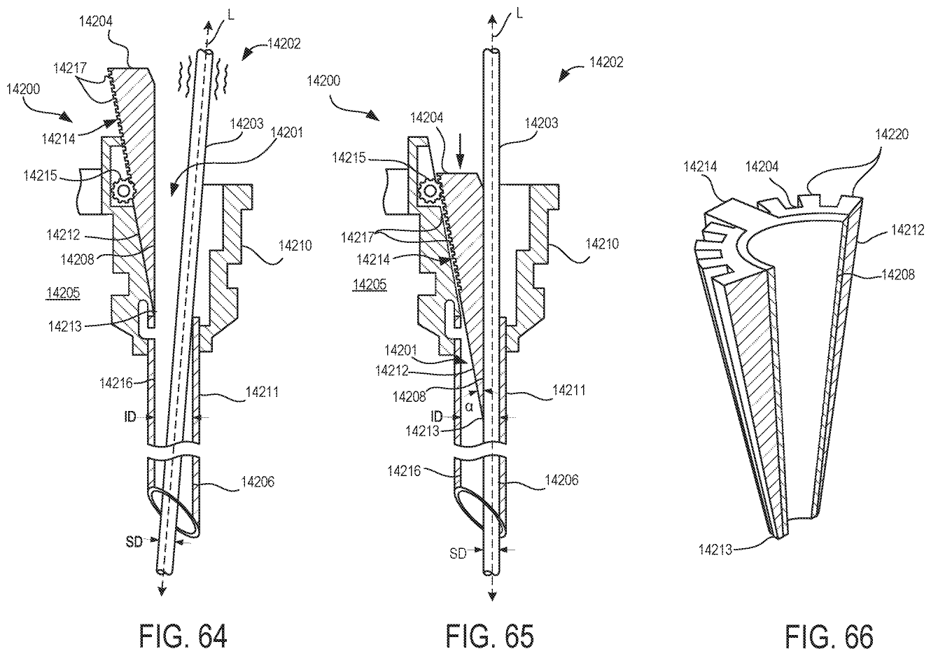

Referring primarily to FIGS. 64-66, surgical access devices such as, for example, a surgical access device 14200 are configured to facilitate insertion of various surgical instruments into a body cavity 14205 of a patient. The surgical access device 14200 includes a housing 14210 and a tubular member 14211 extending distally from the housing 14210. The tubular member 14211 and the housing 14210 define a common passageway 14201. As illustrated in FIG. 64, a shaft 14203 of a surgical instrument 14202 can be inserted through the passageway 14201 to permit an end effector of the surgical instrument 14202 to perform a surgical function in the body cavity 14205.

In many instances, as illustrated in FIG. 64, the shaft 14203 of a surgical instrument 14202 inserted through the surgical access device 14200 has a diameter “SD” that is significantly smaller than an inner diameter “ID” of the inner wall 14212 of the tubular member 14211. The size discrepancy may cause the shaft 14203 to rattle, wobble, or unintentionally change position relative to the surgical access device 14200. This wobbling effect is augmented when the surgical instrument is controlled by a robotic arm that transmits vibrations to the surgical instrument during operation. In situations where the surgical instrument 14202 and/or the surgical access device 14200 are controlled by a robotic arm, these unintended movements may prevent the robotic arm from accurately calculating a present and/or desired position of surgical instrument 14202 and/or the surgical access device 14200.

The surgical access device 14200 includes a translatable member 14204 configured to stabilize a smaller size shaft such as, for example, the shaft 14203 to prevent unintentional movements of the shaft 142023 and/or dampens any vibrations transmitted to the shaft 14203. The translatable member 14204 is movable relative to the housing 14210 between a first position (FIG. 64), which can be a proximal or starting position, and a second position (FIG. 65), which can be a distal or end position, to stabilize the shaft 14203. In the example of FIG. 65, the translatable member 14204 is configured to abut and align the shaft 14203 against an inner wall 14206 of the tubular member 14211 in the second position. As illustrated in FIGS. 64 and 65, the translatable member 14204 is configured to move the shaft 14203 into parallel alignment with the inner wall 14206 such that a longitudinal axis “L” of the shaft 14203 extends in parallel with the inner wall 14206.

In various examples, the translatable member 14204 is integral with the housing 14210. In other examples, the translatable member 14204 can be releasably coupled to the housing 14210. Any suitable fastening mechanism can be employed to releasably and repeatedly couple the translatable member 14204 to the housing 14210.

In various examples, the translatable member 14204 has a partial conical shape, as illustrated in FIG. 66. The translatable member 14204 is configured to be wedged between the shaft 14203 and an inner wall 14216 opposite the inner wall 14206 causing a first wall 14208 of the translatable member 14204 to snuggly abut against the shaft 14203, which causes the shaft 14203 to abut against and be aligned with the inner wall 14206 of the tubular member 14211, as illustrated in FIG. 65. The translatable member 14204 includes a second wall 14212 extending at an acute angle α with the first wall 14208. In the second position, a distal end 14213 of the translatable member 14204 is positioned closer to the inner wall 14206, further away from the inner wall 14216, and deeper into the passageway 14201 than in the first position. The second wall 14212 includes a translation member 14214 with translation features 14217. The translation member 14214 is movably engaged with a translation driver 14215.

In one example, as illustrated in FIGS. 64 and 65, the translatable member 14214 defines a linear gear on the second wall 14212, and the translation driver 14215 defines a rotary driver in movable engagement with the linear gear of the translation member 14214. In such example, rotational motion of the translation driver 14215 causes the translatable member 14204 to move between a number of second or end positions including the second position of FIG. 65. Accordingly, the translatable member 14204 is movably adjustable between a number of second or end positions to accommodate different size shafts of different surgical instruments. The reader will appreciate that other suitable mechanisms for transferring rotary motion to linear motion can be employed to translate the translatable member 14204 between the first position and the second position such as, for example, a slider crank mechanism or a slider crank mechanism with variable sliding length. In other examples, various suitable electro-mechanical mechanisms can be employed to translate the translatable member 14204 between the first position and the second position.

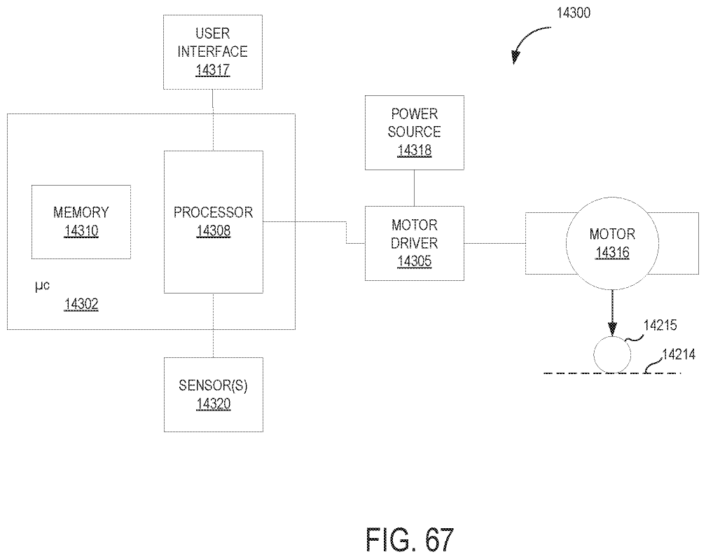

Referring to FIG. 67, a control circuit 14300 includes the controller 14302 that may generally comprise a processor 14308 (“microprocessor”) and a storage medium, which may include one or more memory units 14310, operationally coupled to the processor 14308. By executing instruction code stored in the memory 14310, the processor 14308 may control movement of the translatable member 14204 via a motor 14316, for example, in response to an input, which can be received from a user interface 14317 or one or more sensors 14320. In at least one example, the user interface 14317 is integrated with the remote command console 13370 (FIG. 6).

The sensors 14320 can be disposed onto the first wall 14208, and can be configured to detect insertion of a shaft 14203 through the passageway 14201. Further, in various examples, the sensors 14320 can be any suitable motion sensors or any other sensors capable of detecting the insertion of a shaft 14203 through the passageway 14201. Alternatively, the controller 14302 may receive input from the detection of a robotic surgical system to move to the translatable member 14204 between the first position and the second position, based on a determined position of the shaft 14203.

In various examples, the sensors 14320 include pressure sensors configured to assess the pressure exerted by the translatable member 14204 on the shaft 14203. The controller 14302 may adjust the position of the translatable member 14204 to adjust the pressure value within, or in accordance with, a predetermined threshold range.

The controller 14302 may be implemented using integrated and/or discrete hardware elements, software elements, and/or a combination of both. Examples of integrated hardware elements may include processors, microprocessors, controllers, integrated circuits, application specific integrated circuits (ASIC), programmable logic devices (PLD), digital signal processors (DSP), field programmable gate arrays (FPGA), logic gates, registers, semiconductor devices, chips, microchips, chip sets, controllers, system-on-chip (SoC), and/or system-in-package (SIP). Examples of discrete hardware elements may include circuits and/or circuit elements such as logic gates, field effect transistors, bipolar transistors, resistors, capacitors, inductors, and/or relays. In certain instances, the controller 14302 may include a hybrid circuit comprising discrete and integrated circuit elements or components on one or more substrates, for example. In certain instances, the controller 14302 may be a single core or multicore controller LM4F230H5QR.

In various forms, the motor 14316 may be a DC brushed driving motor having a maximum rotation of, approximately, 25,000 RPM, for example. In other arrangements, the motor 14316 may include a brushless motor, a cordless motor, a synchronous motor, a stepper motor, or any other suitable electric motor. A power source 14318 may supply power to the motor 14316, for example.

A motor driver 14305 in operable communication with the controller 14302 can be configured to control a direction of rotation of the motor 14316. In certain instances, the motor driver 14305 may be configured to determine the voltage polarity applied to the motor 14316 by the power source 14318 and, in turn, determine the direction of rotation of the motor 14316 based on input from the controller 14302. For example, the motor 14316 may reverse the direction of its rotation from a clockwise direction to a counterclockwise direction when the voltage polarity applied to the motor 14316 by the power source 14318 is reversed by the motor driver 14305 based on input from the controller 14302. In addition, the motor 14316 is operably coupled to the translation driver 14215 which can be rotated by the motor 14316 to move the translation member 14214 distally, toward the second position, or proximally, toward the first position, depending on the direction in which the motor 14316 rotates, for example.

In various aspects, referring primarily to FIG. 66, the translatable member 14204 includes flexible or resilient features 14220 disposed onto the second wall 14212. The features 14220 are configured to seal the access port defined through the housing 14210, as illustrated in FIG. 65, to maintain insufflation fluid within a suitable range.

As described above, robotic arms produce vibrations that can be transferred to surgical instruments controlled by the robotic arms. Such vibrations may have negative implications on the accuracy of the surgical instruments during a surgical procedure. Further, surgical instruments with shafts comprising significantly smaller diameters than receiving surgical access devices may rattle, wobble, or unintentionally change position relative to the receiving surgical access devices, which can be augmented when the surgical instruments are controlled by robotic arms that transmit vibrations to the surgical instruments during operation. To minimize the effect of vibrations of a robotic arm 14400 on a surgical instrument 14405 being controlled by the robotic arm 14400, and/or reduce wobbling or rattling, during operation, a vibration dampening mechanism 14401 is disclosed. The vibration dampening mechanism 14401 automatically adjusted a mounting assembly 14402 of the robotic arm 14400 to maintain a direct contact between a surgical access device 14403 releasably coupled to the mounting assembly 14402 and the surgical instrument 14405.

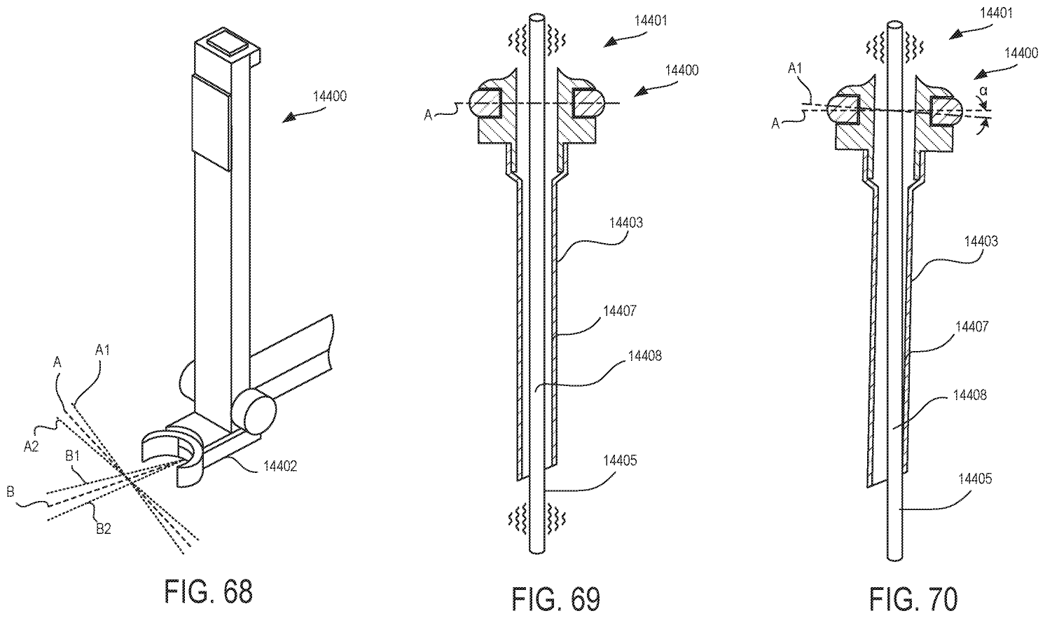

Referring primarily to FIGS. 68-70, the robotic arm 14400 is similar in many respects to other robotic arms described herein such as, for example, the robotic arms 13002, 13003 (FIG. 4), 13200 (FIG. 23). Further, the surgical access device 14403 is similar in many respects to other surgical access devices described herein such as, for example, the trocar 13250. The mounting assembly 14402 includes clamp arms 14 configured to hold the surgical access device 14403. Further, the mounting assembly 14402 is configured to slightly adjust the orientation of the surgical access device 14403 to maintain a direct contact between an inner wall 14407 of the surgical access device 14403 and a shaft of 14408 of the surgical instrument 14405 extending through the surgical access device 14403, as illustrated in FIG. 70. The direct contact allows the surgical access device 14403 to act as a vibrations dampener for the surgical instrument 14405.

In the example of FIGS. 69 and 70, the robotic arm 14400 causes the mounting assembly 14402 to be rotated with the surgical access device 14403 an angle α in a clockwise direction to establish and maintain the direct contact between the shaft 14408 and the inner wall 14407. The axis A represents the surgical access device 14403 at a neutral position. The Axis A1 represents the surgical access device 14403 in a first tilted position.

Referring to FIG. 68, the robotic arm 14400 is configured to rotate the tool mount assembly 14402 clockwise and counterclockwise to new positions defined by the axes A1 and A2 from a neutral position defined by the Axis A, for example. The robotic arm 14400 is configured to rotate the tool mount assembly 14402 up and down to new positions defined by the axes B1 and B2 from a neutral position defined by the axis B, for example. Like the robotic arms 13002, 13003 (FIG. 4) the robotic arm 14400 may be driven by electric drives that are connected to the control device 13004 (FIG. 4) for rotation of the tool mount assembly 14402 to establish and maintain a direct contact between the shaft 14408 and the inner wall 14407 of the surgical access device 14403.

In various aspects, the inner wall 14407 can include one or more pressure sensor to detect pressure applied by the inner wall 14407 onto the shaft 14408. The control device 13004 can be configured to receive input indicative of the pressure, and to adjust the position of the surgical access device 14403 in accordance with a predetermined threshold range. In various aspects, achieving or exceeding a predetermined minimum pressure threshold is indicative of the establishment of the direct contact between the shaft 14408 and the inner wall 14407.

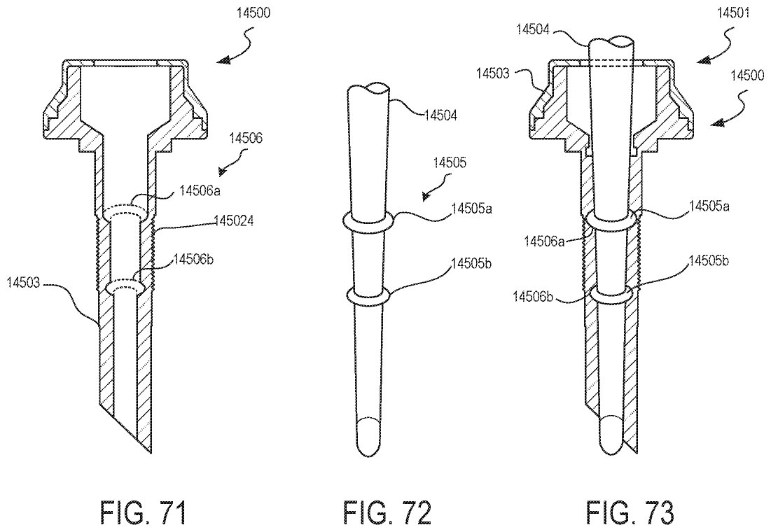

Referring to FIGS. 71-73, another vibration dampening mechanism 14501 is disclosed. Unlike the vibration dampening mechanism 14401, the vibration dampening mechanism 14501 does not require manipulating a tool mounting assembly to maintain a direct contact between a surgical instrument and a surgical access device. Instead, the dampening mechanism 14501 equips a surgical instrument such as, for example, an obturator 14504 with dampening features 14505, and a surgical access device 14500 with corresponding stabilizing compartments 14606. In various aspects, a tubular member 14503 of the surgical access device 14500 includes an outer wall that defines stability threads 14524, as illustrated in FIG. 71.

As illustrated in FIG. 73, the dampening features 14505 are received in their respective stabilizing compartments 14506. Vibrations from a robotic arm that are transferred to the obturator 14504 are absorbed and/or transferred by the dampening features 14505 to the surgical access device 14500. Further, the dampening features 14505 cooperate with the stabilizing compartments 14506 to maintain the obturator 14504 along a central axis of the surgical access device 14500. In various aspects, the dampening features 14505 include a proximal dampening feature 14505a and a distal dampening feature 14505b that are spaced apart from one another along a length of the obturator 14504. Further, the stabilizing compartments 14506 include a proximal stabilizing compartment 14506a configured to receive the proximal dampening feature 14505a and a distal stabilizing compartment 14506b configured to receive the distal dampening feature 14505b, as illustrated in FIG. 73.

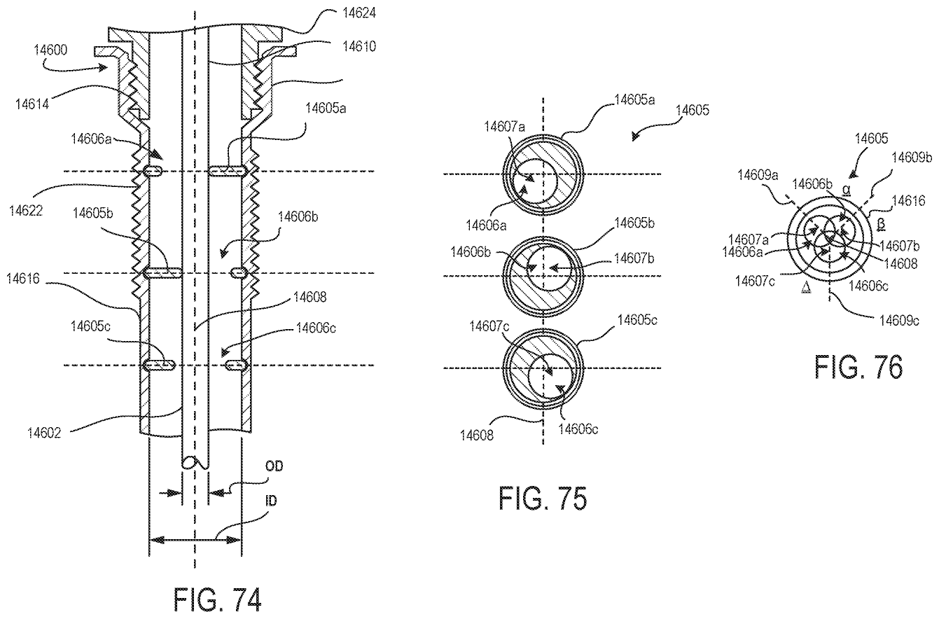

Referring to FIGS. 74-76, a surgical access device 14600 includes non-concentric instrument support features 14605 arranged along a length of the surgical access device 14600. A shaft 14602 of a surgical instrument 14610 extends through the surgical access device 14600. The shaft 14602 has an outer diameter “OD” smaller than an inner diameter “ID” of an inner wall 14620 of the surgical access device 14600. The non-concentric instrument support features 14605 cooperate to bias the shaft 14602 toward and/or maintain the shaft 14602 at a central axis 14608 defined through a common passageway 14612 of the surgical access device 14600.

As illustrated in FIG. 74, the surgical access device 14600 includes a housing 14614 and a tubular member 14616 extending distally from the housing 14614. The common passageway 14612 is defined through the tubular member 14616 and the housing 14614.

In the example illustrated in FIG. 75, the non-concentric instrument support features 14605 include a first instrument support feature 14605a that has a first opening 14606a therethrough, a second instrument support feature 14605b that has a second opening 14606b therethrough, and a third instrument support feature 14606a that has a third opening 14606c therethrough. The first opening 14606a, the second opening 14606b, and the third opening 14606c are all offset with respect to the central axis 14608 in different directions. In other words, each of the non-concentric instrument support features includes a thicker section and a thinner section around its opening.

In various aspects, the non-concentric instrument support features 14605a are made from deformable, flexible, and/or biasing materials. The thick sections are elastically deformed by the shaft 14602 and, as such, exert biasing forces against the shaft 14602 to bias the shaft 14602 toward and/or maintain the shaft 14602 at the central axis 14608. In various aspects, the non-concentric instrument support features 14605 are made, or at least partially made, from any suitable polymeric material. In various aspects, the non-concentric instrument support features 14605 comprise the same or different material compositions.

In various examples, as illustrated in FIG. 76, the first opening 14606a includes a first center 14607a that is offset from the central axis 14608 in a first direction 14609a, and the second opening 14606b includes a second center 14607b that is offset from the central axis 14608 in a second direction 14609b, and the third opening 14606c includes a third center 14607c that is offset from the central axis 14608 in a third direction 14609c. The first direction 14609a, the second direction 14609b, and the third direction 14609c extend away from the central axis 1608. In at least one example, the first direction 14609a, the second direction 14609b, and the third direction 14609c are transverse, or at least substantially transverse, to the central axis 14608. In at least one example, the first direction 14609a, the second direction 14609b, and the third direction 14609c are spaced apart by angles α, β, Δ that can be 120 degrees, as illustrated in FIG. 76.

In various aspects, one or more of the non-concentric instrument support features 14605 could form part of a seal assembly of the surgical access device 14600 causing a high insertion and extraction load but tightly holding onto the shaft 14602. In various aspects, the tubular member 14616 includes an outer wall that defines stability threads 14622, as illustrated in FIG. 74. Further, a mounting assembly 14624 of a robotic arm can be threadably engaged to the surgical access device 14600.

In various aspects, one or more instrument support features for stabilizing a surgical instrument shaft within a surgical access device are in the form of inflatable members that can be expanded to at least partially fill an empty space between the outer diameter of the shaft and the inner diameter of the surgical access device to stabilize the surgical instrument. Alternatively, the instrument support features may include inflator baffles to be charged once the surgical instrument is inserted through a surgical access device.

In various aspects, an insufflation port can be interconnected with the inflatable members. Insufflation ports typically inject a fluid such as, for example, carbon dioxide into a body cavity such as, for example, the abdominal cavity to inflate the body cavity creating space for a surgical procedure to be performed in the body cavity. In certain examples, insufflation ports can be integrated with the surgical access devices. In various aspects, an insufflation port can automatically inflate the instrument support features of a surgical access device. Fluid from the insufflation port can be transmitted to the inflatable members of a surgical access device to stabilize a surgical instrument extending through the surgical access device. In various aspects, a control circuit can be configured to detect the insertion of a surgical instrument through the surgical access device, and automatically inflate the inflatable members. Further, the control circuit can be configured to detect the removal of the surgical instrument from the surgical access device, and automatically deflate the inflatable members. The control circuit can be coupled to fluid pump, which can be activated to inflate and/or deflate the inflatable members. In certain aspects, can be configured to trigger opening and closing one or more fluid valves to inflate and/or deflate the inflatable members.

Detecting the insertion and/or removal of the surgical instrument can be accomplished by one or more suitable sensors that can be positioned along a length of the surgical access device. The sensors could be light sensors, motion sensors, pressure sensors, or any other suitable sensors. The sensors may transmit sensor signals to the control circuit indicative of the detection of the insertion and/or removal of the surgical instrument. The control circuit main then inflate or deflate the instrument support features based on the sensor signals.

In certain aspects, pressure sensors can be employed to monitor pressure exerted onto the instrument support features by a shaft of the surgical instrument. For example, pressure sensors can be positioned inside the instrument support features to detect a change in fluid pressure caused by a change in instrument side loads exerted against the instrument support features. In response, the control circuit may adjust fluid pressure within the instrument support features to improve surgical instrument stability. In other examples, pressure inside the instrument support features can be calculated based on the amount of fluid delivered to the instrument support features.

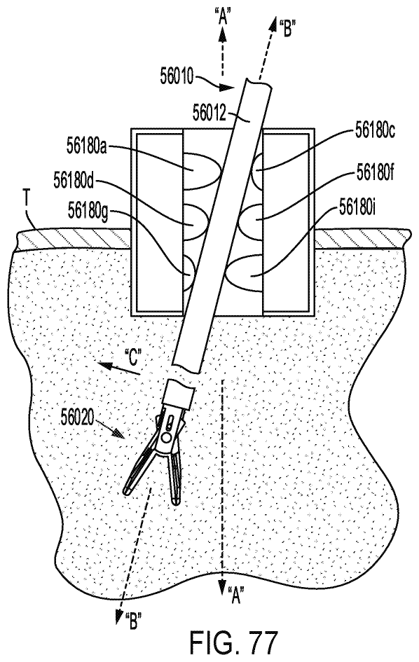

In the embodiment illustrated in FIG. 77, port assembly includes nine inflatable members 56180a-56180i associated therewith (Inflatable members 56180b, 56180e and 56180h are not shown in FIG. 77 due to the particular cross-sectional view illustrated). The inflatable members 56180a-56180i of the illustrated embodiment include a first, proximal row of three inflatable members 56180a-56180c radially disposed about interior surface of the body, a second, middle row of three inflatable members 56180d-56180f radially disposed about interior surface of the body, and a third, distal row of three inflatable members 56180g-56180i radially disposed about interior surface of the body.

A sensor is configured to communicate the orientation and positioning information of the end effector assembly 56020 with control mechanism including a controller. Moreover, the sensor is configured to communicate the difference between the current orientation and positioning of the end effector assembly 56020 with the stored (e.g., initial) orientation and positioning information. The control mechanism is configured to distribute an inflatable medium to the appropriate inflatable member(s) 56180 in order to move the shaft 56012 of the surgical device 56010 to re-orient the end effector assembly 56020, such that the end effector assembly 56020 moves to its stored (e.g., initial) orientation and position. For example, and with particular reference to FIG. 77, to tilt the end effector 56020 with respect to the longitudinal axis “A” in the general direction of arrow “C,” inflatable members 56180a and 56180i could be inflated and/or inflatable members 56180c and 56180g could be deflated. Reference may be made to U.S. patent application Ser. No. 15/520,966, now U.S. Pat. No. 10,251,672, the entire contents of which are incorporated herein by reference, for additional detailed discussion.

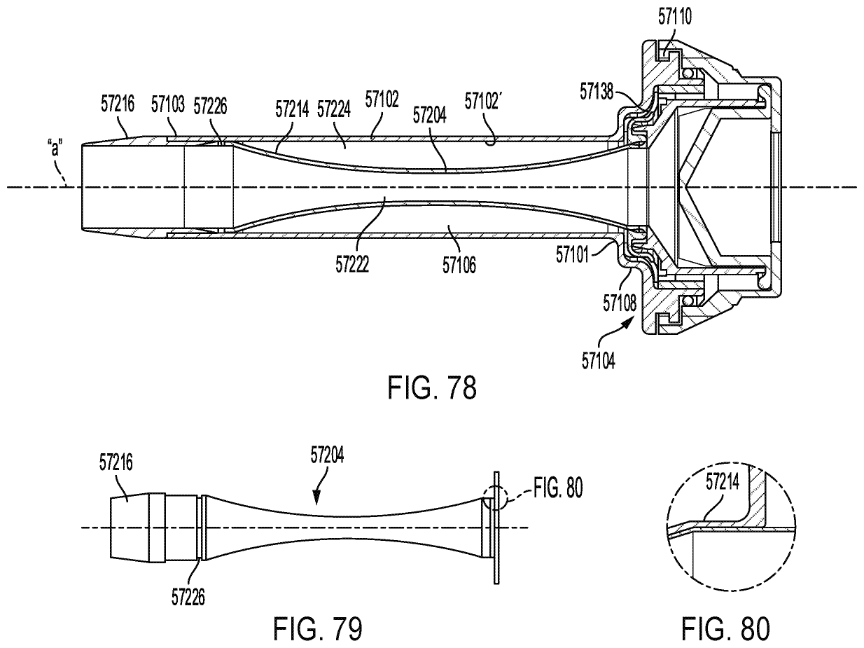

In one embodiment, referring now to FIGS. 78-80, an access apparatus, i.e., cannula assembly IOU, includes cannula sleeve 57102 having proximal and distal ends 57101, 57103 and cannula housing 57104 mounted to the proximal end 57101 of the sleeve 57102. Cannula sleeve 57102 defines a longitudinal axis “a” extending along the length of sleeve 57102. Sleeve 57102 includes an inner wall 57102′ that further defines an internal longitudinal passage 57106 dimensioned to permit passage of a surgical object such as surgical instrumentation. Sleeve 57102 incorporates sleeve flange 57108 monolithically-formed at the proximal end 57101. Sleeve 57102 may be fabricated of stainless steel or another suitable rigid material such as a polymeric material or the like. Sleeve 57102 may be clear or opaque. The diameter of sleeve 57102 may vary, but, typically ranges from 5 to 15 mm. Sleeve flange 57108 has a seal support integrally formed with or attached to the sleeve flange 57108. Sleeve flange 57108 further includes at least one circumferential recess or slot 57110 within its outer surface. Circumferential slot 57110 mates or cooperates with corresponding structure of cannula housing 57104 to secure cannula sleeve 57102 and cannula housing 57104.

Elongated seal 57204 is coaxially arranged within cannula sleeve 57102 to define an outer passageway 57224 between the elongated seal 57204 and the internal surface of cannula sleeve 57102. The outer passageway 57224 communicates with channel 57138 and port. Elongated seal 57204 further defines a gap 57226 or portion adjacent cannula tip 57216 devoid of the elastomer. The gap 57226 permits the passage of insufflation gases between outer passageway 57224 and internal passageway 57222 of elongated seal 57204. Insufflation gases are introduced from port, through channel 57138 through outer passageway 57224, out gap 57226 into the body cavity, to expand the body cavity. Alternatively or additionally, gap 57226 permits the insufflation gases to pass from outer passageway 57224 to internal passageway 57222, as well as from internal passageway 57222 into outer passageway 57224, to substantially equalize the pressure within the two locations to allow the seal to adjust to instruments of different sizes. The gap 57226 may be provided during the molding process or, alternatively, may be the result of a removal step where the elastomer is removed subsequent to molding to define the gap 57226. The gap 57226 may be created by perforating or forming a slit in the outer elastomeric material 57214. It is further envisioned that cannula sleeve 57102 may include an opening in its outer wall in communication with the outer passageway 57224 to permit passage of gases to the abdominal cavity. Reference may be made to U.S. patent application Ser. No. 12/780,494, now U.S. Pat. No. 8,070,731, the entire contents of which are incorporated herein by reference, for additional detailed discussion.

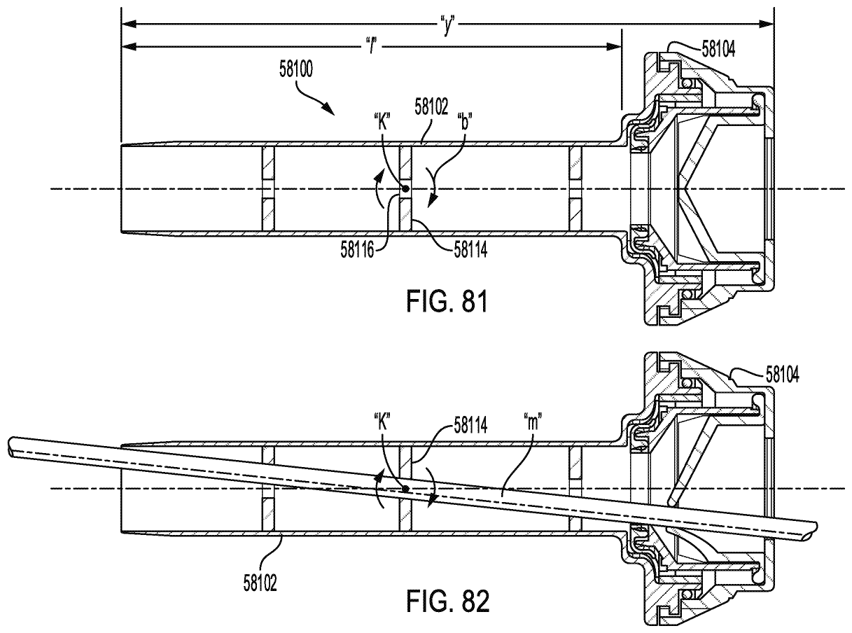

Referring now to FIGS. 81 and 82, an instrument seal 58114 will be discussed. Instrument seal 58114 is mounted within sleeve 58102 and may be a generally annular or disk-shaped element having inner seal portions defining an internal passage 58116 for reception and passage of a surgical instrument in substantial sealed relation. Internal passage 58116 may be an aperture, slit or the like adapted to permit a surgical instrument to pass through instrument seal 58114. Instrument seal 58114 may be mounted within sleeve 58102 by any conventional means envisioned by one skilled in the art including, e.g., with the use of adhesives, cements or mechanical mounting means. Instrument seal 58114 may comprise any suitable elastomeric material. In one embodiment, instrument seal 58114 comprises an elastomeric material, a fabric material, and/or combinations of these materials. The fabric material may comprise braided, woven, knitted, non-woven materials. In yet a further alternative, instrument seal 58114 is a fabric seal and is arranged so as to have a constricted area. The fabric is constructed of a material that forms a constriction or closure. The seal may also be molded with a resilient material so as to have a constriction. Instrument seal 58114 they comprise a gel or foam material. Other arrangements for instrument seal 58114 are also envisioned.

Instrument seal 58114 is disposed at the rotational center “k” of the cannula assembly 58100. The rotational center “k” may be at the axial midpoint (the midpoint of the axial length “I”) of cannula sleeve 58102, or, at the axial midpoint of the combined length “y” of the cannula sleeve 58102 and cannula housing 58104. The disposition of instrument seal 58114 at the rotational center “k” of cannula sleeve 58102 or the combined cannula sleeve 58102 and cannula housing 58104 will enable an inserted surgical instrument “in” to be manipulated through a range of motions as depicted by the directional arrows “b” in FIG. 82 (including angular movement and/or rotational movement) while minimizing distortion of the instrument seal 58114. Specifically, the surgical instrument “m” will angulate about the rotational center “k” thereby minimizing the distortion of at least the inner surface portions of instrument seal 58114 which is positioned adjacent to or exactly at the location of the rotational center “k”. This will thereby preserve the integrity of the seal formed by instrument seal 58114 about the surgical instrument “m” and substantially minimize the passage of insufflation gases through the instrument seal 58114. In addition, the disposition of instrument seal 58114 within cannula sleeve 58102 may eliminate the need for cannula housing 58104 or, in the alternative, substantially reduce the height requirement of the cannula housing 58104 in that the instrument seal 58114 does not need to be incorporated within the cannula housing 58104. Reference may be made to U.S. patent application Ser. No. 13/445,023, now U.S. Patent Application Publication No. 2012/0238827, the entire contents of which are incorporated herein by reference, for additional detailed discussion.

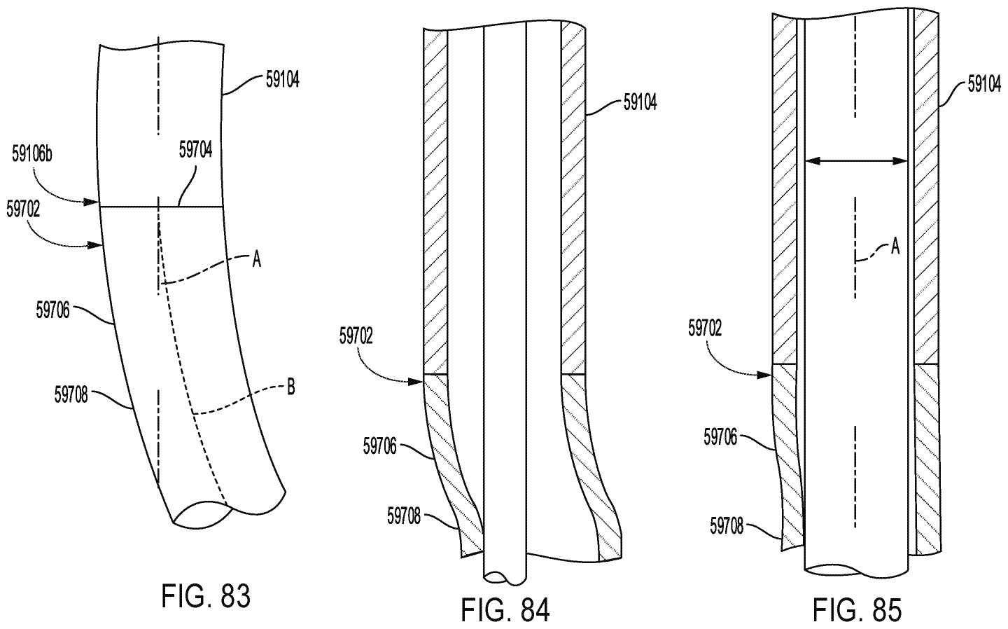

FIG. 83 is a side view of an example radial biasing device 59702 that may be used with a trocar assembly, according to one or more embodiments of the present disclosure. The radial biasing device 59702 may be coupled to or otherwise arranged at or near the distal end 59106b of the cannula 59104 at an interface 59704 between an annular body and the cannula 59104. The radial biasing device 59702 may include an annular body 59706 that also constitutes a compliant stabilizing member 59708. In the illustrated embodiment, the annular body 59706 and compliant stabilizing member 59708 are in the form of a tube or hose that extends distally from the distal end 59106b of the cannula 59104. Moreover, the annular body 59706 and compliant stabilizing member 59708 may be bent or curved such that a centerline B of the radial biasing device 59702 diverges from the centerline A of the cannula 59104 as the annular body 59706 extends distally from the distal end 59106b of the cannula 59104.

FIGS. 84 and 85 are cross-sectional side views of the radial biasing device 59702 depicting example operation, according to one or more embodiments. All or a portion of the radial biasing device 59702 may be made of a pliable or elastic material to enable the radial biasing device 59702 to transition between a generally relaxed position, as shown in FIG. 84, and an extended position, as shown in FIG. 85. Reference may be made to U.S. patent application Ser. No. 15/720,612, now U.S. Patent Application Publication No. 2019/0099201, the entire contents of which are incorporated herein by reference, for additional detailed discussion.

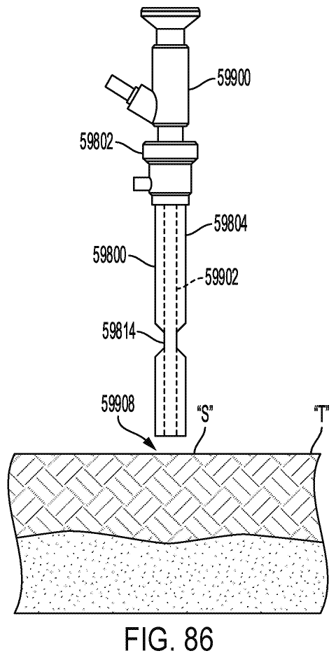

Referring now to FIG. 86, the use and function of a system will be discussed. The peritoneal cavity is first insufflated with a suitable biocompatible gas such as, e.g., CO2 gas, such that the cavity wall is raised and lifted away from the internal organs and tissue housed therein, providing greater access thereto, as is known in the art. The insufflation may be performed with an insufflation needle or similar device. Following insufflation, obturator assembly 59900 is positioned within cannula assembly 59800, specifically, first through a seal assembly (not shown), if any, and then through cannula housing 59802 and cannula member 59804, respectively. Thereafter, obturator 59902 is advanced such that contact is made between penetrating end 59908 of obturator 59902 and skin site “S” of tissue “T”. A force is then applied to the proximal end of obturator assembly 59900 such that penetrating end 59908 may puncture tissue “T”. Following penetration, obturator assembly 59900 is removed from cannula assembly 59800. Thereafter, a variety of surgical instrumentation may be inserted through cannula member 59804 of cannula assembly 59800 to carry out the remainder of the surgical procedure. Upon insertion, a substantially fluid-tight seal will be created between restrictor hinge 59814 and the surface of the instrument. Additionally, restrictor hinge 59814 may maintain the desired orientation of the instrument and may align its axis with that of cannula member 59804. Reference may be made to U.S. patent application Ser. No. 12/468,271, now U.S. Pat. No. 8,197,446, the entire contents of which are incorporated herein by reference, for additional detailed discussion.

While several forms have been illustrated and described, it is not the intention of the applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor comprising one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A surgical visualization system, comprising:
an imaging device comprising an imaging lens;
a lens cleaning system;
a user interface; and
a control circuit configured to:
monitor a parameter indicative of lens transparency of the imaging lens; and
present, through the user interface, a lens transparency level based on the parameter.

2. The surgical visualization system of claim 1, wherein the parameter is a temperature of a patient cavity.

3. The surgical visualization system of claim 1, wherein the parameter is light refraction of an infrared light refracted transverse to the imaging lens.

4. The surgical visualization system of claim 1, wherein the imaging device comprises a capacitive sensor, and wherein the parameter relates to measurements of the capacitive sensor.

5. The surgical visualization system of claim 1, wherein the parameter is an image integrity level of a frame captured by the imaging device through the imaging lens.

6. The surgical visualization system of claim 1, wherein the parameter is an image blurriness level of a frame captured by the imaging device through the imaging lens.

7. The surgical visualization system of claim 1, wherein the control circuit is further configured to detect an excessive deterioration of the lens transparency level based on the parameter.

8. The surgical visualization system of claim 7, wherein the control circuit is further configured to automatically activate the lens cleaning system to remedy the excessive deterioration of the lens transparency level.

9. The surgical visualization system of claim 7, wherein detecting the excessive deterioration of the lens transparency level is achieved by comparing values of the parameter with a predetermined threshold.

10. A surgical visualization system, comprising:
an imaging device comprising an imaging lens;
a lens cleaning system;
a user interface; and
a control circuit configured to:
monitor a parameter indicative of lens transparency of the imaging lens;
detect an excessive deterioration of the lens transparency based on the parameter; and
automatically activate the lens cleaning system to remedy the excessive deterioration of the lens transparency.

11. The surgical visualization system of claim 10, wherein the parameter is a temperature of a patient cavity.

12. The surgical visualization system of claim 10, wherein the parameter is light refraction of an infrared light refracted transverse to the imaging lens.

13. The surgical visualization system of claim 10, wherein the imaging device comprises a capacitive sensor, and wherein the parameter relates to measurements of the capacitive sensor.

14. The surgical visualization system of claim 10, wherein the parameter is an image integrity level of a frame captured by the imaging device through the imaging lens.

15. The surgical visualization system of claim 10, wherein the parameter is an image blurriness level of a frame captured by the imaging device through the imaging lens.

16. A surgical visualization system, comprising:
an imaging device comprising an imaging lens;
a lens cleaning system;
a user interface; and
a control circuit configured to:
monitor a parameter indicative of lens occlusion of the imaging lens; and
provide, through the user interface, a lens occlusion level based on the parameter.

17. The surgical visualization system of claim 16, wherein the parameter is a temperature of a patient cavity.

18. The surgical visualization system of claim 16, wherein the parameter is light refraction of an infrared light refracted transverse to the imaging lens.

19. The surgical visualization system of claim 16, wherein the parameter is an image blurriness level of a frame captured by the imaging device through the imaging lens.

20. The surgical visualization system of claim 16, wherein the control circuit is further configured to detect an excessive occlusion of the imaging lens based on the parameter.
